(19) United States
(12) Patent Application Publication
(10) Pub. No.: US 2025/0255807 A1
(43) Pub. Date: Aug. 14, 2025

(54) COMPOSITIONS AND METHODS FOR DELIVERING THERAPEUTICS TO THE HEART

(71) Applicant: NORTH CAROLINA STATE UNIVERSITY, Raleigh, NC (US)

(72) Inventors: Ke Cheng, Raleigh, NC (US); Dashuai Zhu, Raleigh, NC (US); Zhenhua Li, Raleigh, NC (US)

(21) Appl. No.: 18/262,861

(22) PCT Filed: Jan. 25, 2022
(86) PCT No.: PCT/US2022/013670

§ 371 Date: Jul. 25, 2023

Related U.S. Application Data
(60) Provisional application No. 63/141,134, filed on Jan. 25, 2021.

(51) Int. Cl. A61K 9/06; A61K 35/28; A61K 35/545; A61K 38/18; A61K 47/36; A61K 47/64; A61M 5/42; C12N 15/113

(52) U.S. Cl. CPC A61K 9/06; A61K 35/28; A61K 35/545; A61K 38/1825; A61K 47/36; A61K 47/645; A61M 5/427; C12N 15/113; C12N 2310/141

(57) ABSTRACT

The present disclosure provides compositions and methods related to the delivery of therapeutic medicines to the heart for treating a cardiac injury, such as those that occur due to a myocardial infarction (MI). In particular, the present disclosure provides novel hydrogel-based compositions that safely and effectively deliver a therapeutic agent to the pericardial cavity of the heart to treat the cardiac injury.

### CROSS REFERENCE TO RELATED APPLICATIONS

This application claims priority to and the benefit of U.S. Provisional Patent Application Ser. No. 63/141,134 filed Jan. 25, 2021, which is incorporated herein by reference in its entirety and for all purposes.

### FIELD

The present disclosure provides compositions and methods related to the delivery of therapeutic medicines to the heart for treating a cardiac injury, such as those that occur due to a myocardial infarction (MI). In particular, the present disclosure provides novel hydrogel-based compositions that safely and effectively deliver a therapeutic agent to the pericardial cavity of the heart to treat the cardiac injury.

### BACKGROUND

Cardiovascular diseases remain the number one cause of death in western societies. When experiencing a major heart attack, or myocardial infarction (MI), a patient can lose about one billion healthy cardiomyocytes. The ischemic area will be infiltrated by inflammatory cells and later on replaced by cardiac fibrosis. Once advanced heart failure occurs, a heart transplantation is the only option. Regenerative therapies using live cells, proteins, and nuclei acids, aims at altering the trajectory of adverse heart remodeling and promoting de novo cardiac repair. However, it is difficult to deliver therapeutics to the heart, with high efficiency and low invasiveness and cost. A cardiac patch can effectively deliver therapeutics to the heart yet such procedures normally require open chest surgery.

The heart is an organ deeply embedded in the thoracic cavity. There are several ways to delivery therapeutics to the heart. For example, intravenous (IV) injection is quite safe and convenient (no anesthesia is needed), yet has a poor cardiac retention of the therapeutics. In contrast, intramyocardial (IM) injection (direct injection of therapeutics into the myocardium) can deliver a sizable amount of drug into the heart, but usually requires open chest surgery or sophisticated systems such as NOGA mapping coupled with transendocardial injection. Intracoronary (IC) injection can be readily performed by an interventional cardiologist with local anesthesia. However, cardiac retention is not ideal and is only slightly better than IV injection. Recently, tissue engineering approaches have shed some light on improving biodistribution in the heart. Laying a cardiac patch on the surface of the heart can normally generate the greatest cardiac retention. However, such procedures are difficult to perform, quite invasive, and not suitable for patients with mild-to-moderate heart diseases. In addition, the therapeutics in the patch can leak into the thorax cavity and/or cause adhesion to the thorax wall. Therefore, there is a need to develop improved materials and methods that can deliver a therapeutic composition to the heart in a minimally invasive fashion and with optimal biodistribution in the heart.

### SUMMARY

Embodiments of the present disclosure include a method for treating or preventing a cardiac injury in a subject. In accordance with these embodiments, the method includes delivering a hydrogel-based composition into a portion of a pericardial cavity of a subject, wherein the composition comprises at least one therapeutic agent; and improving at least one aspect of myocardial cells or tissue in the subject.

In some embodiments, the method is preformed using an imaging device, and wherein the composition is biocompatible. In some embodiments, the composition is biocompatible.

In some embodiments, the composition is delivered via intrapericardial (iPC) injection. In some embodiments, the method is performed before or after a separate medical procedure.

In some embodiments, the method is performed after the subject has suffered a myocardial infarction. In some embodiments, the method is performed to prevent cardiac injury associated with ischemic reperfusion.

In some embodiments, the composition forms a patch-like structure within the pericardial cavity.

In some embodiments, delivery of the composition to the pericardial cavity of the subject causes the hydrogel-based composition to degrade and release the at least one therapeutic agent.

In some embodiments, the at least one therapeutic agent comprises a growth factor, a microRNA, a microRNA mimic, an exosome, a cell, and any combinations or derivatives thereof. In some embodiments, the growth factor is Fibroblast Growth Factor (FGF); in some embodiments, the microRNA mimic is miR-21, miR-125, miR-146, or any combination thereof; in some embodiments, the exosome is a mesenchymal stem cell (MSC)-derived exosome; in some embodiments, the cell is an induced pluripotent stem cell-derived cardiac progenitor cell (iPS-CPCs); and in some embodiments, wherein the cell is a mesenchymal stem cell (MSC).

In some embodiments, the hydrogel-based composition is at least one of a hyaluronic acid (HA)-based hydrogel, a decellularized extracellular matrix (ECM) hydrogel, a polyvinyl alcohol (PVA)-based hydrogel, and any combinations or derivatives thereof.

In some embodiments, the at least one aspect of myocardial cells or tissue that is improved comprises increased myocardiocyte survival, decreased myocardiocyte apoptosis, increased myocardiocyte proliferation, increased myocardial differentiation, increased angiogenesis, reduced ischemia, improved myocardiocyte function, and any combinations thereof.

In some embodiments, the subject is a human.

Embodiments of the present disclosure also include a hydrogel-based composition comprising for treating a cardiac injury. In accordance with these embodiments, the composition includes a hydrogel component; and at least one therapeutic agent.

In some embodiments, the hydrogel component comprises at least one of a hyaluronic acid (HA)-based hydrogel component, a decellularized extracellular matrix (ECM) hydrogel component, a polyvinyl alcohol (PVA)-based hydrogel component, and any combinations or derivatives thereof.

In some embodiments, the at least one therapeutic agent comprises a growth factor, a microRNA, a microRNA mimic, an exosome, a stem cell, and any combinations or derivatives thereof.

In some embodiments, the at least one therapeutic agent comprises Fibroblast Growth Factor (FGF), and wherein the hydrogel component comprises a polyvinyl alcohol (PVA)-based hydrogel component.

In some embodiments, the hydrogel-based composition further comprises N¹-(4-boronobenzyl)-N³-(4-boronophenyl)-N¹,N¹,N³,N³-tetramethylpropane-1,3-diaminium (TSPBA), and wherein exposure of the composition to reactive oxygen species (ROS) cleaves the TSPBA from the PVA-based hydrogel component and releases the at least one therapeutic agent.

In some embodiments, the concentration of PVA ranges from about 7% to about 11% of the composition, and wherein the concentration of TSPBA ranges from about 1% to about 5% of the composition.

In some embodiments, the at least one therapeutic agent comprises miR-21, miR-125, miR-146, or any combination thereof, and wherein the hydrogel component comprises a decellularized extracellular matrix (ECM) hydrogel component.

In some embodiments, the miR-21, miR-125, miR-146, or any combination thereof, is present in the composition at a concentration ranging from about 2 nM to about 2 μM.

In some embodiments, the miR-21, miR-125, miR-146, or any combination thereof, is chemically modified with an HIV TAT peptide.

In some embodiments, the ECM hydrogel component is present in the composition at a concentration ranging from about 5 mg/ml to about 25 mg/ml.

In some embodiments, the at least one therapeutic agent comprises a mesenchymal stem cell (MSC)-derived exosome, and wherein the hydrogel component comprises a hyaluronic acid (HA)-based hydrogel component.

In some embodiments, the HA-based hydrogel component comprises methacrylic anhydride (MA) cross-linked to HA.

In some embodiments, the at least one therapeutic agent comprises a mesenchymal stem cell (MSC), and wherein the hydrogel component comprises a decellularized extracellular matrix (ECM) hydrogel component.

In some embodiments, the at least one therapeutic agent comprises an induced pluripotent stem cell-derived cardiac progenitor cell (iPS-CPCs), and wherein the hydrogel component comprises a decellularized extracellular matrix (ECM) hydrogel component.

Embodiments of the present disclosure also include use of a hydrogel-based composition comprising at least one therapeutic agent for the treatment and/or prevention of a cardiac injury. Embodiments of the present disclosure also include use of a hydrogel-based composition comprising at least one therapeutic agent for the manufacture of a medicament to treat and/or prevent a cardiac injury.

In accordance with these embodiments, the hydrogel component comprises at least one of a hyaluronic acid (HA)-based hydrogel component, a decellularized extracellular matrix (ECM) hydrogel component, a polyvinyl alcohol (PVA)-based hydrogel component, and any combinations or derivatives thereof. In some embodiments, the at least one therapeutic agent comprises a growth factor, a microRNA, a microRNA mimic, an exosome, a stem cell, and any combinations or derivatives thereof. In some embodiments, the at least one therapeutic agent comprises Fibroblast Growth Factor (FGF), and wherein the hydrogel component comprises a polyvinyl alcohol (PVA)-based hydrogel component. In some embodiments, the hydrogel-based composition further comprises N¹-(4-boronobenzyl)-N³-(4-boronophenyl)-N¹,N¹,N³,N³-tetramethylpropane-1,3-diaminium (TSPBA), and wherein exposure of the composition to reactive oxygen species (ROS) cleaves the TSPBA from the PVA-based hydrogel component and releases the at least one therapeutic agent. In some embodiments, the concentration of PVA ranges from about 7% to about 11% of the composition, and wherein the concentration of TSPBA ranges from about 1% to about 5% of the composition. In some embodiments, the at least one therapeutic agent comprises miR-21, miR-125, miR-146, or any combination thereof, and wherein the hydrogel component comprises a decellularized extracellular matrix (ECM) hydrogel component. In some embodiments, the miR-21, miR-125, miR-146, or any combination thereof, is present in the composition at a concentration ranging from about 2 nM to about 2 μM. In some embodiments, the miR-21, miR-125, miR-146, or any combination thereof, is chemically modified with an HIV TAT peptide. In some embodiments, the ECM hydrogel component is present in the composition at a concentration ranging from about 5 mg/ml to about 25 mg/ml. In some embodiments, the at least one therapeutic agent comprises a mesenchymal stem cell (MSC)-derived exosome, and wherein the hydrogel component comprises a hyaluronic acid (HA)-based hydrogel component. In some embodiments, the HA-based hydrogel component comprises methacrylic anhydride (MA) cross-linked to HA. In some embodiments, the at least one therapeutic agent comprises a mesenchymal stem cell (MSC), and wherein the hydrogel component comprises a decellularized extracellular matrix (ECM) hydrogel component. In some embodiments, the at least one therapeutic agent comprises an induced pluripotent stem cell-derived cardiac progenitor cell (iPS-CPCs), and wherein the hydrogel component comprises a decellularized extracellular matrix (ECM) hydrogel component.

### BRIEF DESCRIPTION OF THE DRAWINGS

FIGS. 1A-1C: iPC injection mitigates immune response as compared to IM injection in rats. (a) Schematic illustration of iPC injection and in situ formation of cardiac patch. (b) H & E staining showing the formation of a cardiac patch 7 days after iPC injection (dash lines). Intramyocardial (IM) injection of iPS-CPCs in ECM hydrogel caused massive infiltration of immune cells (arrows). iPC injection reduced immune cell infiltration and promoted regional formation of blood vessels (asterisks) in the patch. (c) Representative microscopic images showing the presence of neutrophils, CD4 and CD8 T cells and corresponding quantitation. Scale bar, 60 μm, n=4 animals per group, **p<0.01.

FIGS. 2A-2J: iPC injected iPS-CPCs contribute to cardiac regeneration and repair in a rat model of MI. (a-c) Hearts were collected 7 days after injection, and in vivo differentiation of iPS-CPCs into cardiac and vascular lineages after iPC injection were detected. (d, e) Representative confocal microscopic images showing immunostaining of α-SMA and CD31 in the hearts 7 days after injection and corresponding quantification. (f) Representative heart sections with Masson's trichrome staining (red=healthy tissue; blue=scar) 4 weeks after treatment. From the Masson's trichrome-stained images, infarct size (g) and LV wall thickness (h) were quantified. (i, j) iPC injection of iPS-CPCs improved cardiac functions. Scale bar, 60 μm, n=4 animals per group, *p<0.05, **p<0.01, ***p<0.001, ***p<0.0001.

FIGS. 3A-3F: iPC injection of MSC-exosomes in MA-HA hydrogel in a mouse model of acute MI. (a) Schematic showing intrapericardial delivery of exosomes for MI therapy. (b) Synthesis of MA-HA hydrogel. (c) In vitro gelation of MA-HA hydrogel under UV irradiation and SEM images of MA-HA hydrogel before and after gelation. (d) Fluorescent imaging of mice after iPC injection of DiD-labelled exosomes with or without MA-HA hydrogel. (e) Quantitative data of fluorescence intensity and accordingly, (f) the area under curve was measured. Data are expressed as mean±SD, n=3 animals per group, ***p<0.001.

FIGS. 4A-4J: iPC delivery of exosomes stimulated epicardium-derived repair in a mouse model of MI. (a-e) 3 days after iPC injection, hearts were collected for histological analysis. (a) Epicardial distribution of exosomes after iPC injection. (b) Z-Stack images showing the uptake of exosomes by epicardial cells by immunostaining of podoplanin, a marker for epicardium. Scale bar, 60 μm. (c) H&E staining showing the epicardial spreading of HA hydrogel after iPC injection. Moreover, the thickness of epicardium layer was measured accordingly. Scale bar, 60 μm. (d) iPC injection of exosomes stimulated accumulation of WT-1 positive epicardium-derived progenitor cells (EPDC). (e) Epicardial cell proliferation was detected by co-localization of Ki67 (a cell proliferation marker) and podoplanin. The numbers of Ki67/podoplanin double-positive cells were counted. Scale bar, 60 μm. (f) Masson trichrome staining was performed 4 weeks after MI, and (g) the fibrotic area as well as (h) the infarct wall thickness was quantified. (i, j) Echocardiography measurement of cardiac function, including left ventricular ejection fraction (LVEF) and fractional shortening (LVFS) after various treatment. Data are expressed as mean±SD, *p<0.05, **p<0.01, ***p<0.001, ****p<0.0001, n=6 animals per group.

FIGS. 5A-5G: Minimally invasive iPC injection of therapeutics in pigs and iPC access in human patients. (a) Schematic illustration of minimally invasive delivery of therapeutics into pericardial cavity with the aid of endoscope in pigs. (b) Representative ex vivo imaging of pig hearts 3 days after intrapericardial injection of exosomes. (c) Confocal microscopy images showing uptake of exosomes by cardiomyocytes 3 days after iPC injection. Scale bar, 60 μm. (d) Analysis of blood cells, (e) inflammatory cytokines in pericardial fluid and (f) Serum chemistry on hepatic, renal, and cardiac functions. Data are expressed as mean±SD, *p<0.05, n=3 animals. (g) Minimally invasive iPC access in a human patient who underwent a standard LARIAT procedure. First, a lateral view angiogram was obtained under fluoroscope (1). After that, using a small bore (0.018″) access needle, iodinated contrast was injected to visualize the border of the pericardial space (2). A wire was advanced into the pericardial space (3). Next, a series of dilations were performed prior to the introduction of the access sheath in this case or foreseeable a catheter can be advanced here for intrapericardial injection (4).

FIGS. 6A-6B: Preparation and characterization of ECM hydrogel. (a) Preparation of ECM hydrogel (up panel left to the right, fresh heart tissues, decellularized heart tissues and lyophilized decellularized heart tissues), and H-E staining for the confirmation of successful decellularization (bottom panel). (b) In vitro gelling of ECM solution at 37° C. (left panel) and representative SEM images showing the distinct structure of ECM hydrogel (right panel, up: before gelling, bottom: after gelling).

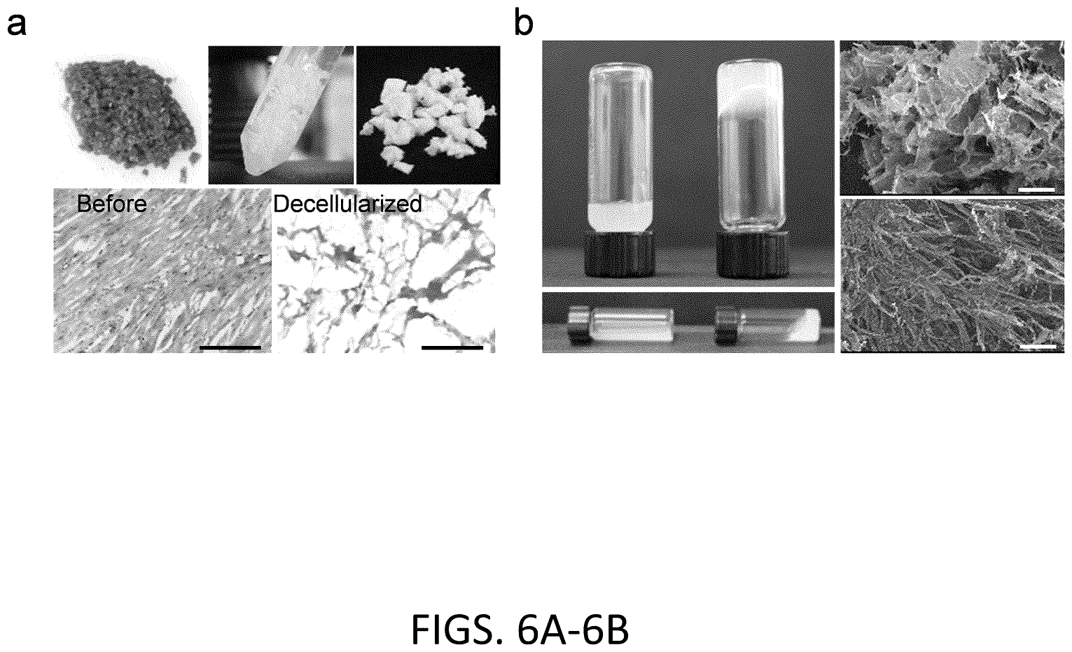

FIG. 7: Biocompatibility of ECM hydrogel after injection into the pericardial cavity. ECM hydrogel was directly injected into pericardial cavity. 3 days later, H&E staining was performed for the evaluation of inflammatory infiltration. Scale bar, 100 μm.

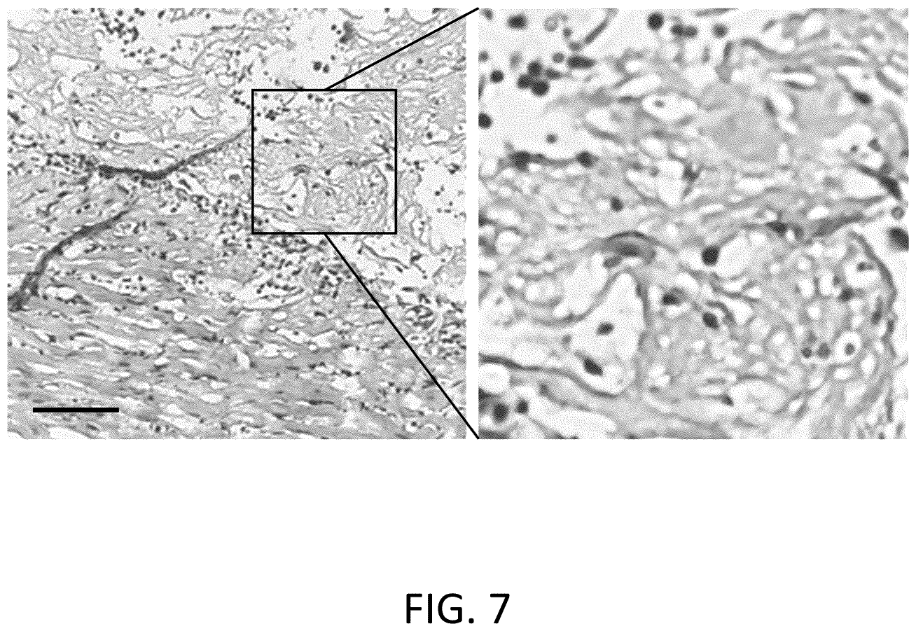

FIGS. 8A-8C: In vitro proliferation and differentiation of iPS-CPCs. (a) In vitro proliferation of iPS-CPCs when incubated with bFGF. (b and c) In vitro differentiation detection of iPS-CPCs into cardiomyocytes.

FIG. 9: Representative Masson's trichrome-stained heart sections 4 weeks after treatment. Scale bar, 100 μm.

FIG. 10: Echocardiography determination of cardiac functions. Representative M-mode images of left ventricle. Baseline cardiac functions were measured 2 hours after surgery.

FIGS. 11A-11B: Characterization of MA-HA and exosomes. The MA modification of HA hydrogel was confirmed by mass spectrum (a). Exosome morphology was confirmed with TEM (b). Scale bar, 1 μm.

FIG. 12: iPC injection of exosomes promotes EPDC differentiation. Expressions of stem/progenitor and stromal cell markers in epicardial cells after intrapericardial exosome injection. Scale bar, 60 μm.

FIGS. 13A-13C: iPC-injected exosomes are observed in the lymph nodes. After iPC injection of DiD-labeled exosomes, an accumulation of exosomes was found in the cardiac-draining mediastinal lymph nodes (MLN) and inguinal lymph nodes (ILN). Delivery of exosome in HA hydrogel reduced exosome loss to the lymph nodes. Data are expressed as mean±SD, n=5 animals per group.

FIG. 14: iPC injection of exosomes reduces apoptosis. Four weeks after therapy, TUNEL staining was performed to detect apoptotic cells and TUNEL positive cell numbers were counted. Scale bar, 100 μm. Data are expressed as mean±SD, *p<0.05, ****p<0.0001, n=6 animals per group.

FIGS. 15A-15B: iPC injection of exosomes mitigates cardiac remodeling after MI. 3 months after iPC injection of exosomes in HA hydrogel, Masson's trichrome staining (a) revealed larger wall thickness and less scar in the treated animals and echocardiography (b) indicated augmented cardiac functions. All Data are expressed as mean±SD, *p<0.05, ****p<0.0001, n=3 animals per group.

FIG. 16: Minimally invasive iPC injection in pigs. Stilled images taken during the iPC injection procedures in pigs.

FIG. 17: Cytokines array analysis of inflammation in pericardial fluid after iPC injection in pigs. Pericardial fluid was harvested before and after iPC injection for inflammatory cytokine analysis.

FIG. 18: iPC injection in ECM hydrogel enhances the cardiac retention of mesenchymal stem cells (MSCs).

FIGS. 19A-19F: Injecting GFP-MSCs into the pericardial cavity of infarcted mouse hearts shows feasibility and safety. A. Schematic image showing the difference of injection sites between two delivery routes. B. H&E images of the injected cells combined with ECM gel (white arrows). scale bar, 100 μm. C. Representative SEM image of the ECM gel and fluorescence images of GFP-MSCs in culture. Left panel, high power field of the gel, scale bar, 20 μm. Right panel, low power field of the gel, scale bar, 100 μm. D. Representative IVIS fluorescence images of GFP-MSCs in the ECM gel compared to the empty gel. E. Representative stereomicroscopy images showing no pericardial effusion after the IPC injection. F. Summary of overall physical condition and survival rates of the IPC group and IM group.

FIGS. 20A-20D: IPC delivery of MSCs improves cardiac function. A. Schematic image showing the study design. Echocardiography was measured at 2 days, 14 days and 42 days after the surgery. B. Representative M-mode echocardiography images at 2 days, 14 days and 42 days after the MI from one animal in each group. C. LVEF was measured at 2 days, 14 days and 42 days after the MI. n=6 in each group. D. LVFS was measured at 2 days, 14 days and 42 days after the MI. n=6 in each group. All data are means±SD. Comparisons among groups were performed using two-way ANOVA followed by post hoc Bonferroni test. The comparisons between samples are indicated by lines, and the statistical significance is indicated by asterisks above the lines. *P<0.05 and **P<0.01.

FIGS. 21A-21L: IPC delivery of MSCs yielded 10-fold better retention than IM delivery. A. Representative fluorescent images showing the beginning of GFP-MSCs (green) migration into the myocardium (red) at 2 days after the IPC injection. Scale bar, 100 μm. B. Representative fluorescence images showing the process of GFP-MSCs (green) migration into the myocardium (red) from 2 days to 2 weeks after the IPC injection and IM injection. Scale bar, 100 μm. C. Schematic image showing migration process of MSCs in the myocardium. D. Standard curve representing the relationship between cell numbers and GFP concentration in the GFP-MSCs in vitro (from ELISA). E. Quantification of retention rate based on the standard curve and ELISA from IPC group and IM group. F. Comparisons between retention rates reported in the literature at different time ranges (0-2 h, 3-4 h, 18-24 h) and retention rates from the IPC injections at 1 week. G. Quantification of average distances of MSCs migration based on the IHC images from IPC group and IM group. H. Quantification of maximum distances of MSCs migration based on the IHC images from IPC group and IM group. I. Quantification of migration percentages based on the increase of distances during time range of 0-2 days, 2-7 days and 7-14 days. (increased distance: total distance) J. IVIS bioluminescence images of Luc-MSCs placed on Petri-dishes in vitro at increasing cell numbers and Luc-MSCs distributed in vivo at baseline and 1 weeks after the IPC and IM injections compared to empty gel injection as a control. K. Standard curve representing the relationship between cell numbers and bioluminescence in the Luc-MSCs in vitro. L. Quantification of retained cell number based on bioluminescence at baseline and 1 week after IPC injections. All data are means±SD. Comparisons among groups were performed using one-way ANOVA followed by post hoc Bonferroni test. The comparisons between samples are indicated by lines, and the statistical significance is indicated by asterisks above the lines. *P<0.05 and **P<0.01.

FIGS. 22A-22F: IPC delivery of MSCs leads to better myocardial regeneration. A. Representative fluorescence images of cell apoptosis detected by terminal deoxynucleotidyl transferase-mediated deoxyuridine triphosphate nick end labeling (TUNEL) expression (red). Scale bars, 50 μm. B. Quantitation of TUNEL+ cells in A. (n=3). C. Representative fluorescence images showing Ki67+ expression (red) in the myocardium. Scale bars, 50 μm. C. Standard curve representing the relationship between cell numbers and GFP concentration in the GFP-MSCs in vitro (from ELISA). D. Quantitation of Ki67+ cells in C. (n=3). E. Representative fluorescence images of vascular regeneration indicated by α-SMA expression (green). Scale bars, 100 μm. F. Quantitation of α-SMA+ blood vessels in E. (n=3). All data are means±SD. Comparisons among groups were performed using one-way ANOVA followed by post hoc Bonferroni test. The comparisons between samples are indicated by lines, and the statistical significance is indicated by asterisks above the lines. *P<0.05 and **P<0.01.

FIGS. 23A-23F: Establishment of CD63-RFP exosome labeling system in vitro. A. Schematic image showing the genetic modification of MSCs based on a specific vector. B. Representative fluorescence images showing the RFP-exosomes (red) uptake in cardiomyocytes (white) in co-culture with ER-MSCs (green) in vitro. scale bar, 100 μm. C. Representative flow cytometry plots of ER-MSCs for RFP, CD63, CD44, CD90 markers. D. Western blot analysis of ER-MSCs and control (unmodified MSCs) for RFP marker. E. Quantification of RFP expression based on western blot analysis in D. for ER-MSCs and control. F. Representative fluorescence images showing co-localization of RFP-exosomes (red) and exosome-specific markers (green), including CD63, CD81, TSG101, and Alix. scale bar, 50 μm. All data are means±SD. Comparisons among groups were performed using one-way ANOVA followed by post hoc Bonferroni test. The comparisons between samples are indicated by lines, and the statistical significance is indicated by asterisks above the lines. *P<0.05 and **P<0.01.

FIGS. 24A-24G: Paracrine activity by IPC-delivered MSCs. A. Representative fluorescence images showing the RFP-exosomes (red) uptake in cardiomyocytes (green) in mouse MI hearts injected with ER-MSCs by the IPC route or IM route. scale bar, 100 μm. B. Quantitation of RFP+ cells based on A. for the IPC group and IM group. C. Representative fluorescence images showing co-localization of RFP-exosomes (red) and exosome-specific markers TSG101 (green) D. Quantitation of RFP expression based on ELISA. E. Western blot analysis of cardiac tissue from IPC injection or IM injection of ER-MSCs for RFP and CD63 markers. F. Quantification of CD63 expression based on western blot analysis in E. for IPC group and IM group. G. Quantification of RFP expression based on western blot analysis in E. for IPC group and IM group. All data are means±SD. Comparisons among groups were performed using one-way ANOVA followed by post hoc Bonferroni test. The comparisons between samples are indicated by lines, and the statistical significance is indicated by asterisks above the lines. *P<0.05 and **P<0.01.

FIGS. 25A-25C: Concept of Gel-bFGF and screening for the optimal FGF for heart repair. (A) Schematic illustration of Gel-bFGF fabrication and overall strategy (Copyright WILEY-VCH Verlag GmbH & Co. KGaA, 69469 Weinheim, Germany, 2018). (B) Confocal microscopy images showing FGFs promote NRCM proliferation. 0 FBS and 10% FBS were included as negative and positive controls, respectively. Scar bar, 50 μm. (C) Quantification of Ki67-positive NRCMs incubated with various FGFs (n=5). **** indicates p<0.0001.

FIGS. 26A-26E: ROS-responsive gel preparation and its effects on cardiomyocytes. (A) Gel formed with various concentrations of PVA and ROS-sensitive linker (TSPBA); (B) Photographs of liquid gel formed from 9% of PVA and 3% of TSPBA; (C) bFGF release behavior over time at various ROS concentrations; (D) Confocal fluorescence microscopy images showing co-incubation of bFGF-loaded Gel and NRCMs with or without H₂O₂. Pink nuclei: merge of red (Ki67) and blue (4,6-diamidino-2-phenylindole dihydrochloride, DAPI). Scar bar, 50 μm; (E) Quantification of Ki67-positive NRCMs (n=5). **** indicates p<0.0001.

FIGS. 27A-27E: Intrapericardial delivery of Gel-bFGF and cardiac retention. (A) Timeline of animal studies; (B) Images taken during the injection; (C) Ex vivo IVIS imaging of hearts after intrapericardial delivery of bFGF alone or Gel-bFGF at baseline, 2 d and 4 d, n=3; (D) Quantification of fluorescence intensities of bFGF in the hearts; (E) Confocal fluorescence microscopy images showing released bFGF into the myocardium. Scar bar, 50 μm. White arrows indicate the approximate of released bFGF to cardiomyocytes. **** indicates p<0.0001.

FIGS. 28A-28F: Gel-bFGF injection promotes angiomyogenesis. Confocal microscopy images of (A) Ki67, (B) Von Willebrand factor (vWF), and (C) CD31 staining of heart sections 4 weeks after injection. N indicates normal area and I indicates infarct area. Scale bars, 50 μm. (D-F) Quantitation of Ki67, vWF, and CD31-positive cells (n=4). *** and **** indicates p<0.005 and p<0.0001, respectively.

FIGS. 29A-29F: Functional benefits of Gel-bFGF therapy in I/R rats. (A) Representative Masson's trichrome-stained myocardial sections 4 weeks after treatment; (B) Quantitative analyses of viable myocardium from the Masson's trichrome images; (C) Left ventricular internal diameter at end-diastole (LVIDd) and (D) end-systole (LVIDs) measured by echocardiography at 4 weeks; (E) Left ventricular ejection fractions (LVEFs) and (F) fractional shortening (LVFS) of rat after 4 week-treatment (n=5). ** and **** indicates p<0.01 and p<0.0001, respectively.

FIGS. 30A-30J: Pilot safety study of Gel-bFGF in pigs and feasibility of iPC access in a human patient. (A) A schematic showing the pig study design. (B) Photograph showing the port sites for injection and camera. (C) Photographs showing the process of iPC injection in pigs. (D) Schematic showing the preparation of myocardium slices for ex vivo fluorescent imaging. (E) Representative ex vivo fluorescent imaging of pig hearts 3 days after intrapericardial injection of Gel-bFGF@AF594 (n=3). (F) Confocal fluorescence microscopy images showing bFGF@AF594 in myocardium. (G) Cytokine array analysis of the inflammatory cytokine concentrations in the pericardial fluid 3 days after treatment. (H) Quantitative results from G. (I) A schematic showing minimally invasive iPC access in human patients. (J) Fluoroscopy images from a patient who underwent a LARIAT procedure. First, a lateral view angiogram is obtained which reveals the location of the apex of the right ventricle (1). Next, using a small bore (0.018″) access needle, iodinated contrast is used to mark the border of the pericardial space (2). After entering the space with a needle, a wire was advanced into the pericardial space and a serial dilations were performed prior to the introduction of the access sheath that can be used for intrapericardial injection (3).

FIG. 31: Schematic diagram of the molecular structures of three types of FGFs.

FIG. 32: Representative chromatograms of four types of FGFs.

FIG. 33: Representative mass spec data of four types of FGFs.

FIG. 34: Effects of various concentrations of four FGFs on cardiomyocyte proliferation. N=3, **** indicates p<0.0001.

FIG. 35: ¹H-NMR of the ROS-responsive linker (TSPBA).

FIG. 36: SEM images of PVA-TSPBA Gel and the final product bFGF-loaded Gel.

FIGS. 37A-37B: ROS-triggered gel disassembly. PVA-TSPBA gel was incubated with ROS at different concentrations. Pictures were taken with daylight (A) or UV-light (B).

FIG. 38: Amplitude sweep of elastic modulus (G′) at 1 Hz and 25° C.

FIG. 39: Frequency and flow sweep of storage (G′) and loss (G″) moduli of samples.

FIG. 40: Frequency Sweep for gels with different ratios of PVA and TSPBA-Phase Angle (δ) at 25° C.

FIG. 41: Oscillatory temperature ramp measurements of bFGF-loaded gel made from 9% PVA and 3% TSPBA.

FIG. 42: Effects of gel on NRCM viability.

FIG. 43: Ex vivo imaging of Gel-bFGF distribution in multiple organs. Ex vivo IVIS images of major organs including heart (H), liver (Li), spleen (S), lung (Lu), and thymus (T) after intrapericardial delivery of bFGF alone or Gel-bFGF at 0, 2 d and 4 d.

FIGS. 44A-44B: Measurement of H₂O₂ concentration in normal hearts or I/R hearts at different time points. (A) H₂O₂ concentrations measured from infarct heart tissue; (B) H₂O₂ concentration measured from pericardial fluid.

FIGS. 45A-45B: Gel biodistribution and anti-apoptotic effects. (A) H&E stain of heart tissue after intrapericardial administration of Gel-bFGF; (B) TUNEL staining to study the cardioprotective effects of Gel-bFGF. N indicates normal area and I indicates infarct area. Scar bar, 50 μm; (C) Quantification of the apoptosis cardiomyocytes.

FIGS. 46A-46B: iPC injection of Gel-bFGF promotes angiogenesis. Confocal microscopy images showing Von Willebrand factor (vWF) (A) and CD31 staining (B) of the hearts 4 weeks after therapy. N indicates normal area and I indicates infarct area. Scale bars, 50 μm. (The images in this figure show larger fields of view than the images in FIG. 4.)

FIG. 47: Macrophage infiltration study. Representative fluorescent images showing the presence of infiltrated CD68-positive macrophage (red) 28 days after various treatments. Scale bar, 50 μm.

FIGS. 48A-48D: Baseline values of cardiac functions and chamber dimensions. (A) Left ventricular internal diameter at end-diastole (LVIDd) and (B) end-systole (LVIDs) measured by echocardiography at baseline (4 h post-I/R); (C) Left ventricular ejection fractions (LVEFs) and (D) fractional shortening (LVFS) of rat at baseline (4 h post-I/R).

FIGS. 49A-49B: Toxicity of Gel-bFGF injection in pigs. (A) Blood chemistry and (B) hematology in the serum of pigs before and after Gel-bFGF treatment.

### DETAILED DESCRIPTION

Embodiments of the present disclosure include compositions and methods related to the delivery of therapeutic medicines to the heart for treating a cardiac injury, such as those that occur due to a myocardial infarction (MI). In particular, the present disclosure provides novel hydrogel-based compositions that safely and effectively deliver a therapeutic agent to the pericardial cavity of the heart to treat the cardiac injury.

A cardiac patch can be an effective way to delivery therapeutics to the heart. However, such procedures are normally invasive and difficult to perform. As described herein, a method to utilize the pericardial cavity as a natural “mold” for in situ cardiac patch formation after intrapericardial (iPC) injection of therapeutics in biocompatible hydrogels was developed and tested. In some embodiments, using rodent models of myocardial infarction (MI), results provided herein have demonstrated that iPC injection is an effective and safe method to deliver hydrogels containing induced pluripotent stem cells-derived cardiac progenitor cells (iPS-CPCs) or mesenchymal stem cells (MSCs)-derived exosomes. After injection, the hydrogels formed cardiac patch-like structure in the pericardial cavity, mitigating immune response and increasing the cardiac retention of the therapeutics. With robust cardiovascular regeneration and stimulation of epicardium-derived repair, the therapies mitigated cardiac remodeling and improved cardiac functions post MI. Furthermore, results provided herein have demonstrated the feasibility of minimally-invasive iPC injection in a clinically-relevant porcine model as well as in human patients. These results establish iPC injection as a safe and effective method to deliver therapeutics to the heart for cardiac repair.

Although cell therapy has shown potential efficacy in the treatment of heart diseases, one challenges is low cellular retention rate and poor engraftment in the site of administration. A myriad of studies confirm that cell retention plays a crucial role in the success of cell-mediated cardiac repair and regeneration. As described further herein, experiments were conducted to perform a head-to-head comparison on cell retention and therapeutic benefits of intramyocardial (IM) injection and intrapericardial (IPC) injection of adult stem cells. Mouse green fluorescent protein (GFP)-labeled mesenchymal stem cells (MSCs) were combined in extracellular matrix (ECM) hydrogel and injected into the pericardial cavity or the myocardium of the heart of C57BL/6 mice that had been subjected to a myocardial infarction. Echocardiographs were performed at 2 days, 2 weeks, and 6 weeks after the cell injections, to monitor the cardiac function of both short-term and long-term recovery. Mouse hearts were harvested at 2 days, 1 week, and 2 weeks after cell injections for histological evaluation. An ELISA assay was used to assess cellular retention ex vivo, cooperated by IVIS live imaging in vivo. Additionally, CD63-RFP exosome labeling system was established through lentiviral transduction and confirmed in vitro. ERL-MSCs were injected into the mouse MI hearts via IPC route in comparison with IM, to evaluate the paracrine activity of MSCs injected. Results demonstrated that cardiac function was significantly enhanced in the short term (2 week) and the long term (6 week). Confirmed by ELISA assay and in vivo IVIS imaging, the retention of MSCs injected via IPC route (42.5±7.4%) was 10-fold greater than that of MSCs injected intramyocardially (4.4±1.3%). Additionally, immunohistochemistry data revealed better cellular proliferation, less apoptosis, and better vascular regeneration in the myocardium after IPC delivery of MSCs. RFP-labelled exosomes, secreted by MSCs, were absorbed by cardiomyocytes at higher rates when MSCs were injected via IPC route, compared to the results from IM injections. Therefore, IPC cell delivery route led to better cardiac repair in the mouse MI model. This result was attributed to higher cell retention and engraftment after cell transplantation. The exosome labelling system used revealed more extensive paracrine activity in MSCs after IPC injections, which could possibly explain the improvement in cardiac function.

Timely reperfusion of the ischemic myocardium is the most effective way to treat myocardial infarction. However, blood reperfusion to the ischemic tissues leads to an overproduction of toxic reactive oxygen species (ROS), which can further exacerbate myocardial damage on top of ischemic injury. ROS has been used as a diagnostic marker and therapeutic target for ischemia-reperfusion (I/R) injury and as an environmental stimulus to trigger drug release. In the present disclosure, a ROS-sensitive cross-linked poly (vinyl alcohol) (PVA) hydrogel was synthesized and used to deliver basic fibroblast growth factor (bFGF) for myocardial repair. The therapeutic gel was injected into the pericardial cavity. Upon delivery, the hydrogel spread on the surface of the heart and form an epicardial patch in situ. No suture or glue was needed since the pericardial cavity served as a natural “mold” to hold the hydrogel patch. In a rat model of I/R injury, bFGF released from the gel could penetrate the myocardium. Such intervention protected cardiac function and reduced fibrosis in the post-I/R heart, with enhanced angiomyogenesis. Furthermore, results provided herein demonstrated the safety and feasibility of minimally-invasive injection and access into the pericardial cavity in both pigs and human patients, respectively.

Taken together, embodiments of the present disclosure have demonstrated the safety, efficacy, and clinical feasibility of iPC injection of therapeutics for cardiac repair. iPC injection can be performed by an experienced cardiologist in a fairly short period of time and only conscious sedation is needed. Results provided herein have confirmed that the technique is versatile as it can be used to deliver a variety of different therapeutics using various types of biomaterials. The delivery can achieve an ideal bio-distribution in the myocardium while not causing safety concerns. Given clinical trials on cardiac regeneration are currently hindered by the lack of delivery efficiency, the results provided herein demonstrate iPC injection as a new route for therapeutic administration.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The terms “administration of” and “administering” a composition as used herein refers to providing a composition of the present disclosure to a subject in need of treatment (e.g., antiviral treatment). The compositions of the present disclosure may be administered by oral, parenteral (e.g., intramuscular, intraperitoneal, intravenous, ICV, intracisternal injection or infusion, subcutaneous injection, nebulization, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition in the patient (e.g., viral infection). The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

Formulations suitable for administration include, for example, aqueous sterile injection solutions, which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

The term “derived from” as used herein refers to cells or a biological sample (e.g., blood, tissue, bodily fluids, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease. As described further herein, “treatment” and “prevention” includes use of a hydrogel-based composition comprising a therapeutic agent for the treatment and/or prevention of a cardiac injury in a subject (e.g., a human subject), as well as use of a hydrogel-based composition comprising a therapeutic agent for the manufacture of a medicament to treat and/or prevent a cardiac injury.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, cardiovascular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. THERAPEUTIC COMPOSITIONS AND METHODS OF DELIVERY

Cardiovascular tissue engineering holds great promise for heart regeneration and repair. Among them, a cardiac patch can be an excellent carrier to deliver stem cells and other therapeutic agents to the heart. Yet, deployment of a cardiac patch typically requires open chest surgery. Minimally invasive delivery of cardiac patches has been reported before. However, these procedures require special shape memory materials. In addition, direct transplantation of a cardiac patch to the epicardium disrupted the pericardium, which plays vital roles in cardiac repair after myocardial infarction. In the present disclosure, hydrogels (e.g., thermosensitive hydrogels) containing various therapeutics were injected into the pericardial cavity. This is called intrapericardial (iPC) injection. After injection, the in situ gelling process takes process and the hydrogel forms a cardiac patch-like structure in the pericardial cavity. This is very much like the “injection-molding” process used in the plastic industry. As described further herein, iPS derived cardiac progenitor cells (iPS-CPCs), mesenchymal stem cells (MSCs), mesenchymal stem cell (MSC)-derived exosomes, microRNAs, microRNA mimics, and growth factors, are among the therapeutic agents that can be delivered using the methods disclosed herein.

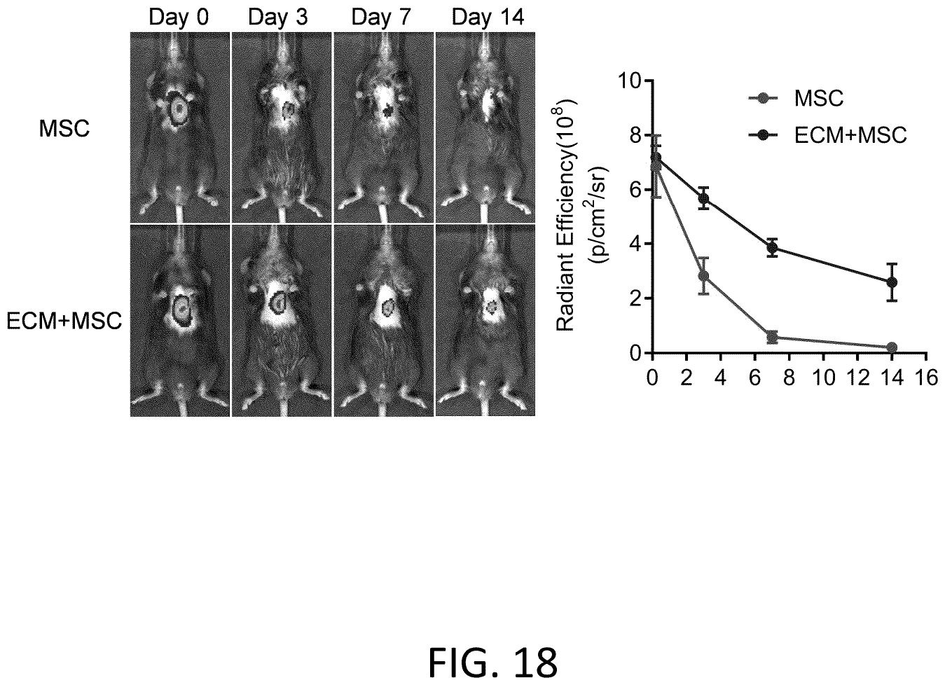

For example, iPS-CPCs are able to proliferate and have the capacity to differentiate into mature cardiomyocytes as well as the vascular lineages (endothelial cells and smooth muscle cells). In addition, paracrine activities of progenitor/stem cells are also major contributors to cardiac repair. It has also been established that MSC-derived exosomes carrying proteins, nuclear acids and other constituents are active players of paracrine activities. Post-MI inflammation and cardiac remodeling can be modulated by exosomes treatment. Exosomal transfer of miR-21, miR-125, miR-146 and other bioactive components improved cardiac repair by enhancing angiogenesis and cardiomyocyte survival. Despite the encouraging results, poor survival caused by immune rejection and low retention rate have been the critical obstacles hindering clinical translation. Therefore, using iPC injections, higher cardiac retention of mesenchymal stem cells (see, e.g., FIG. 18) and MSC exosomes was achieved, and there was less immune rejection to iPS-CPCs.

Previous studies used non-hydrogel Gelfoam or saline to deliver adult stem cells, exosomes, or growth factors into the pericardial cavity to achieve cardiac repair. However, in the present disclosure, decellularized porcine heart ECM and MA-HA hydrogels were used as biomaterial carriers to deliver therapeutics to the pericardial cavity. Given the synergy between biomaterials and therapeutics in cardiac repair and the demonstrated feasibility of minimally invasive procedures to perform such interventions, this strategy represents an advancement to the field and offers new knowledge regarding the safety and efficacy of intrapericardial injection. In the present disclosure, decellularized porcine heart ECM and MA-HA hydrogels were used to deliver therapeutics via iPC injection, of which the ECM hydrogel is now used in clinical trials, and HA being the most abundant extracellular component in the pericardium was commonly used in biomedical studies by crosslinking with MA, an effective UV photoinitiator that proved biomedical safe.

Pericardial tamponade is one common medical emergency that caused by the builds up of fluid in pericardial cavity. Under physiological conditions, balanced generation and drainage of pericardial fluid offer the heart with lubrication and protection. However, chest trauma and open chest surgery and other procedures that breaking the balance with increased generation and decreased absorption result in the occurrence of tamponade. In the present disclosure, thermosensitive ECM hydrogel and pre-crosslinked HA hydrogel were used as the carriers for various therapeutics. In addition, minimally invasive iPC procedures preserve the intact pericardial structure. No tamponade events were recorded in any of the study subjects.

As demonstrated in the human data described further herein, iPC injection of therapeutics can be performed in a way similar to the standard LARIAT procedure. During the LARIAT procedure, a local anesthetic is used to numb the area under the breastbone. After the area is numbed, catheters are advanced into the pericardial space. Compared to the NOGA mapping guided transendocardial injection, which has been challenging and needs expensive/special instrumentation, iPC injection can be performed under fluoroscope which is universally available in most of the cardiovascular medicine units worldwide.

The question of how to best deliver cells has been at the forefront of the field of heart regeneration ever since its advent. The goal is to find the delivery strategy that maximizes therapeutic efficacy and minimizes risks, as better cell delivery methods will increase the survival rate of transplanted cells in the heart. In particular, while IC and IV injections are minimally invasive, they can lead to vascular occlusions and are notorious for poor engraftment rates. IM delivery, on the other hand, offers higher cell retention but requires a risky open chest surgery and has questionable efficacy, and this route has a relatively uncertain efficiency according to an overview of the past preclinical clinical studies.

Therefore, in the present disclosure, the IPC delivery route was explored in detail and compared to IM delivery. Using a mouse MI model, a head-to-head comparison between this injection method and the common IM injection method was performed. The IPC delivery route resulted in stronger heart repair in the infarcted mice, which was attributed its success in increasing cell retention and engraftment. After the IPC injection, the exosome labeling system used herein revealed extensive paracrine activity including the release of exosomes, which is thought to be the principle reason for the improved of cardiac function.

Myocardial infarction, among other ischemia heart diseases, is the major cause of mortality and morbidity for subjects with heart diseases. Timely reperfusion of the ischemic myocardium is the most effective way to treat myocardial infarction. However, blood reperfusion to the ischemic tissues can lead to an overproduction of toxic reactive oxygen species (ROS), which can further exacerbate myocardial damage on top of ischemic injury. ROS has been used as a diagnostic marker and therapeutic target for ischemia-reperfusion (I/R) injury and as an environmental stimulus to trigger drug release. As described further herein, a ROS-sensitive cross-linked poly (vinyl alcohol) (PVA) hydrogel was synthesized, and it was used to deliver basic fibroblast growth factor (bFGF) to a subject's heart for myocardial repair. The therapeutic gel can be injected into the pericardial cavity, and upon delivery, the hydrogel spreads on the surface of the heart and forms an epicardial patch in situ. No suture or glue is needed since the pericardial cavity serves as a natural “mold” to hold the hydrogel patch. For example, in a rat model of I/R injury, bFGF released from the gel penetrated the myocardium. Such intervention protected cardiac function and reduced fibrosis in the post-I/R heart, with enhanced angiomyogenesis. Furthermore, embodiments of the present disclosure demonstrated the safety and feasibility of minimally-invasive injection and access into the pericardial cavity in both pigs and human patients, respectively.

Generally, there are mainly four delivery routes to target heart, which include intramyocardial (i.m.) injection, intracoronary (i.c.) injection, intravenous (i.v.) injection, and epicardial cardiac patch placement, which normally requires open chest surgery. Those methods have their own advantages and disadvantages. For example, i.m. injection has been the most straightforward way to administer therapeutics directly into the heart and with good cardiac retention. However, the drawback is that it normally requires open-chest surgery, unless sophisticated endomyocardial injection coupled by NOGA mapping is used. Those systems are only available in major academic research hospitals, limiting its wide use for cardiac drug delivery. The greatest advantage of i.v. delivery route is its simplicity, feasibility, and excellent safety profile. However, it suffers from extreme low delivery efficiency and retention in the heart. Although using catheters for i.c. infusion can deliver drugs directly to the culprit coronary vessel that irrigates the infarct region, its cardiac retention performance is somewhat between i.m. and i.v. injections. The use of a cardiac patch is a tissue engineering approach involving layering a scaffolding material (containing therapeutics such as stem cells, growth factors, and exosomes) on the surface of the heart. Studies suggest this can generate the highest heart retention. However, the development of such a patch is usually quite invasive.

Pericardium is a double-walled sac that gives protection against infection and provides the lubrication for the heart. The space between the two layers (serous & fibrous pericardium) is called pericardial cavity. The pericardial cavity is filled with pericardial fluid. Direct injection into the pericardial cavity (e.g., intrapericardial injection or iPC injection) has been employed to deliver stem cells, exosomes, or growth factors in a non-hydrogel Gelfoam or saline for experimental cardiac repair.

An important consideration is that successful iPC injection requires the use of biomaterials to help secure the therapeutics in the pericardial space for sustained release as the material degrades. As described herein, poly (vinyl alcohol) (PVA) was used as the building block of a hydrogel-based biomaterial. PVA is FDA-approved for a variety of medical applications. Another important consideration in treating cardiac injury is that timely reperfusion of the ischemic myocardium is considered the most effective way to save the patient's life. However, as recognized in the art, blood reperfusion into the ischemic tissues leads to an overproduction of toxic ROS, which will further exacerbate initial tissue damage; this is generally regarded as the main culprit of ischemia-reperfusion (I/R) injury. Therefore, I/R-induced ROS has been considered as a diagnostic marker and also therapeutic target.

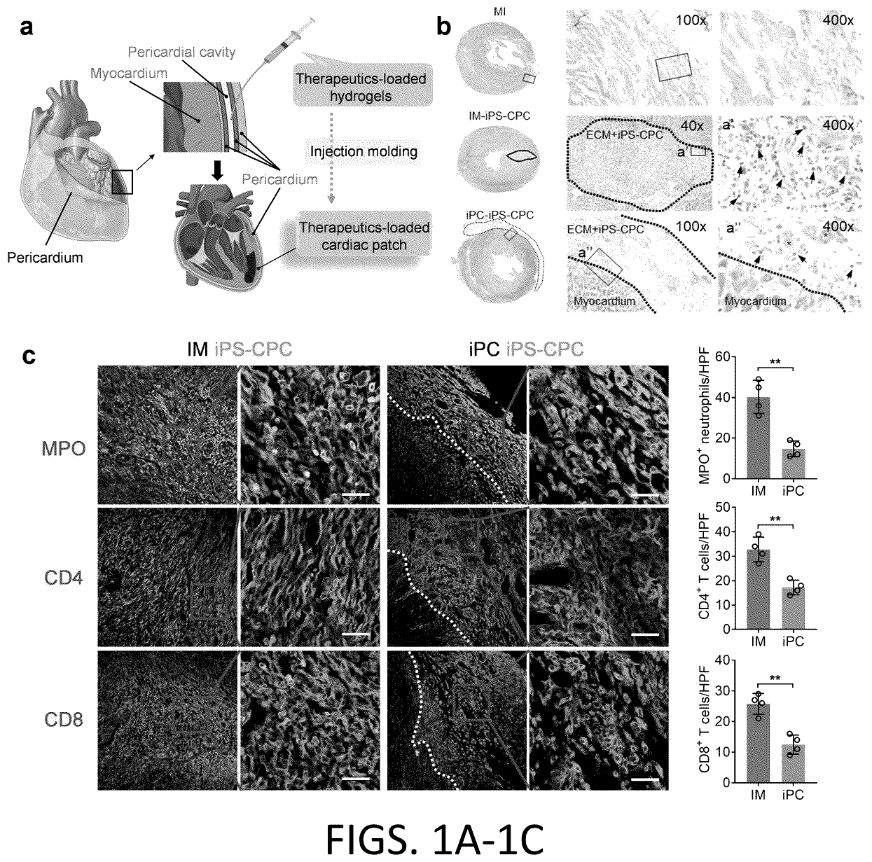

ROS-responsive drug delivery systems have been widely studied in the fields of cancer therapy, immunotherapy, and gastrointestinal (GI) diseases. However, the concentrations of ROS in the aforementioned microenvironments were low and this limits further application of such ROS-triggered drug release. In contrast, I/R heart injury leads to a rapid accumulation and sustained production of ROS which could be used as an drug release trigger. As described further herein, an bFGF-loaded, ROS-responsive hydrogel (Gel-bFGF) was developed, and it was directly injected it into the pericardial cavity as a strategy for heart repair (see, e.g., FIG. 1). The logic behind this approach is that the ROS-sensitive, cross-linked PVA-based hydrogel compositions described herein will deliver a therapeutic agent by degrading under the presence of ROS to release the bFGF into the myocardium in an “on-demand” fashion, thus providing effective dosing of a therapeutic agent for treating a cardiac injury.

Stem cells derived from multiple sources have been studied for injured heart repair. Despite the promising preclinical results, clinical efficacy of stem cell transplantation is hampered by limitations such as low retention, lack of targeting, storage instability, and low cell survival rate. Emerging evidence supports the benefits of bFGF in treating ischemic cardiovascular disease, owing to their essential roles in angiogenesis and cardiac protection. However, clinical translation of bFGF therapy for ischemia heart disease has been hindered at least in part by short half-life period and poor delivery of bFGF drugs to the heart. Neither intravenous infusion or intracoronary injection can get sufficient drug to the injured myocardium, due to strong wash-out effects, while intramyocardial normally requires open-chest surgery unless sophisticated endomyocardial injection coupled by NOGA mapping is used. iPC injection, a non-invasion delivery route, has been utilized for the delivery of therapeutic agents in clinic.

These findings showed that the loaded bFGF would be released in responsive to H₂O₂ and promote the proliferation of NRCM in vitro. In vivo, it was confirmed that the introduction of gel enhanced the retention of bFGF in pericardial cavity, which then facilitated bFGF to penetrate epicardium and bind to myocardium in both rat model and pig model. These results showed bFGF-loaded gel significantly inhibited the apoptosis of cardiomyocytes while promoted their proliferation. The enhanced myocardial angiogenesis and cardiac function were observed as well intrapericardial delivery of Gel-bFGF. However, iPC delivery required the presence of an intact pericardial space while the majority of patients with heart attract had to previously undergone coronary artery bypass surgery which typically obliterated pericardial space. Given the minimally invasive nature of iPC injection, it is anticipated that this therapy can be used acutely (e.g., during or right after PCI procedures) to repen the patient's vessel. Experiments were performed to investigate the ROS levels post-MI in rats. ROS elevation persists for a few days after the injury and was sufficient to release the bFGF into the myocardium. Furthermore, the gel would still slowly degrade even under normal ROS levels therefore eventually all FGF would be released.

The Gel-bFGF possessed the capacity to long-term release of bFGF in responsive to the over-produced ROS in pericardial cavity caused by reperfusion. Given the synergy between hydrogels and therapeutics in cardiac repair and the minimally invasive nature of the proposed procedure, this strategy represents an advancement to the field of cardiac biomaterials and drug delivery.

The present study offers translational values. As demonstrated in the pivotal human studies described further herein, iPC access of therapeutics can be performed during the standard LARIAT procedure. In such procedure, delivery catheters are advanced into the pericardial space under fluoroscope. NOGA mapping guided transendocardial injection has been challenging and needs special instrumentation. In contrast, iPC injection can be performed in all hospitals which house cardiac catherization labs.

Embodiments of the present disclosure include a method for treating and/or preventing a cardiac injury in a subject. In accordance with these embodiments, the method includes delivering a hydrogel-based composition comprising at least one therapeutic agent into a portion of a pericardial cavity of a subject. As described further herein, delivery of the composition can improve at least one aspect of myocardial cells or tissue in the subject, thereby treating and/or preventing cardiac injury in the subject.

In some embodiments, the method is preformed using an imaging device, such as an in vivo imaging device (e.g., fluoroscope), which facilitates proper delivery of the compositions of the present disclosure to the pericardial cavity. Any imaging system or instrument known in the art can be used. Since the pericardial cavity is filled with pericardial fluid, it serves as a natural “mold” for injectable hydrogels to form a uniform cardiac patch-like structure that is able to cover a portion of the heart. Intrapericardial (iPC) procedures are normally performed for epicardial catheter mapping and ablation or for other diagnosis purposes. However, as described further herein, the methods of the present disclosure include delivering therapeutics as a biocompatible hydrogel to the pericardial cavity to form a cardiac patch-like structure in situ, without the need of any suture or glue. After injection, hydrogel degradation can lead to sustained release of therapeutics into the myocardium for cardiac repair. With the aid of a fluoroscope, iPC access and injection in humans can be performed with only one incision on the chest under local anesthesia.

Therefore, in some embodiments, the compositions of the present disclosure form a patch-like structure within the pericardial cavity. In some embodiments, delivery of the composition to the pericardial cavity of the subject causes the hydrogel-based composition to degrade and release the at least one therapeutic agent. In some embodiments, the method is performed before or after a separate medical procedure. In some embodiments, the method is performed after the subject has suffered a myocardial infarction. In some embodiments, the method is performed to prevent cardiac injury associated with ischemic reperfusion.

In accordance with these embodiments, the compositions of the present disclosure are generally considered to be biocompatible. Biocompatible or biocompatibility generally refers to the ability of the compositions of the present disclosure to perform their intended function in treating and/or preventing cardiac injury, with a desired degree of incorporation in the subject, and without eliciting any significant or long-term undesirable local or systemic effects in that subject.

In some embodiments, the at least one therapeutic agent comprises a growth factor, a microRNA, a microRNA mimic, an exosome, a cell, and any combinations or derivatives thereof. In some embodiments, the growth factor is Fibroblast Growth Factor (FGF). In some embodiments, the microRNA mimic is miR-21, miR-125, miR-146, or any combination thereof. In some embodiments, the exosome is a mesenchymal stem cell (MSC)-derived exosome. In some embodiments, the cell is an induced pluripotent stem cell-derived cardiac progenitor cell (iPS-CPCs). In some embodiments, wherein the cell is a mesenchymal stem cell (MSC). As would be recognized by one of ordinary skill in the art based on the present disclosure, other therapeutic agents can also be delivered using the methods and compositions of the present disclosure, as long as the therapeutic agent(s) is generally considered to be biocompatible with the subject (e.g., a human subject).

In some embodiments, the hydrogel-based composition is at least one of a hyaluronic acid (HA)-based hydrogel, a decellularized extracellular matrix (ECM) hydrogel, a polyvinyl alcohol (PVA)-based hydrogel, and any combinations or derivatives thereof. As would be recognized by one of ordinary skill in the art based on the present disclosure, other components can be included in the compositions of the present disclosure, as long as they are generally considered to be biocompatible with the subject (e.g., a human subject) and do not interfere with the ability of the therapeutic agent(s) to treat and/or prevent cardiac injury.

As described further herein, the compositions and methods of the present disclosure can treat and/or prevent a cardiac injury when delivered to the pericardial cavity of a subject (e.g., a human subject). In some embodiments, treating and/or preventing a cardiac injury includes improving at least one aspect of myocardial cells or tissue. Improving at least one aspect of myocardial cells or tissue includes, but is not limited to, increasing myocardiocyte survival, decreasing myocardiocyte apoptosis, increasing myocardiocyte proliferation, increasing myocardial differentiation, increasing angiogenesis, reducing ischemia, improving myocardiocyte function, and any combinations thereof.

Embodiments of the present disclosure also include a hydrogel-based composition for treating and/or preventing a cardiac injury. In accordance with these embodiments, the composition includes a hydrogel component and at least one therapeutic agent. Embodiments of the present disclosure also include use of a hydrogel-based composition comprising at least one therapeutic agent for the treatment and/or prevention of a cardiac injury. Embodiments of the present disclosure also include use of a hydrogel-based composition comprising at least one therapeutic agent for the manufacture of a medicament to treat and/or prevent a cardiac injury.

In some embodiments, the hydrogel component comprises at least one of a hyaluronic acid (HA)-based hydrogel component, a decellularized extracellular matrix (ECM) hydrogel component, a polyvinyl alcohol (PVA)-based hydrogel component, and any combinations or derivatives thereof. In some embodiments, the at least one therapeutic agent comprises a growth factor, a microRNA, a microRNA mimic, an exosome, a stem cell, and any combinations or derivatives thereof. In some embodiments, the at least one therapeutic agent comprises Fibroblast Growth Factor (FGF), and wherein the hydrogel component comprises a polyvinyl alcohol (PVA)-based hydrogel component.

In some embodiments, the hydrogel-based composition further comprises N¹-(4-boronobenzyl)-N³-(4-boronophenyl)-N¹,N¹,N³,N³-tetramethylpropane-1,3-diaminium (TSPBA), and wherein exposure of the composition to reactive oxygen species (ROS) cleaves the TSPBA from the PVA-based hydrogel component and releases the at least one therapeutic agent. In some embodiments, the concentration of PVA ranges from about 7% to about 11% of the composition, and wherein the concentration of TSPBA ranges from about 1% to about 5% of the composition.

In some embodiments, the at least one therapeutic agent comprises miR-21, miR-125, miR-146, or any combination thereof, and wherein the hydrogel component comprises a decellularized extracellular matrix (ECM) hydrogel component. In some embodiments, the miR-21, miR-125, miR-146, or any combination thereof, is present in the composition at a concentration ranging from about 2 nM to about 2 μM. In some embodiments, the miR-21, miR-125, miR-146, or any combination thereof, is chemically modified with an HIV TAT peptide. In some embodiments, the ECM hydrogel component is present in the composition at a concentration ranging from about 5 mg/ml to about 25 mg/ml.

In some embodiments, the at least one therapeutic agent comprises a mesenchymal stem cell (MSC)-derived exosome, and wherein the hydrogel component comprises a hyaluronic acid (HA)-based hydrogel component. In some embodiments, the HA-based hydrogel component comprises methacrylic anhydride (MA) cross-linked to HA. In some embodiments, the at least one therapeutic agent comprises a mesenchymal stem cell (MSC), and wherein the hydrogel component comprises a decellularized extracellular matrix (ECM) hydrogel component. In some embodiments, the at least one therapeutic agent comprises an induced pluripotent stem cell-derived cardiac progenitor cell (iPS-CPCs), and wherein the hydrogel component comprises a decellularized extracellular matrix (ECM) hydrogel component.

In some embodiments, the composition further comprises at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the nanovesicles, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered nanovesicle and any corresponding cargo (e.g., vaccine). Adjusting the dose to achieve maximal efficacy based on the methods described above and other methods as are well-known in the art is well within the capabilities of the ordinarily skilled person in the art.

A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the nanovesicles, a therapeutically effective amount can be initially determined from animal models. A therapeutically effective dose can also be determined from human data which are known to exhibit similar pharmacological activities, such as other adjuvants. Higher doses may be required for parenteral administration. The applied dose can be adjusted based on the relative bioavailability and potency of the administered nanovesicle and any corresponding cargo (e.g., vaccine). Adjusting the dose to achieve maximal efficacy based on the methods described above and other methods as are well-known in the art is well within the capabilities of the ordinarily skilled person in the art.

The various compositions of the present disclosure provide dosage forms, formulations, and methods that confer advantages and/or beneficial pharmacokinetic profiles. A composition of the disclosure can be utilized in dosage forms in pure or substantially pure form, in the form of its pharmaceutically acceptable salts, and also in other forms including anhydrous or hydrated forms. A beneficial pharmacokinetic profile may be obtained by administering a formulation or dosage form suitable for once, twice a day, or three times a day, or more administration comprising one or more composition of the disclosure present in an amount sufficient to provide the required concentration or dose of the composition to an environment of use to treat a disease disclosed herein, in particular a cancer.

A medicament or treatment of the disclosure may comprise a unit dosage of at least one composition of the disclosure to provide therapeutic effects. A “unit dosage or “dosage unit” refers to a unitary (e.g., a single dose), which is capable of being administered to a patient, and which may be readily handled and packed, remaining as a physically and chemically stable unit dose comprising either the active agents as such or a mixture with one or more solid or liquid pharmaceutical excipients, carriers, or vehicles.

### 3. MATERIALS AND METHODS

Preparation of ECM hydrogel. ECM hydrogel was prepared accordingly. Briefly, heart tissues were cut into pieces of 2 mm in thickness, and rinsed with deionized water (DI) water. Decellularization was performed by immersing tissues in 1% SDS in PBS for 4-5 days, until the tissue was white, then the tissues were placed in 1% Triton X-100 and stir for 30 min for final cell removal. After that, the decellularized heart tissues were washed with DI water for more than 24 hours to remove detergents. To produce the ECM hydrogel, the decellularized ECM was lyophilized and milled into a fine powder. After that, enzymatic digestion was performed using pepsin dissolved in 0.1M HCl for at least 48 hour (pepsin—matrix ratio at 1:10) with continuous stirring during the digestion. Finally, the pH was adjusted to 7.4 with NaOH on ice and diluted the ECM solution to 6 mg/mL. Gelling could occur at 37° C. water bath. Successful decellularization was confirmed with H&E staining after cryo-sectioning.

Induced pluripotent stem cells derived cardiac progenitor cell (IPS-CPC) culture. iPS-CPCs were purchased from STEMCELL Technologies (iCell® Cardiac Progenitor Cells, 01279). To trace the iPS-CPCs in vivo, GFP transfection of iPS-CPC was performed using a transfection kit (Vigene Biosciences, CV10009).

Preparation of MA-HA hydrogel. MA-HA hydrogel was prepared as previous described. Briefly, 0.1 g HA was dissolved in 10 mL deionized water (DI) water, and stirred for 30 min. After that, 2 mL of 1N NaOH as well as 0.5 mL methacrylic anhydride (MA) was added into the solution and stirred for another 2 hours. After that the mixture was placed at 4° C. for 24 hours, followed by precipitation and purification with 95% ethanol. The lyophilized powder was then dissolved in pure water and dialyzed with a 12 kDa cellulose bag. Gelatin could occur with UV irradiation at a power of 4.5 mW/cm² for 10 seconds.

MSC culture and isolation of exosomes. Mesenchymal stem cells were purchased from the American Type Culture Collection (ATCC, VA, USA). After 3 passages, MSCs were cultured in serum-free Iscove's Modified Dulbecco's Medium (IMDM) for 48 hours. The conditioned medium was collected and exosomes were isolated by the ultrafiltration method with a 0.22 μm filter. Transmission electron microscopy (TEM) was performed to confirm the morphology of exosomes. For TEM, exosomes were fixed with 4% PFA and 1% glutaraldehyde at room temperature.

Rodent model of MI and iPC injection. All animal procedures were approved by Institute Animal Care and Use Committee (IACUC) of North Carolina State University. Rodent model of myocardial infarction was induced as previously described. Briefly, the animal was anesthetized through IP injection of Ketamine-Xylazine (KX) at a dose of 100 mg/kg and 5 mg/kg respectively, followed by ventilation, and thoracotomy. Then the left anterior descending (LAD) coronary artery was ligated with a 6-0 suture while the pericardium was preserved. Infarction was confirmed by a pale color of the apex area. Immediately after MI, hydrogels with or without therapeutics were injected carefully into the pericardial cavity. The injection volumes were 100 μL (iPS-CPC, rat) or 20 μL (Exosomes, mouse) respectively. As controls, intramyocardial injection of 20 μL were also performed in a single site located near the infarct zone. After injection, the chest was closed and the animal was allowed to recovery.

Exosomes labelling and live imaging. To trace the in vivo biodistribution of exosomes, 10 μM DiD (Thermo Fisher Scientific, V22887) was used to label the exosomes. Exosomes in 20 μL HA-hydrogel was intramyocardially injected as controls. The total exosome dose was 10 mg/mL in terms of the protein concentration.

Cardiac function assessment. Cardiac function was measured at indicated time points. After anesthesia with inhalation of isoflurane, the animals were fixed to the operating plate with the body temperature maintained at 37° C. Then the M-mode cardiac movement was observed and recorded with an echo machine equipped with a 40 MHz transducer (Prospect T1, S-Sharp, Taiwan). Left ventricular dimensions at both diastole (LVIDd) and systole (LVIDs) were measured, and accordingly, the values of ejection fraction (EF), fraction shortening (FS), and LV volume at end diastole (EDV) and systole (ESV) were calculated. Five continuous cardiac cycles were collected for each animal.

Histological analysis. At indicated time points, animals were scarified with inhalation of CO₂, followed by intraventricular perfusion of chilled saline and 4% PFA. With carefully dissection, the heart wrapped with pericardium was harvested and then immersed in 4% PFA overnight. After washing with PBS for twice, the heart was placed into 30% glucose, followed by embedding with OCT and cryosectioning. A series of sections in 5 μm thickness were collected and stored at −20° C. until use. H & E staining and Masson's trichrome staining were performed by following a standard protocol.

Immunocytochemistry. Cells were fixed with 4% paraformaldehyde (PFA) for 15 min at room temperature (RT), followed by PBS washing twice. Then the blocking serum was added and incubated at RT for 1 hour to block the non-specific staining. After that the primary antibody (Ki67, α-SA) working solution was added and incubated overnight at 4° C. After washing with PBS, the corresponding secondary antibody was incubated. DAPI was used to stain the nucleus. TUNEL staining was introduced by using commercial labelling kit (Promega, G3250), and after reaction, α-SA staining was performed.

Intrapericardial injection in pigs. Male pigs (20-30 kg) were sedated with TKX cocktail (1 mL/13-30 kg IM). An ear venous catheter was placed once unconscious, and anesthesia was induced with isoflurane (up to 5% by mask). Then, the animal was intubated, and anesthesia was maintained with a mixture of isoflurane (2% in 100% oxygen). Sterile techniques were performed including sterile instruments, gloves, cap and mask, sterile preparation of the skin and techniques to maintain sterility of the instruments during surgery. The pigs were placed in a supine position with a 30-degree inclination on the right side and left chest was used for port access. Local anesthesia was provided at the port sites by using an infusion of lidocaine or bupivacaine (1 mg/kg-2 mg/kg). Two 10 mm ports introduced with trocars were used for iPC injection needle and camera port. the camera port was placed in the 3rd intercostal space at the level of scapula angle, while the injection port was placed in the 7th intercostal space at the posterior axillary line. After having access to the intrapericardial cavity through the port, treatments were given through intrapericardial injection by using a 15 cm introducer needle. The injection volume was 6 mL for each pig. After injection, the incision was closed.

iPC procedures in a human patient. The patient was undergoing a LARIAT procedure, which is a minimally invasive, nonsurgical procedure that helps prevent stroke in patients with atrial fibrillation (AFib) who are unable to take blood thinning medication. This was a clinically necessary procedure for the patient, and no unapproved or off-label drugs were introduced during the procedure; therefore no additional IRB approval was needed. Nevertheless, the procedure demonstrated the feasibility of minimally invasive iPC access which could be later on used for injection of therapeutics. Briefly, a lateral view angiogram was obtained under fluoroscope. After that, using a small bore (0.018″) access needle, iodinated contrast was injected to visualize the border of the pericardial space. A wire was advanced into the pericardial space. Next, a series of dilations were performed prior to the introduction of the access sheath in this case or foreseeable a catheter can be advanced here for intrapericardial injection

Blood test in pigs. Before and three days after injection, blood samples were drawn and blood test was performed by the Department of Clinical Pathology, College of Veterinary Medicine, NC State University.

Pericardial fluid collection and inflammation assay. Pericardial fluid was collected before and 3 days after injection, and the levels of inflammatory cytokines in the pericardial fluid was measured by using the Porcine Cytokine Array (Raybiotech Inc, C1 Kit).

Antibodies. Antibodies against Ki67 (ab16667, Abcam), α-Sarcomeric Actinin (SA, ab9465, Abcam), vWF (ab6994, Abcam), CD31 (ab28364, Abcam), Podoplanin (ab10288, Abcam), Vimentin (ab92547, Abcam), Sca-1 (ab109211, Abcam), α-SMA (ab32575, Abcam), MPO (PA5-16672, Thermo Fisher), CD4 (ab237722, Abcam), CD8 (ab33786, Abcam), cTnT (MS-295P, Invitrogen), Nkx2.5 (ab106923, Abcam) as well as Alexa Fluo 594 or 488 conjugated Goat anti Rabbit or mouse secondary antibodies were purchased from Abcam. TUNEL staining kit was purchased from Promega (G3250).

Images acquisition and statistical analysis. Animals were randomized to treatment groups. Statistical analysis was performed using GraphPad Prism 7 and data was expressed as mean±SD. Comparisons between two groups were performed with 2-sided Student t-test, while for multiple groups comparison, one-way ANOVA was used with Bonferroni post correction. p<0.05 was used as the criterion for significance.

Cell culture and lentiviral transduction. In vitro, the GFP-MSCs were cultured in IMDM (Invitrogen, Carlsbad, CA, USA) containing 10% fetal bovine serum (FBS) (Corning, Corning, NY, USA) in T175 tissue-culture flasks (Corning). Cells were washed with PBS and passaged with TrypLE Select (Life Technologies, Carlsbad, CA, USA). All cultures were incubated in 5% CO₂ at 37° C. CD63-RFP lentiviruses were used to transduce MSCs at MOI=20. Briefly, 50,000 cells were seeded in each well of a 24-well plate in cell culture medium and allowed to reach 70% confluence before transduction. 2.5 μL of TransDux™ and 100 μL of MAX Enhancer was combined with 400 μL of culture medium at a concentration of 1× and then pipetted into each well. Finally, two types of the lentivirus of CD63-RFP and Luc-GFP was added to each well at MOI=20 (approx. 6 μL/well), respectively, and incubated at 37° C. for 72 h. After antibiotics (puromycin) selection, the transduced cells were passaged and checked using a confocal microscope (Zeiss LSM 880).

Mouse echocardiology. This methodology was taken from a previous study. 6 mice from each group were randomly selected and anesthetized with an isoflurane/oxygen mixture before undergoing transthoracic echocardiography in the supine position at 2 days, 2 weeks, and 6 weeks after the MI model was created. A veterinary cardiologist who was blinded to the experimental design performed the procedure using a high-frequency ultrasound system (Prospect, S-Sharp, New Taipei City, Taiwan) with a 40-MHz probe. The hearts were viewed in two-dimensions (2D) along the long axis, at the height of the greatest LV diameter. The following formula was used to determine the ejection fraction: EF=(LVEDV−LVESV/LVEDV)×100%; and the fractional shortening: FS=(LVEDD−LVESD/LVEDD)×100%.

Histology. This methodology was taken from a previous study. For immunohistochemistry, heart cryosections were fixed with 4% paraformaldehyde. Permeabilization and protein blocking were done with protein block solution (Dako, Carpinteria, CA, USA) containing 0.1% saponin (Sigma-Aldrich, St. Louis, MO, USA). Proteins of interest in the samples were targeted with the following primary antibodies after an overnight incubation at 4° C.: rabbit anti-Ki67 (1:100; ab15580, Abcam, Cambridge, UK), mouse anti-α-Smooth Muscle Actin (α-SMA) (1:100; ab7817, Abcam), and mouse anti-Sarcomeric Alpha Actinin (α-SA) (1:100; ab9465, Abcam). Primary antibodies were conjugated with Alexa Fluor® 594 (1:200; ab150080, Abcam), or Alexa Fluor® 647 secondary antibodies (1:200; ab150115, Abcam). For the cell apoptosis assays, heart cryosections were incubated with TUNEL solution (Roche Diagnostics GmbH, Mannheim, Germany). DAPI (Life Technologies, Carlsbad, CA, USA) was used for nuclear staining. Images were taken with an Olympus epifluorescent microscope.

For H&E staining, sections were fixed in hematoxylin (Sigma-Aldrich, St. Louis, MO, USA) for 5 min at room temperature, and then rinsed for 2 min in running water. The sections were then dipped in acid alcohol for 2 s, in sodium bicarbonate (five dips), and in dehydrant (Richard-Allan Scientific, Kalamazoo, MI, USA) for 30 s. They were subsequently submerged in eosin (Sigma-Aldrich, MO, USA) for 2 min and thoroughly washed in dehydrant and xylene (VWR, Radnor, PA, USA).

ELISA assay for cell retention and exosome uptake. GFP-MSCs were first placed on Petri-dishes in vitro, to make a curve representing the relationship between cell numbers and GFP concentration in the cells. For in vivo study, animals in either the IPC or IM group were euthanized 1 week after treatment, and their organs were harvested for cell retention quantification. GFP and RFP expression in the MSCs was assessed through enzyme-linked immunosorbent assay (ELISA) kits (Abcam) as per the manufacturer's instructions.

IVIS imaging. Luciferase-MSCs were first placed on Petri-dishes in vitro, to make a curve representing the relationship between cell numbers and bioluminescence. The animals received IPC or IM injection of Luc-MSCs (n=5) or transplantations of empty ECM gel in the case of the control group (n=5). Animals were anesthetized with an isoflurane/oxygen mixture 1 week after treatment and imaged in the Xenogen IVIS Imaging System (Caliper Life Sciences, Hopkinton, MA, USA) to detect bioluminescence for a retention rate quantification.

Statistical analysis. All experiments were performed independently at least three times. Results are shown as means±standard deviation (SD). Comparisons between any two groups were performed using the two-tailed, unpaired Student's t test. Comparisons between more than two groups were performed using the one-way analysis of variance (ANOVA), followed by the post hoc Bonferroni test. Single, double, and triple asterisks represent P<0.05, 0.01, and 0.001, respectively; P<0.05 was considered statistically significant.

Materials. The 4-(bromomethyl) phenylboronic acid, polyvinyl alcohol (PVA, Mw=13000-15000), and N,N,N′,N′-tetramethyl-1,3-propanediamine (TMPA) were purchased from Sigma-Aldrich. CellROX™ deep red reagent were obtained from fisher scientific. Anti-sarcomeric alpha actinin antibody (ab9645 and ab137346), anti-Ki67 antibody (ab15580), anti-von Willebrand Factor (vWF) antibody (ab6994), anti-CD31 antibody (ab222783), goat anti-rabbit IgG H&L (Alexa Fluor 488), goat anti-rabbit IgG H&L (Alexa Fluor 594) (ab150080), goat anti-mouse IgG H&L (Alexa Fluor 488) (ab150113), goat anti-mouse IgG H&L (Alexa Fluor 594) (ab150116), and anti-CD68 antibody (ab31630) were obtained from Abcam. SD rats were purchased from Charles River Laboratories.

Production, purification, and identification of recombinant aFGF, bFGF, FGF21 and KGF2. All growth factors were expressed using the constructed plasmid in E. coli and produced as previously described. The factors were purified using HPLC and identified by mass spectrometer.

Proliferation of NRCMs with various growth factors. NRCMs were isolated from SD rats as previously described.^{[2]} NRCMs were cultured in 96-well plates for 3 d, followed by co-incubation with aFGF, bFGF, FGF21, or KGF2 for 24 h at different concentrations (0.01, 0.1, 1, 5, and 10 μM). Cell proliferation was evaluated using MTT assay. In addition, NRCMs were cultured in four-well chamber slides for 3 d, followed by co-incubation with aFGF, bFGF, FGF21 or KGF2 for 24 h (at 1 μM). Cell medium with or without 10% FBS were used as controls. After the incubation period, cells were washed with PBS twice, fixed, permeabilized, and stained for Ki67, followed by 4,6-diamidino-2-phenylindole dihydrochloride (DAPI) staining for nucleus visualization. Images were taken using an Olympus FV3000 confocal microscope (Olympus Corporation, Japan).

Synthesis of ROS-responsive N¹-(4-boronobenzyl)-N³-(4-boronophenyl)-N¹,N¹,N³,N³-tetramethylpropane-1,3-diaminium (TSPBA) linker. TSPBA was synthesized from the quaternization reaction between TMPA and 4-(bromomethyl) phenylboronic acid according to the literature.

Preparation of bFGF-loaded and ROS-responsive PVA-TSPBA gel (Gel-bFGF). PVA-TSPBA gel was prepared by mixing PVA and TSPBA. To fabricate a soft gel, 100 μL of PVA with different of wt % (3%, 6%, and 9%) and bFGF (10 wt %, 30 μL) were mixed first, followed by the addition of 30 μL of TSPBA with different wt % (3%, 6%, and 9%). Gels were imaged using scanning electron microscope (SEM) after lyophilization. To study ROS responsiveness, PVA-TSPBA gel loaded with Alexa Fluor 405 dye-labeled bFGF was placed into Micro ELISA Plate and incubated with different concentrations of ROS for 8 days and imaged at various time points. To study ROS-responsive bFGF release, Gel-bFGF was incubated with ROS (0.25 and 0.5 mM) and the released bFGF was measured using ELISA.

Effects of Gel-bFGF on the proliferation and viability of NRCMs. NRCMs were cultured in four-well chamber slides for 3 d, followed by co-incubation with Gel-bFGF (1 μM of bFGF)+0.25 mM H₂O₂ or Gel-bFGF (1 μM of bFGF) alone. After that, cells were washed with PBS twice, fixed, permeabilized, and stained for Ki67, followed by DAPI staining for nucleus visualization. Images were taken using an Olympus FV3000 confocal microscope (Olympus Corporation, Japan). For viability studies, NRCMs were cultured in 96-well plates for 3 d, followed by co-incubation at with Gel-bFGF at various concentrations. Cell viability was determined using MTT (3-(4,5-dimethylthiazol-2-yl)-2,5-diphenyltetrazolium bromide) assay. The medium were replaced by 0.5 mg/mL MTT and incubated at 37° C. for 4 h. Next, the unreacted dye was withdrawn and 0.2 mL DMSO was added to dissolve the intracellular purple formazan product into a colored solution and OD value at 570 nm were read.

Rat model of myocardial ischemia/reperfusion (I/R) injury. All animal work was compliant with the Institutional Animal Care and Use Committee (IACUC) of the North Carolina State University. Rat I/R model was induced as previously rescribed.^{[5,6]} The levels of ROS before and after I/R were determined using CellROX™ Deep Red Reagent kit according to the manufacturer's instructions.

Intrapericardial injection of Gel-bFGF and echocardiography in rats. PBS, Gel (7.6 mg/kg), bFGF alone (0.4 mg/kg), or Gel-bFGF (0.4 mg/kg of bFGF) were injected into the pericardial cavity during the 30 min ischemia. Echocardiography was performed with a Philips CX30 ultrasound system and a L15 high-frequency probe by a cardiologist blinded to animal group allocation. All animals inhaled a 1.5% isoflurane-oxygen anesthesia mixture in the supine position at the 4 hr and 4 week time points (n=5 rats per group). Hearts were imaged in 2D in long-axis views at the level of the greatest left ventricular (LV) diameter. Both end-systolic and end-diastolic (LVIDs and LVIDd) were measured and ejection fraction (EF) and fractional shortening (FS) were determined by values from LVIDs and LVIDd measurement.

Biodistribution of bFGF and bFGF-loaded gel in I/R rats. bFGF was pre-labeled with Cy5.5-NHS. Rats were euthanatized at 0, 2 d and 4 d and major organs (heart, liver, spleen, lung, kidney and thymus) were harvested for imaging using the IVIS imaging system (n=3 rat per group). In addition, hearts were frozen in OCT compound after imaging. Specimens were sectioned at 10 μm thickness from the apex to the ligation level with 100 μm intervals. To investigate if the released bFGF were bound to cardiomyocyte, bFGF was pre-labeled with Alexa Fluor™ 594 NHS Ester (Succinimidyl Ester, invitrogen™) and then loaded into the ROS-responsive gel. After intrapericardial delivery in I/R rat, hearts were harvested at day 3 and 10 μm sections were prepared from the apex to the ligation level with 100 μm intervals for histology analysis.

Heart morphometry. After the echocardiography study at 4 weeks, animals were euthanized and hearts were harvested and frozen in OCT compound. Specimens were sectioned at 10 μm thickness from the apex to the ligation level with 100 μm intervals. Masson's trichrome staining was performed as described by the manufacturer's instructions. Images were acquired with a PathScan Enabler IV slide scanner (Advanced Imaging Concepts, Princeton, NJ). From the Masson's trichrome stained images, the percentage of viable myocardium as a fraction of the scar area (infarcted size) was quantified.

Immunohistochemistry. Heart cryosections were fixed with 4% paraformaldehyde in PBS for 30 min, permeabilized, and blocked with Protein Block Solution (DAKO) containing 0.1% saponin for 1 h, at room temperature. TUNEL staining were carried out according to the manufacturer's instructions (In Situ Cell Death Detection Kit, Fluorescein, and Sigma). For other immunostaining including vWF, Ki67, CD31, and CD68, the samples were incubated overnight at 4° C. with the primary antibodies diluted in the blocking solution. Cardiomyocytes were co-stained by anti-alpha sarcomeric actin antibody. After labelling by fluorescence-tagged secondary antibodies, slides were mounted with ProLong Gold mountant with DAPI (Thermo Fisher Scientific) and viewed using an Olympus FV3000 confocal microscope (Olympus Corporation, Japan). Vessel densities were defined as vessel area/total area*100%. Four slides were stained for each group and 4 randomly selected fields from each slide (n=4) were analyzed with the NIH ImageJ software.

Intrapericardial injection of Gel-bFGF in pigs. Yorkshire castrated male pigs (median weight 16.5 kg) (n=3) were anaesthetized and then intubated and mechanically ventilated. General anaesthesia was maintained with isoflurane. Two 5 mm trocars were used for instrument and thoracoscope port, respectively, while a 10 mm trocar was used for the delivery tool. Blood and pericardial fluid were collected before and after treatment for further analysis. 3 days after treatment, hearts were harvested and sectioned for histological analysis.

Intrapericardial access in a human patient. The patient was undergoing a LARIAT procedure, which was a minimally invasive nonsurgical procedure that helped prevent stroke in patients with atrial fibrillation (AFib) who were unable to take blood thinning medication. This was a clinically necessary procedure for the patient, and no unapproved or off-label drugs were introduced during the procedure; therefore, no additional IRB approval or informed consent was needed. The images are fully de-identified. Nevertheless, the procedure demonstrated the feasibility of minimally invasive iPC access which could be later on used for injection of therapeutics. Briefly, a lateral view angiogram was obtained which revealed the location of the apex of the right ventricle. Next, using a small bore (0.018″) accessed needle, iodinated contrast was used to mark the border of the pericardial space and a needle was used to advance into the pericardial space. After entering the space with a needle, a wire was advanced into the pericardial space. Next, a serial dilations were performed prior to the introduction of the access sheath that can be used for iPC injection.

Statistical analysis. All experiments were performed independently at least three times, and the results were presented as mean±SD. Comparisons between any two groups were performed using two-tailed, unpaired Student's t-test. Comparisons among more than two groups were performed using one-way ANOVA, followed by post hoc Bonferroni test. Single, double, triple and four asterisks represent p<0.05, 0.01, 0.001, and 0.0001 respectively; p<0.05 was considered statistically significant.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Feasibility of iPC injection. All animal studies were approved by the Institutional Animal Usage and Care Committee of North Carolina State University. It was first demonstrated that iPC injection can be performed in mice and rats with open chest surgery (videos of which can be made available upon request), and mini-invasively in pigs with two small incisions (one for the injection needle and the other for the camera probe) on the chest wall (videos of which can be made available upon request), as well as in human patients under fluoroscope through a tiny puncture (videos of which can be made available upon request). Next, the efficacy and safety of iPC injection for cardiac repair was tested, using induced pluripotent stem cells-derived cardiac progenitors (iPSC-CPCs) and mesenchymal stem cells (MSCs)-derived exosomes as model therapeutics.

### Example 2

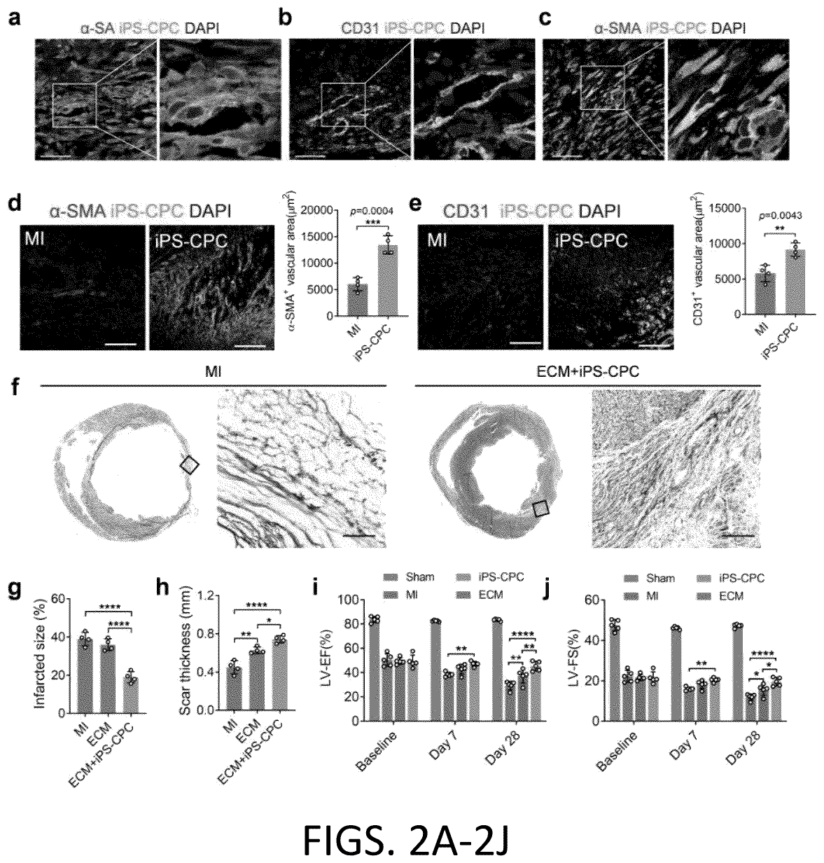

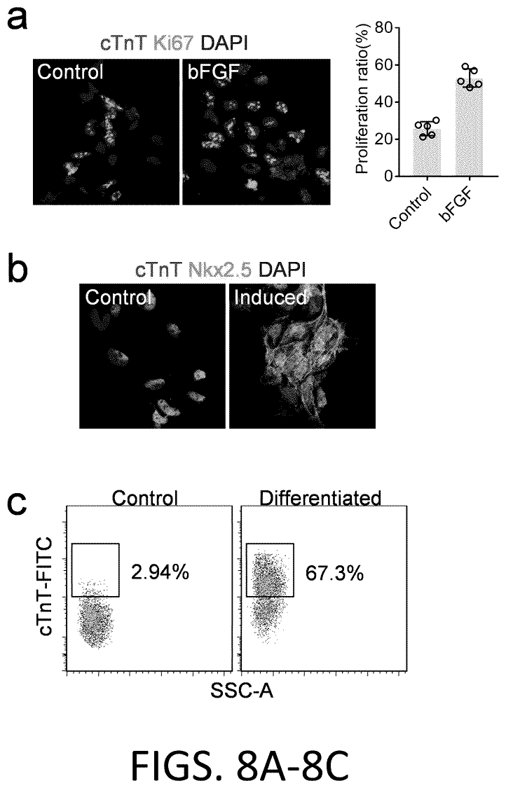

iPC injection of pluripotent stem cells causes less immune responses. The first study involved iPC injection of iPS-CPCs in an injectable decellularized extracellular matrix (ECM) hydrogel made from porcine heart. The therapy was tested in a rat model of myocardial infarction (FIG. 1). Pig heart-derived ECM was characterized (FIG. 1). Biocompatibility was confirmed after direct injection into the pericardial cavity (FIG. 2). The ability of iPS-CPCs for multi-lineage differentiation in vitro was also confirmed (FIG. 8). Emerging evidence supports the beneficial effects of iPSC therapy on ischemic heart diseases by direct differentiation and paracrine effects. However, intramyocardial (IM) injection of iPS cells can bring up risks such as teratoma formation, immune rejection, and arrhythmia. Results showed that iPC injection of iPS-CPCs in ECM hydrogel formed a cardiac patch-like structure on the infarct (FIG. 1A, B). Moreover, iPC injection overcomes the drawback of immune response, which is evident in the IM injection group (FIG. 1B, C). Infiltration of neutrophils and T cells was observed in IM-injected hearts, and this was negligible in the iPC-injected animals.

### Example 3

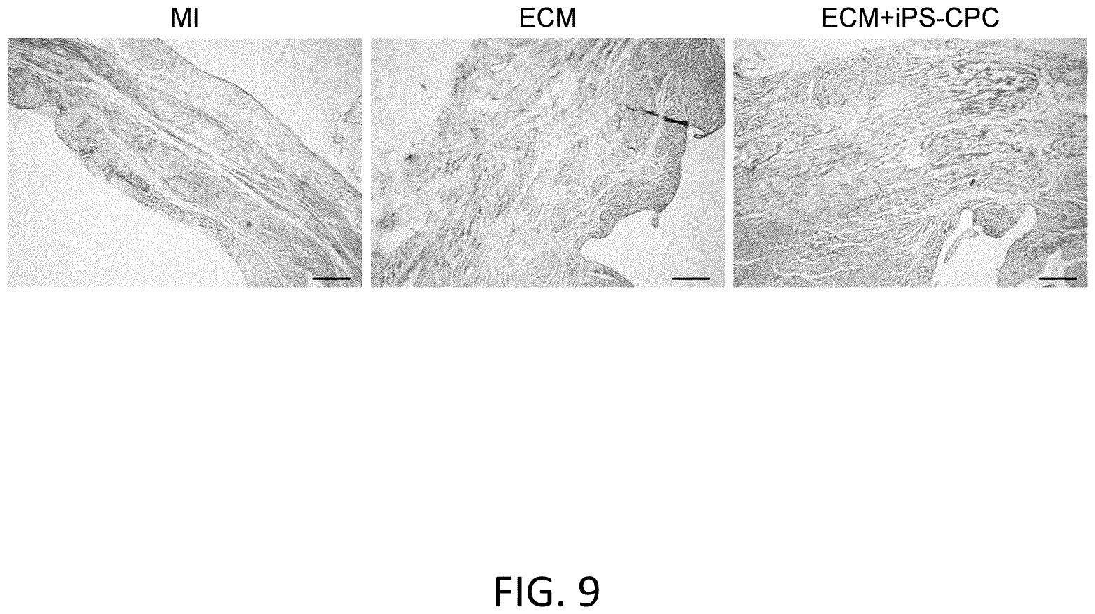

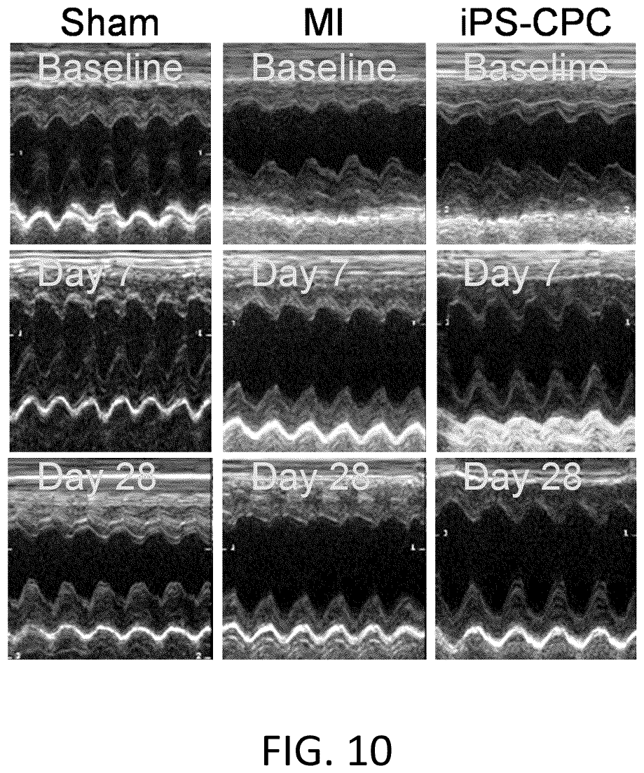

iPC delivery of stem cells contributes to cardiac regeneration and repair. Immunostaining confirmed that iPC-injected iPS-CPCs differentiated into cardiomyocytes, smooth muscle cells, and endothelial cells (FIG. 2A-C) in the post MI heart. Such direct differentiation was also accompanied by indirect paracrine mechanisms of repair. iPC injection of iPS-CPCs promoted angiogenesis (FIG. 2D, E) and reduced infarct size (FIG. 2F, G). Consistent with the improved cardiac morphology (FIG. 2H; FIG. 9), cardiac function was protected by iPS-CPCs treatment (FIG. 2I, J; FIG. 10; Table 1). Collectively, those datasets suggested that iPC delivery of iPS-CPCs in biomaterials is safe and effective for cardiac repair in a rodent model of myocardial infarction.

TABLE 1
Diameters of left ventricular at both end-diastole (LVIDd) and end-systole(LVIDs) in rats.

| | Baseline | | Day 7 | | Day 28 | |
|---|---|---|---|---|---|---|
| | LVIDd(cm) | LVIDs(cm) | LVIDd(cm) | LVIDs(cm) | LVIDd(cm) | LVIDs(cm) |
| Sham | 0.654 ± 0.017 | 0.343 ± 0.026 | 0.679 ± 0.016 | 0.366 ± 0.008 | 0.707 ± 0.007 | 0.373 ± 0.009 |
| MI | 0.648 ± 0.026 | 0.504 ± 0.006 | 0.724 ± 0.005 | 0.608 ± 0.011 | 0.845 ± 0.009 | 0.744 ± 0.007 |
| ECM | 0.658 ± 0.013 | 0.516 ± 0.017 | 0.704 ± 0.023 | 0.575 ± 0.016 | 0.775 ± 0.012 | 0.654 ± 0.012 |
| ECM + iPS-CPC | 0.659 ± 0.027 | 0.517 ± 0.032 | 0.696 ± 0.027 | 0.554 ± 0.028* | 0.729 ± 0.023** | 0.586 ± 0.006** |

Data were expressed as mean ± SD,
*p < 0.05 vs MI,
**p < 0.01 vs MI or ECM

### Example 4

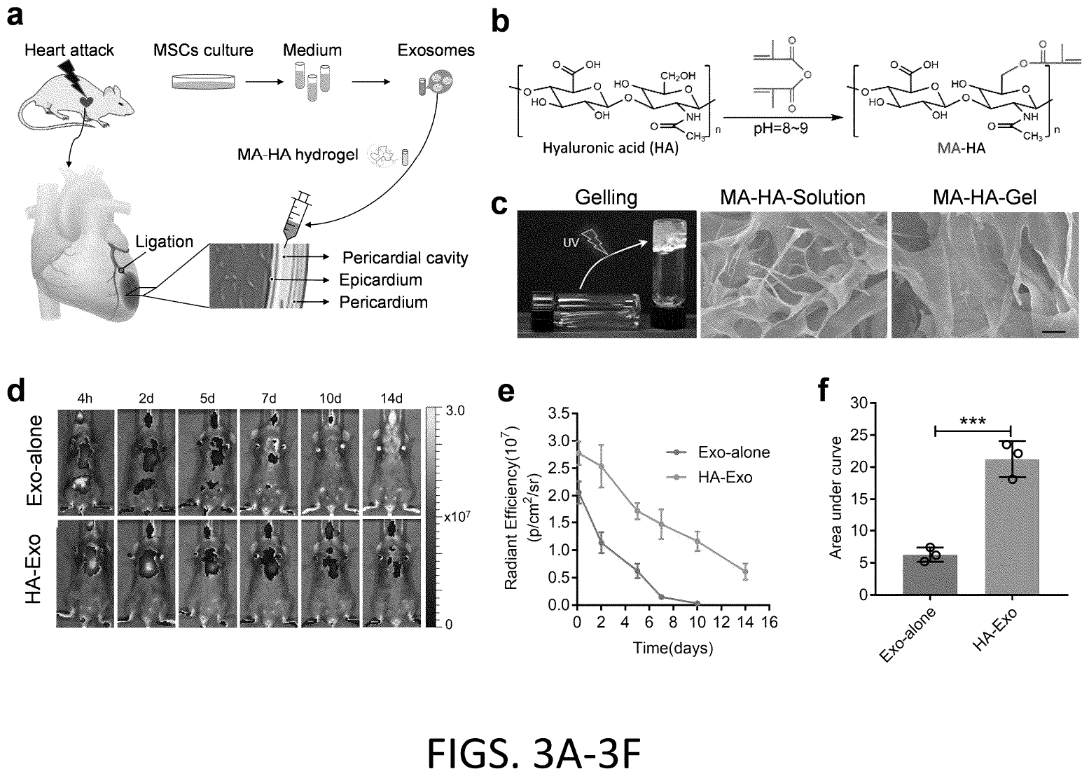

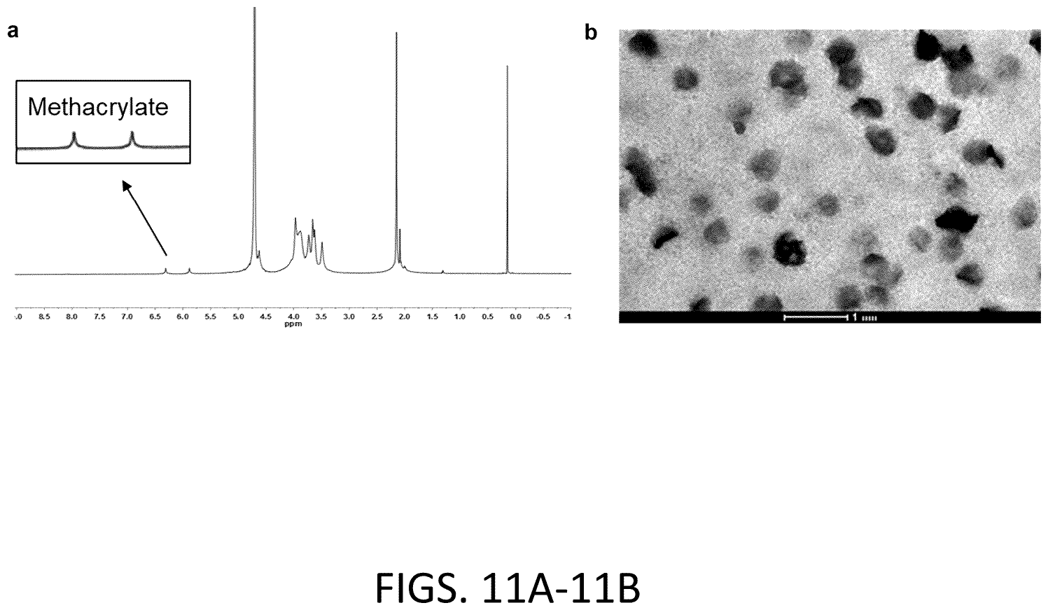

iPC injection in hydrogel enhances MSC exosome retention in the heart. The second study involves iPC delivery of therapeutic exosomes in hyaluronic acid (HA) hydrogel in a mouse model of MI (FIG. 3A). Exosomes are 30-150 nm extracellular vesicles secreted by essentially all cell types. Exosomes derived from mesenchymal stem cells (MSCs) is a promising therapeutic agent in cardiac repair. It is difficult to deliver exosomes directly to the heart. MA-HA hydrogel was synthesized by crosslinking methacrylic anhydride (MA) with HA to prepare a UV sensitive hydrogel and SEM imaging revealed the ultrastructure of the gel (FIG. 3B, C; FIG. 11A). Exosomes were derived from human MSCs using the ultracentrifugation method and the TEM image of the exosomes was shown (FIG. 11B). iPC injection resulted in a nice cardiac retention of exosomes and injection in hydrogel further prolonged the release of exosomes into the heart (FIG. 3D-F).

### Example 5

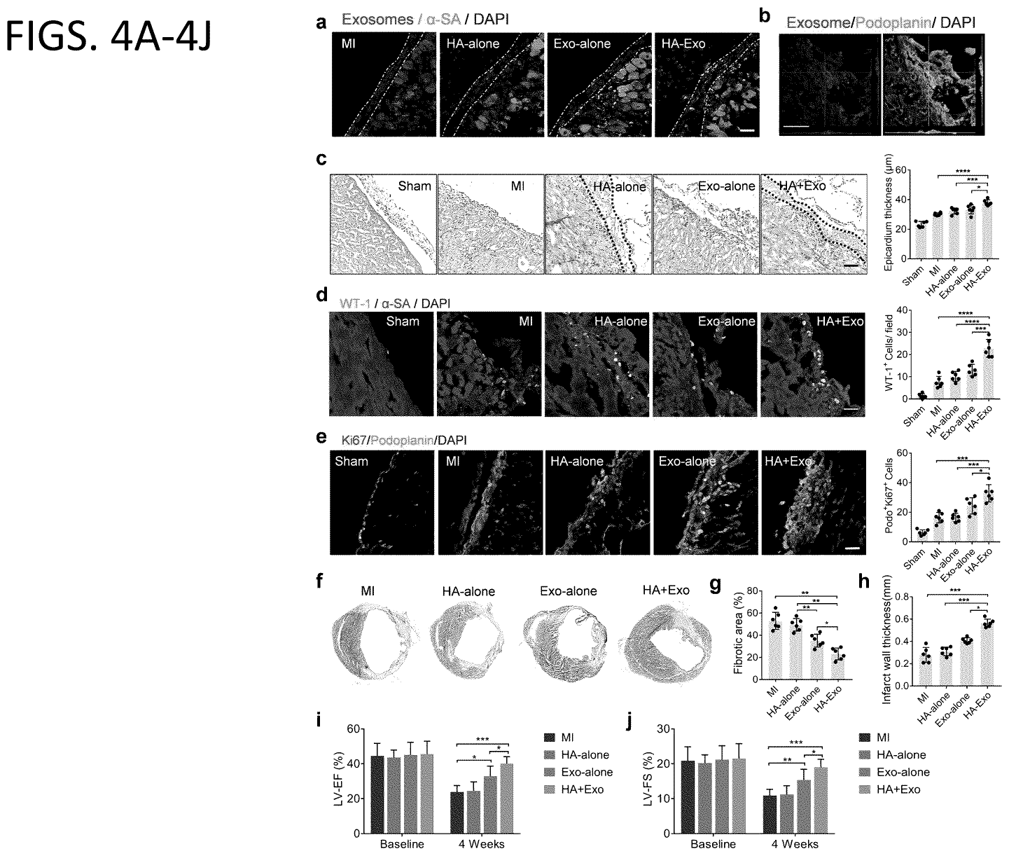

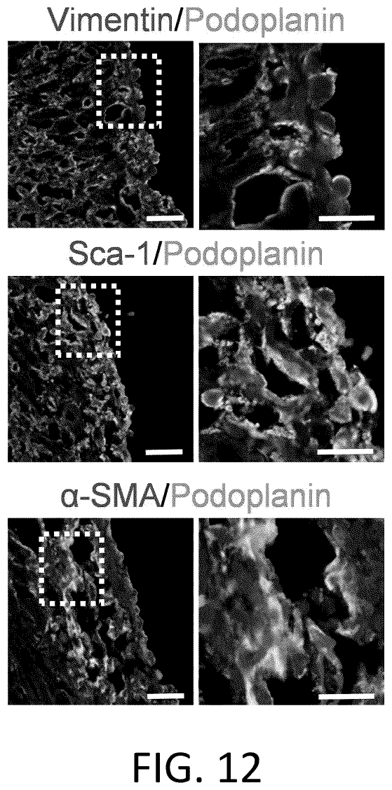

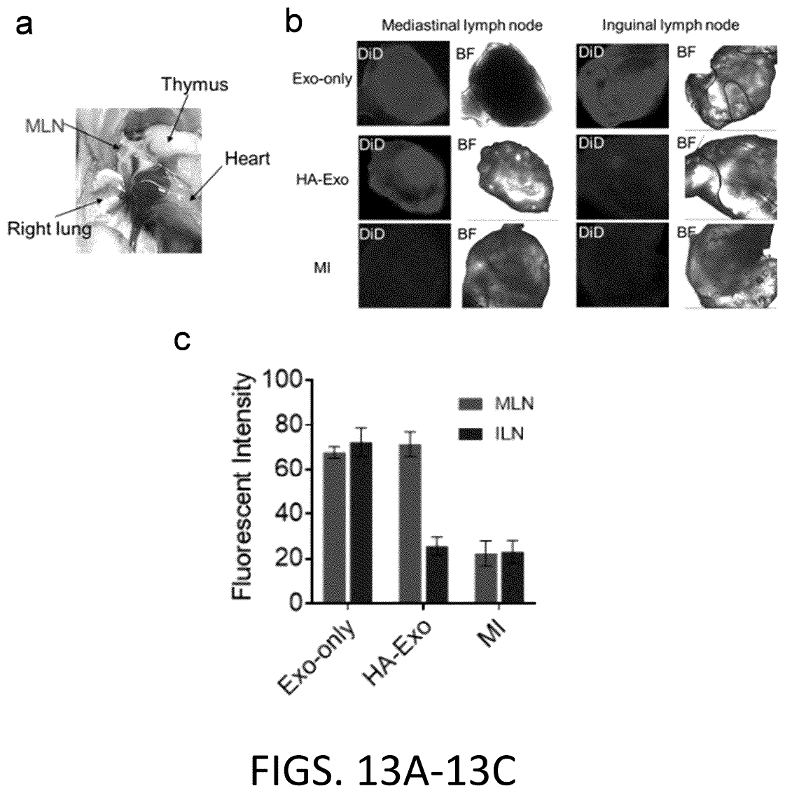

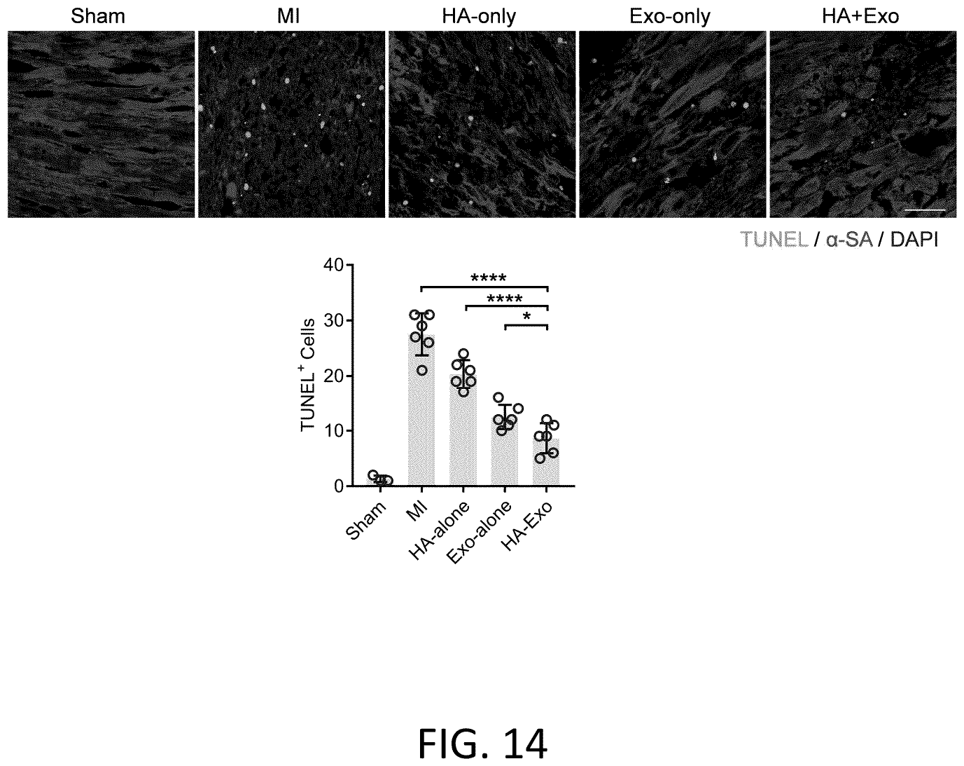

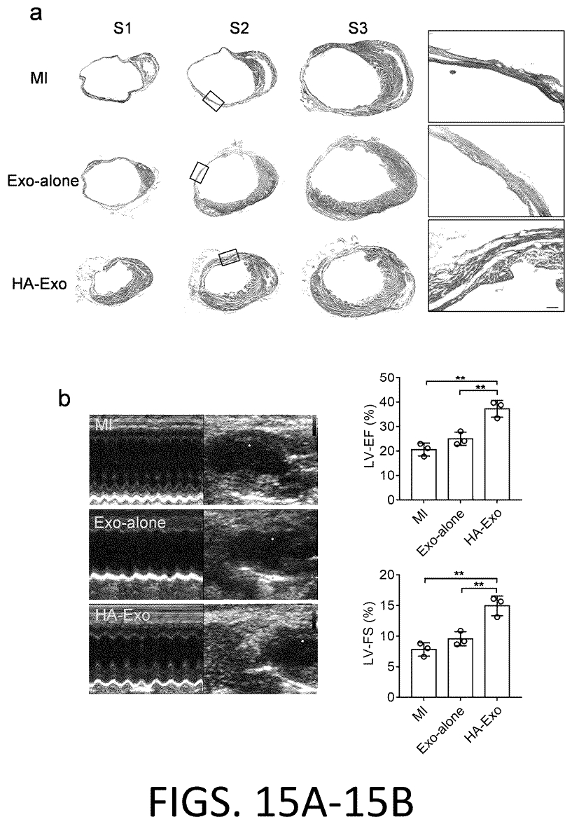

iPC delivery of exosomes promotes cardiac repair after MI. The uptake of exosomes by epicardial cells was confirmed (FIG. 4A, B), and the spreading of HA hydrogel to form a cardiac patch in the pericardial cavity (FIG. 4C). iPC injection of exosomes increased the thickness of epicardium (FIG. 4C). iPC injection of MSC-exosomes promoted the proliferation and differentiation of epicardial-derived cells (EPDCs) (FIGS. 4D, E; FIG. 12). Moreover, a significant accumulation of exosomes in the mediastinal lymph node was detected (FIG. 13). Masson's trichrome staining revealed that iPC injection of HA+Exo reduced fibrotic area in the post-MI heart (FIG. 4F, G). In addition, there was a reduction of apoptotic cells in the HA+Exo treated hearts (FIG. 14). Consistent with the improved cardiac morphology (FIG. 4F, H), echocardiography measurement demonstrated that iPC injection of HA+Exo therapy boosted cardiac functions (FIG. 4I, J). Furthermore, in the long-term detection, iPC injection of exosomes with HA hydrogel improved cardiac histology and suppressed the transition of heart failure (FIG. 15). Collectively, those datasets suggested that iPC delivery of therapeutic exosomes in biomaterials is safe and effective for cardiac repair.

### Example 6

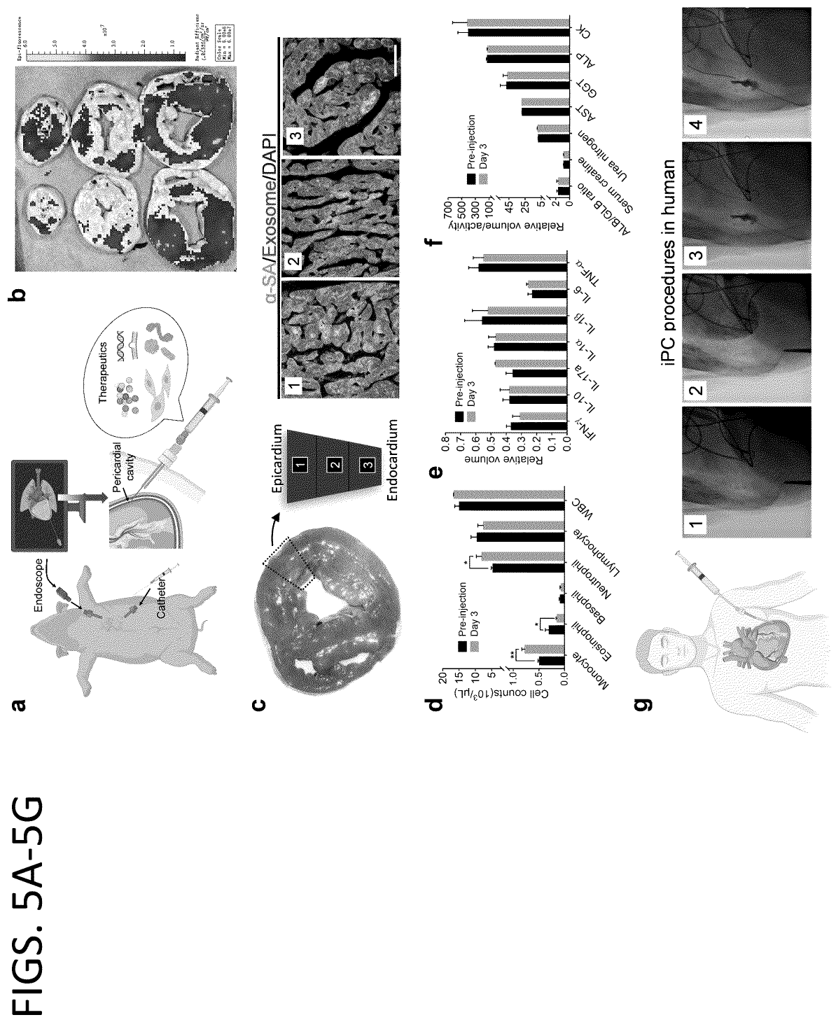

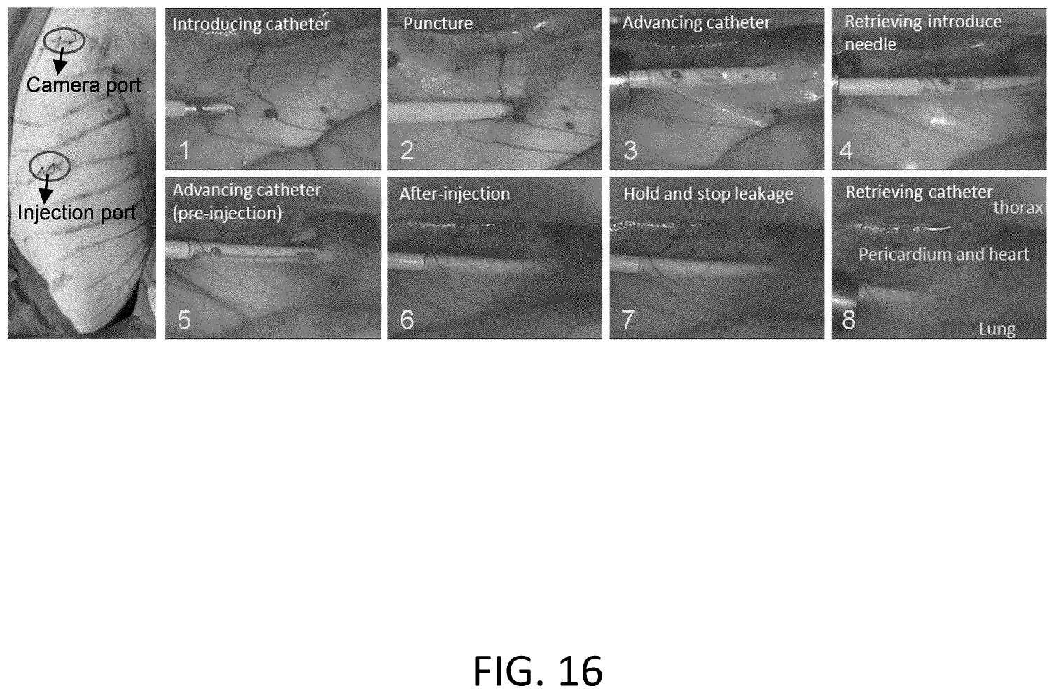

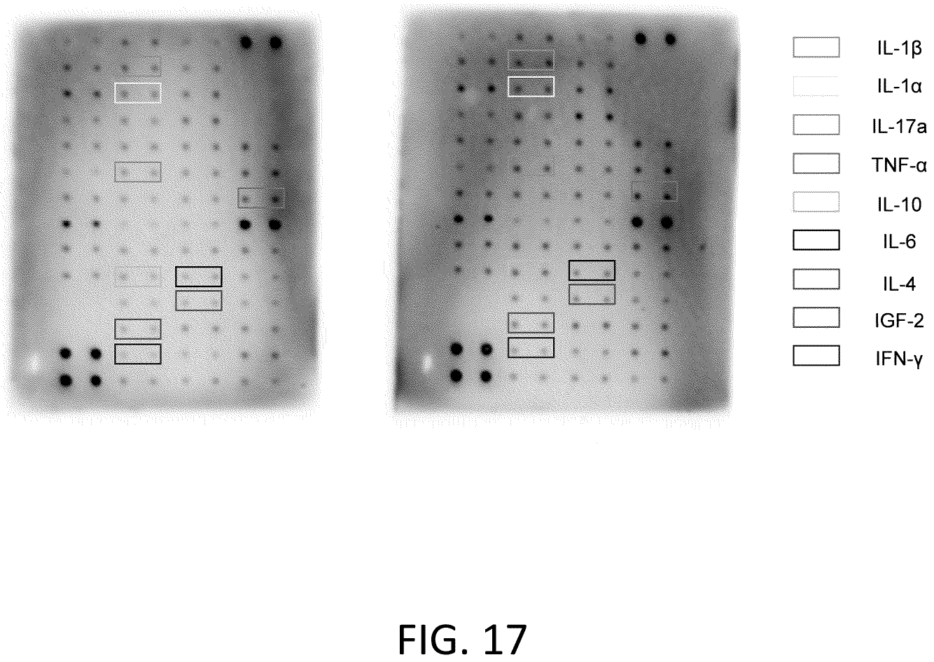

Minimally-invasive iPC injection in pigs. The third study tested the feasibility of iPC injection of therapeutics in a porcine model. The injection was enabled by two small incisions on the chest wall, one for the injection catheter and the other for the endoscope (FIG. 5A; FIG. 16). HA+Exo was used as the model therapy here. Ex vivo fluorescent imaging revealed that iPC injection led to a sizable exosome retention on the heart (FIG. 5bB). Histology further confirmed the release and uptake of exosomes by cardiomyocytes, in a wide range from the epicardium to the endocardium (FIG. 5C). The safety of iPC injection in pigs was further confirmed. Three days after the injection, a slight change of cell counts of monocyte, eosinophil and neutrophil in the blood was observed, which could be caused by the surgery procedures (FIG. 5D), since differences for the inflammation assay performed with pericardial fluid were not measured (FIG. 5E; FIG. 17). In addition, there was no change was observed in blood chemistry indicators (FIG. 5F). Taken together, these data proved the safety and feasibility of iPC procedures in translational trials.

### Example 7

Minimally invasive iPC procedures in human patients. Moreover, iPC injection can be performed in clinic patients under fluoroscope with only one small incision. As shown in FIG. 5G, firstly, a lateral view angiogram was obtained to reveal the location of the apex of the right ventricle, and using a small bore (0.018″) access needle, iodinated contrast was used to mark the border of the pericardial space. After entering the space with a needle, a wire was advanced into the pericardial space. Next, a serial dilations were performed prior to the introduction of the access sheath that can be used for intrapericardial injection.

Shortly after the procedures, there was no pleural effusion or breathing complications recorded. In a long term follow-up, the occurrence of tamponade, pericarditis, or any other adverse events were not detected. Taken together, above procedures make iPC injection a safe and promising manner for in situ patching up the heart with biomedical engineering therapeutics achieving cardiac repair.

TABLE 2
Comparison of various administration routes to the heart.

| | Intravenous | Intracoronary | Intramyocardial | Cardiac patch | Intrapericardial |
|---|---|---|---|---|---|
| Easy to perform | Yes | Yes, needs cath | No, requires open- | No, requires | Yes |
| | | lab and | chest Sx unless use | open-chest Sx | |
| | | interventional | NOGA-endomyo | | |
| | | cardiologist | injection | | |
| Invasiveness | Low | Low | High, unless use | High | Low |
| | | | NOGA-endomyo | | |
| | | | injection | | |
| Retention in the | None to | Low | Medium | High | High |
| heart | Low | | | | |
| Cost | Low | Medium | High | High | Low |

### Example 8

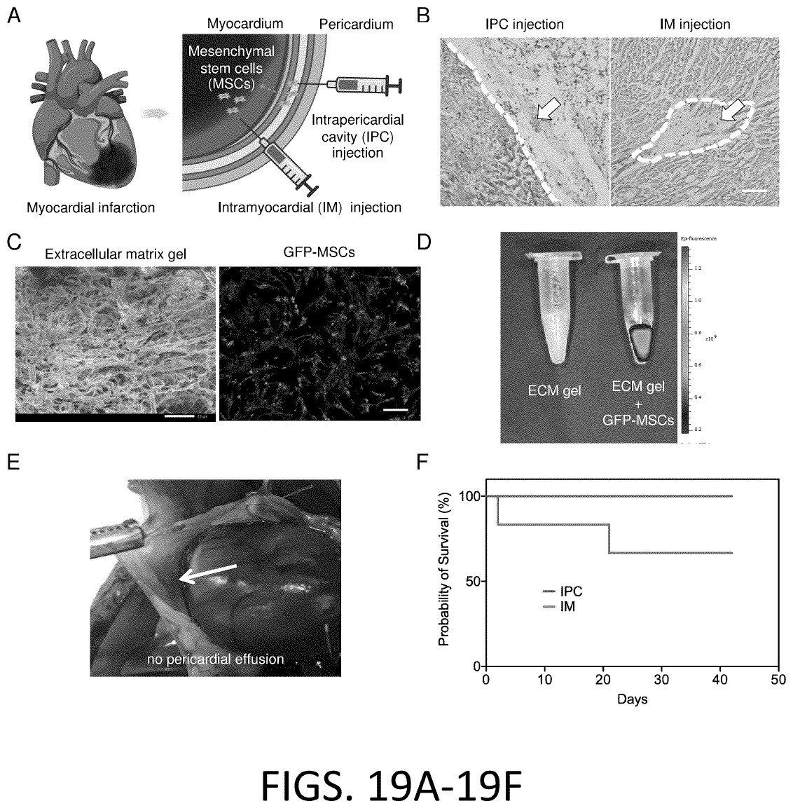

Feasibility of IPC injection in murine. The difference between two delivery routes was illustrated in a schematic image and H&E staining images (FIGS. 19A and B). The IPC method delivered the cells between the epicardium and pericardium, while the IM method delivered them to a position approximately 1 mm below the epicardium. Before the injections, 2 million GFP-MSCs were first checked for fluorescent expression in vitro under a microscope, and then suspended in ECM hydrogel (the delivery media) at a final concentration of 5,000 cells/μl (FIG. 19C). Once in the hydrogel, the cells' GFP expression was verified again with the IVIS imaging system (FIG. 19D). The complex network of the ECM gel helped protect the injected cells in the heart from being washed out rapidly once injected. The MI model in mice was constructed according to previous studies. In short, the MI model was created by ligating the left anterior descending (LAD) artery. Immediately after LAD ligation, 0.2 million GFP-MSCs (in hydrogel) were injected into the pericardial cavity (IPC delivery group, n=12) or the myocardium (IM delivery group, n=12), near the infarcted area of the mouse hearts. H&E staining was used to confirm the full extent of the injection site (FIG. 19B). Two hours after the surgery, no pericardial effusion (extra fluid around the heart) was observed in the IPC injection group (FIG. 19E), showing the safety of IPC injection method. A pericardial effusion could have led to abnormalities in the pericardial cavity and put pressure on the heart. Moreover, the overall physical condition and survival rates of the IM group were worse than that of the IPC group, with one animal dead after 1 day and another dead after 3 weeks (FIG. 19F). All the mice in the IPC group retained their physical condition and none died before their experimental endpoint.

### Example 9

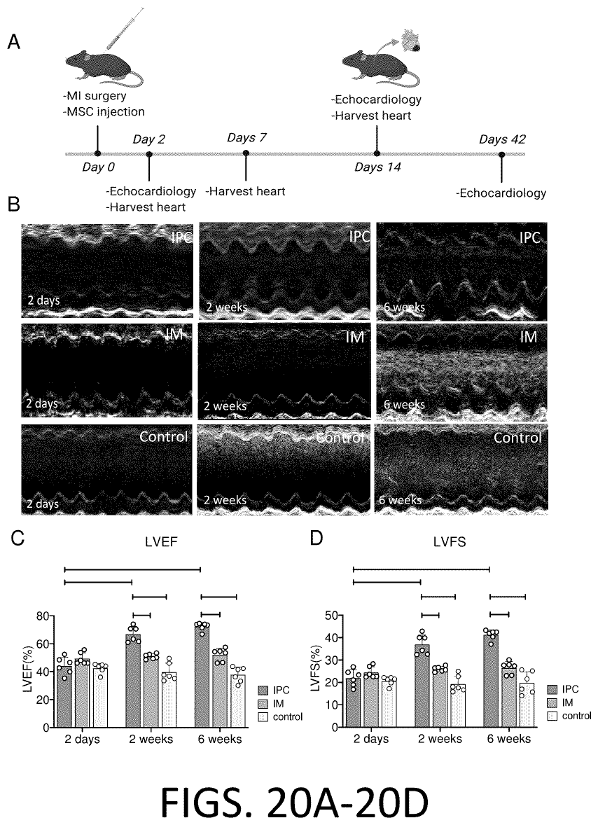

IPC delivery of MSCs improves cardiac function. All animals were examined for echocardiography 2 days, 14 days and 42 days after the surgery (FIGS. 20A and B). Two days post-injection, left ventricular ejection fraction (LVEF) and left ventricular fractional shortening (LVFS) were measured. This data is considered a baseline soon after myocardial infarction, but before the cellular intervention has had a chance to affect the heart's performance. There were no significant differences between IPC, IM, or control group (FIGS. 2C and D). At the 2-week follow-up, both the LVEF and LVFS had increased in the IPC group but not in the IM or control group (FIGS. 20C and D). After 6 weeks, the measurements had significant enhancement compared to baseline level (2 days) (FIGS. 20C and D), which showed a long-term cardiac repair after the IPC delivery of MSCs in the mouse. Furthermore, the overall left ventricular function in the IPC group was higher than that of the IM and control groups, at both the 2-week (short term) and the 6-week (long term) time points (FIGS. 20C and D). IPC delivery of MSCs improved cardiac function to a higher level than IM delivery did.

### Example 10

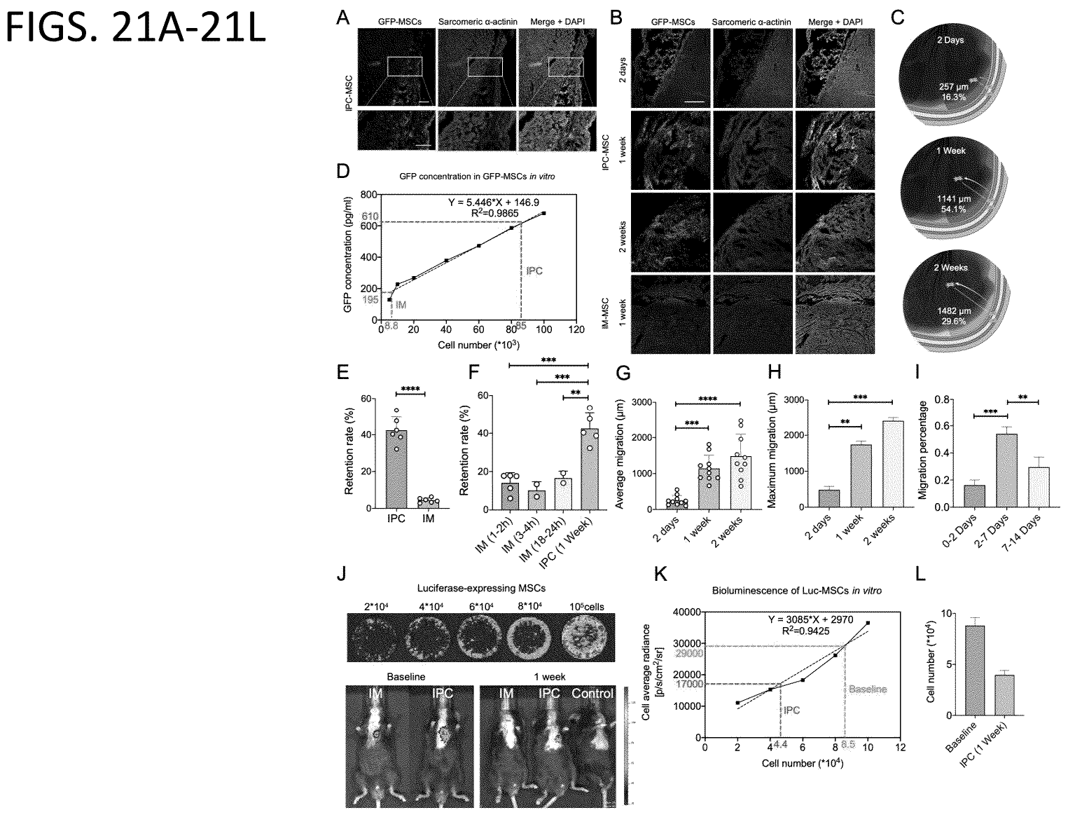

IPC delivery of MSCs yielded 10-fold higher retention than IM delivery. At 2 days, 1 week, and 2 weeks after the cell administration, the hearts were harvested in both of IPC group and IM group for immunohistochemistry (IHC) and ELISA assays (FIG. 20A). From the analysis of sarcomeric α-actinin and GFP, it was observed that the MSCs began to gradually infiltrate from the pericardial cavity into the myocardium 2 days after being administered (FIG. 21A), and for up to 2 weeks thereafter (FIGS. 21B and C). To measure the cell retention rate, GFP-MSCs were first placed on Petri-dishes in vitro, to make a curve representing the relationship between cell numbers and GFP concentration in the cells (FIG. 21D). Compared to IM injections, the IPC route resulted in higher cell retention after 1 week, which was determined via IHC and ELISA assays (FIG. 21E). Notably, in the more accurate ELISA assay, 10 times more MSC retention was found in the heart after IPC injection (42.5±7.4%) than after IM injection (4.4±1.3%) (FIG. 21E), demonstrating the ability of the pericardial cavity to keep injected cells from washing out. Additionally, the IPC injections showed an unprecedentedly high cell retention result at 1 week when compared to all other reported retention rates at any time points in previous studies (FIG. 21F). It has been confirmed that engraftment is necessary for migration to occur for such a prolonged period of time. Thus, the average and maximum distances of migration of MSCs into the myocardium were measured, and this quantification revealed a significant increase both at 1 week and 2 weeks (FIGS. 21G and H), indicating the remarkable engraftment of IPC-delivered MSCs into the ischemic heart. Interestingly, when the increase of migration distances was calculated at 3 time ranges, 0-2 days, 2-7 days and 7-14 days, a highest increase was found at 2-7 days, indicating the potentially fastest migration of IPC-injected MSCs at this period (FIGS. 21C and I). Retention rates were further confirmed with in vivo IVIS imaging. Similarly, Luciferase-MSCs were first placed on Petri-dishes in vitro, to make a curve representing the relationship between cell numbers and bioluminescence (FIGS. 21J and K). And then Luc-MSCs were injected via the IPC or IM routes into the mouse MI hearts. Mice in both groups were received in vivo IVIS live imaging immediately after injection for a baseline level and followed up at 1 week for quantification of retention rate. Bioluminescence quantification showed a cell retention rate after IPC delivery similar to that of the ELISA assay (FIGS. 21K and J). In contrast, the overall biodistribution of MSCs after IM injections could hardly be detected after 1 week (FIG. 21J). What is more, few Luc-MSCs were distributed to other organs for any unwanted accumulation (FIG. 21J), indicating the safety of the IPC delivery route in regard to biodistribution.

### Example 11

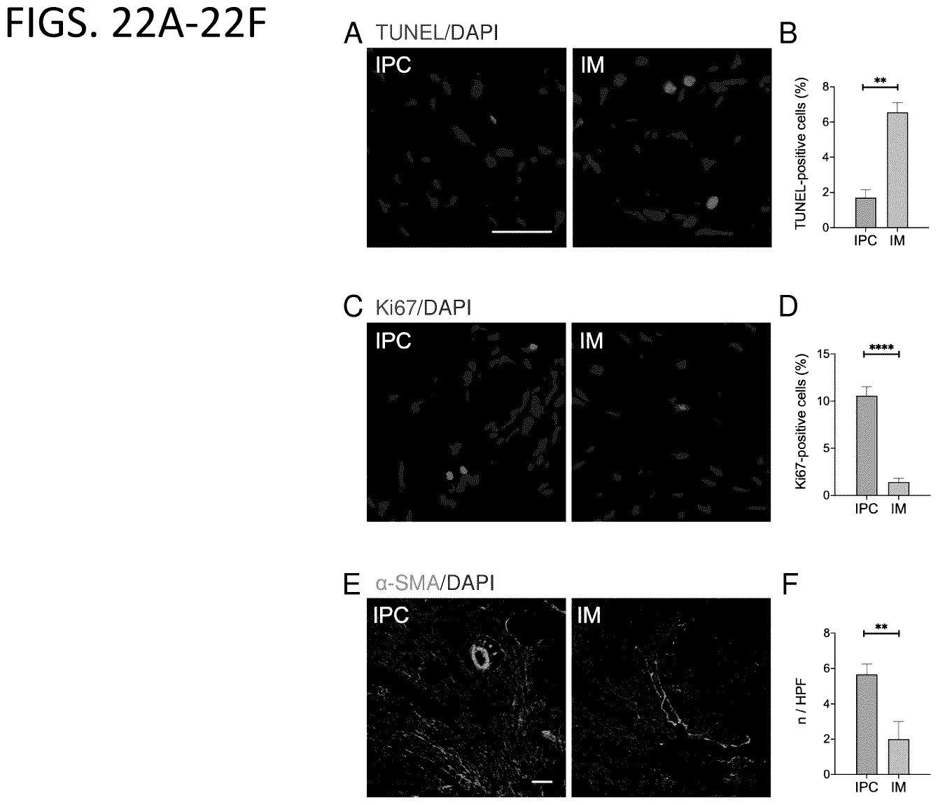

IPC delivery of MSCs leads to significant myocardial repair. After confirming that IPC-delivered MSCs improved LVEF and LVFS as yielded a higher cell retention, the heart tissues were examined histologically. Fewer TUNEL+ (terminal deoxynucleotidyl transferase-mediated deoxyuridine triphosphate nick end labeling-positive) apoptotic heart cells were found in the MI hearts after IPC delivery than after IM delivery (FIGS. 22A and B), indicating reduced myocardial apoptosis. Also, a greater number of Ki67+ cardiomyocyte nuclei where found in the MI hearts after IPC delivery (FIGS. 22C and D), demonstrating the proliferation of cardiomyocytes, which suggested enhanced cardiac regeneration. Furthermore, IPC-delivered MSCs increased vascular density in the heart after MI, which was showed by α-Smooth Muscle Actin (α-SMA) marker (FIGS. 22E and F). Together, the lack of apoptotic cells and the presence of proliferative cells in the IPC-injected group can explain the improvement of LV function at a cellular level. And the denser vasculature illustrated the cardiac regeneration at a histological level.

### Example 12

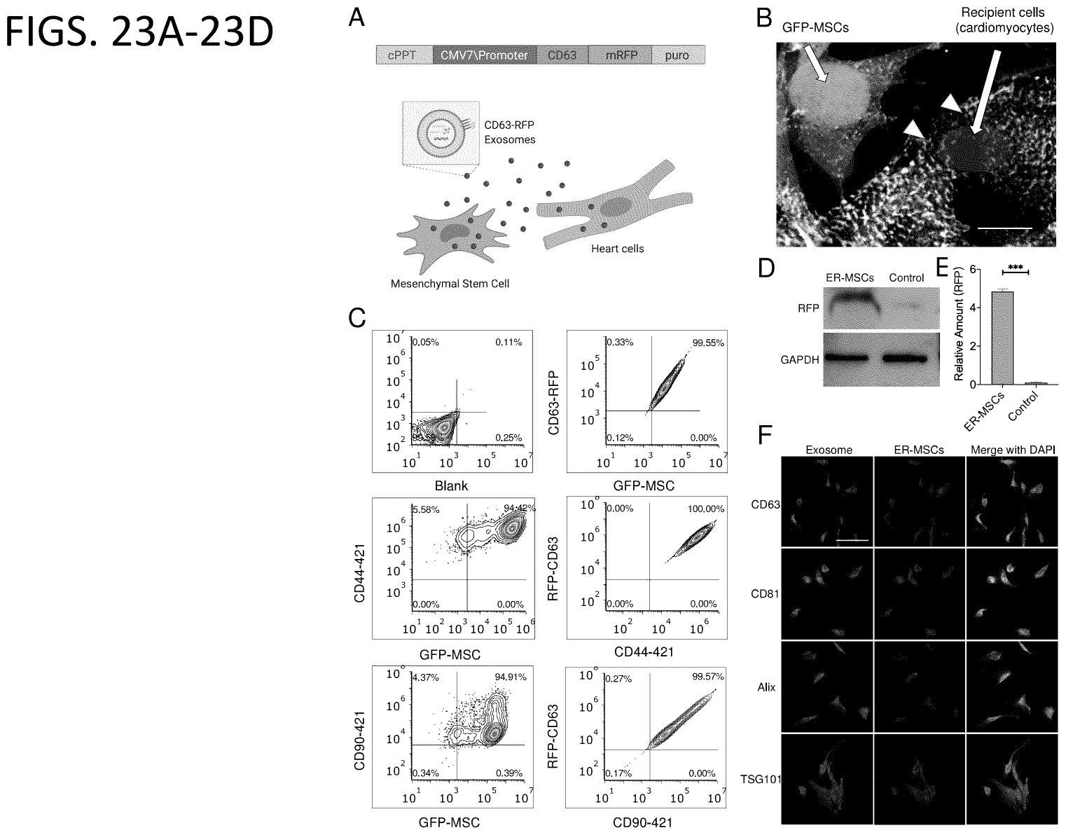

Establishment of the CD63-RFP exosome labeling system. The exosome is a type of extracellular vesicle (EV) secreted by cells that has a diameter of 100-200 nm. It plays an important role in intercellular communication and paracrine activity. Lentiviral transduction was used to genetically modify the exosomes produces by the MSCs (FIG. 23A). The transgenic MSCs would now secret exosomes expressing RFP signal, which was bound to their CD63 surface proteins and made the exosomes easy to visualize under the microscope (FIG. 23A). First, the labeling system was verified in vitro. CD63-Exo-RFP-MSCs (ER-MSCs) secreted RFP-exosomes when they were co-cultured with cardiomyocytes and RFP-exosomes were taken up by the recipient cells (FIG. 23B). In addition, the transduced CD63-Exo-RFP-MSCs (ER-MSCs) was characterized with flow cytometry (FIG. 23C) and Western Blots (FIG. 23D). ER-MSCs had significantly higher expressions of RFP compared to the control MSCs (FIG. 23E). The transduction was also confirmed by ensuring that the RFP was being emitted by vesicles that also expressed exosome-specific markers, including CD81, TSG101, and Alix (FIG. 23F). By establishing the CD63-Exo-RFP in vitro labeling system, the paracrine activity between MSCs and other cells was able to be visualized by seeing how MSC exosomes were released and absorbed.

### Example 13

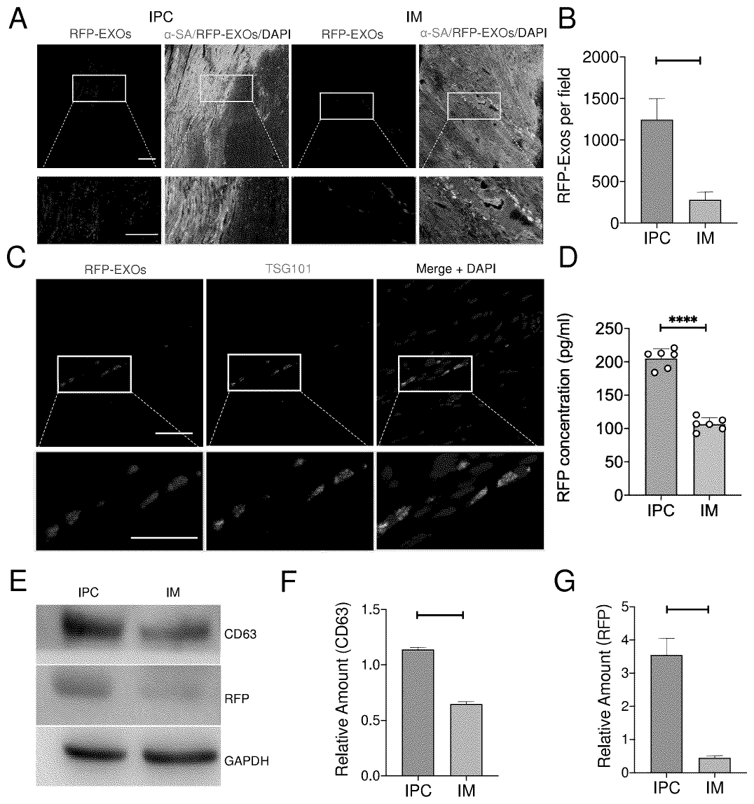

Paracrine activities of IPC-delivered MSCs. The established CD63-Exo-RFP labeling system made it possible to observe and quantify the level of cellular paracrine activity in vivo. There is now a large number of evidence supporting the hypothesis that paracrine mechanisms are crucial for tissue regeneration, and that transplanted stem cells exert their therapeutic effects by secreting biologically active proteins, or paracrine factors, to resident cells. As an important carrier for these factors, exosomes were selected to measure the MSCs' paracrine activity. ER-MSCs were injected via the IM or the IPC route in mice with induced MI. After 1 week post-injections, hearts were harvested from both groups for IHC and ELISA (RFP). More RFP-positive units were found in the IPC group than the IM group in the IHC (FIGS. 24A and B), showing a higher levels of exosome secretion activity by the IPC-injected MSCs. TSG101, another exosome specific marker, was used to identify and verify RFP-Exosomes under the microscope (FIG. 24C). In addition, ELISA and Western Blot for the expression of CD63 and RFP were used to quantify the difference between IPC and IM (FIGS. 24D and E). ELISA showed significantly higher expression of RFP after IPC delivery of ER-MSCs (FIG. 24D) and WB also showed significantly higher expression of CD63 and RFP in the IPC group, compared to IM (FIGS. 24F and G). The established CD63-Exo-RFP labeling system allowed for observing more extensive paracrine activity of MSCs delivered by IPC route, which was demonstrated by denser exosome uptake in the heart cells. These results further support that IPC-delivered MSCs augmented cardiac repair by exerting stronger beneficial effect on heart cells after myocardial infarction.

### Example 14

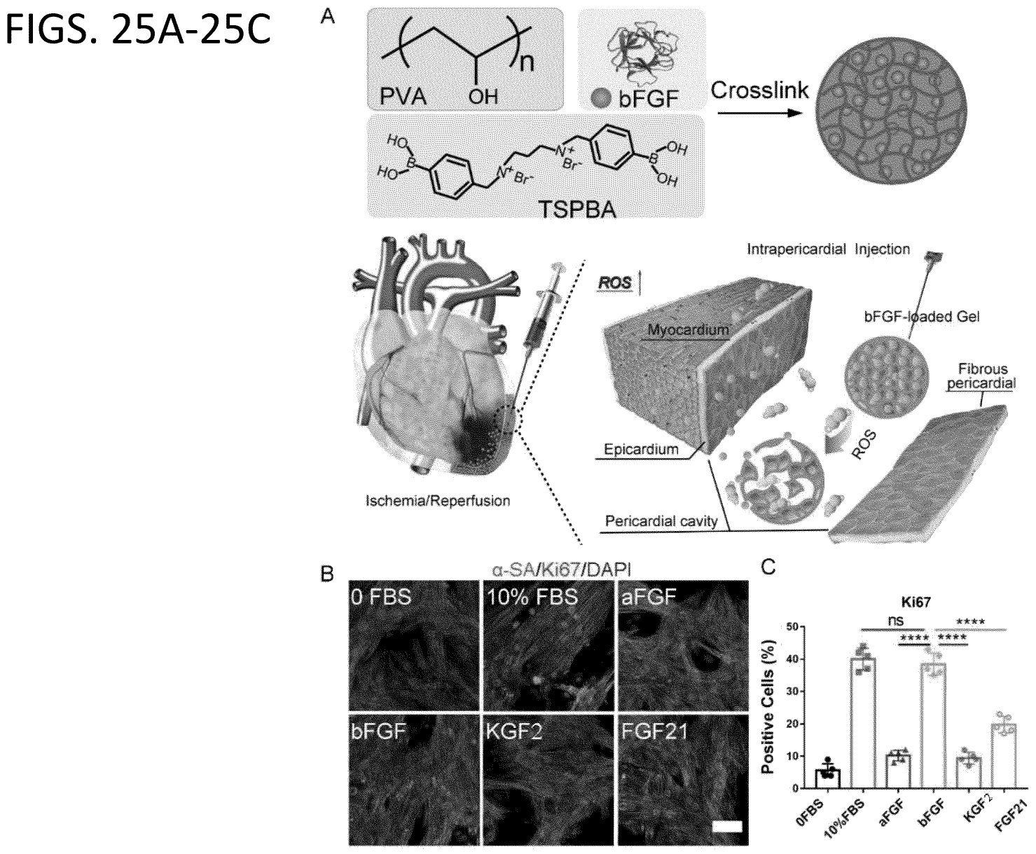

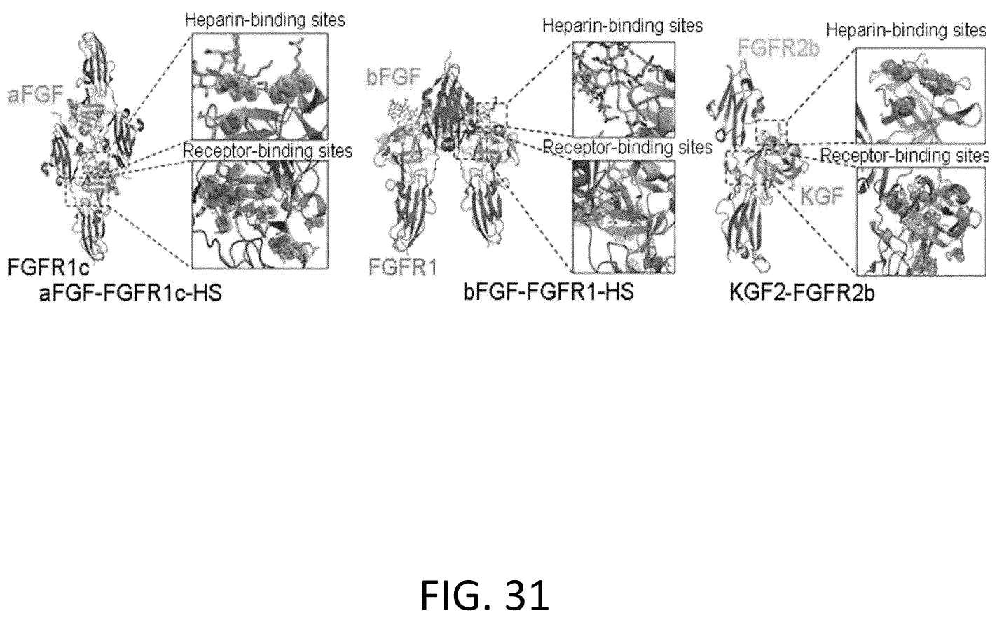

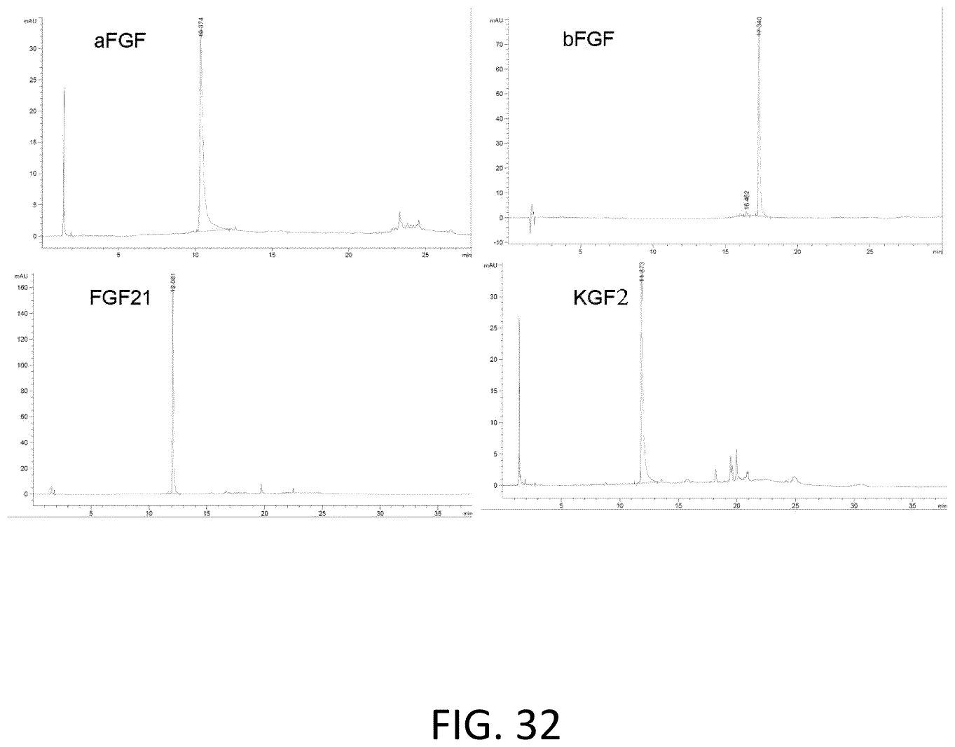

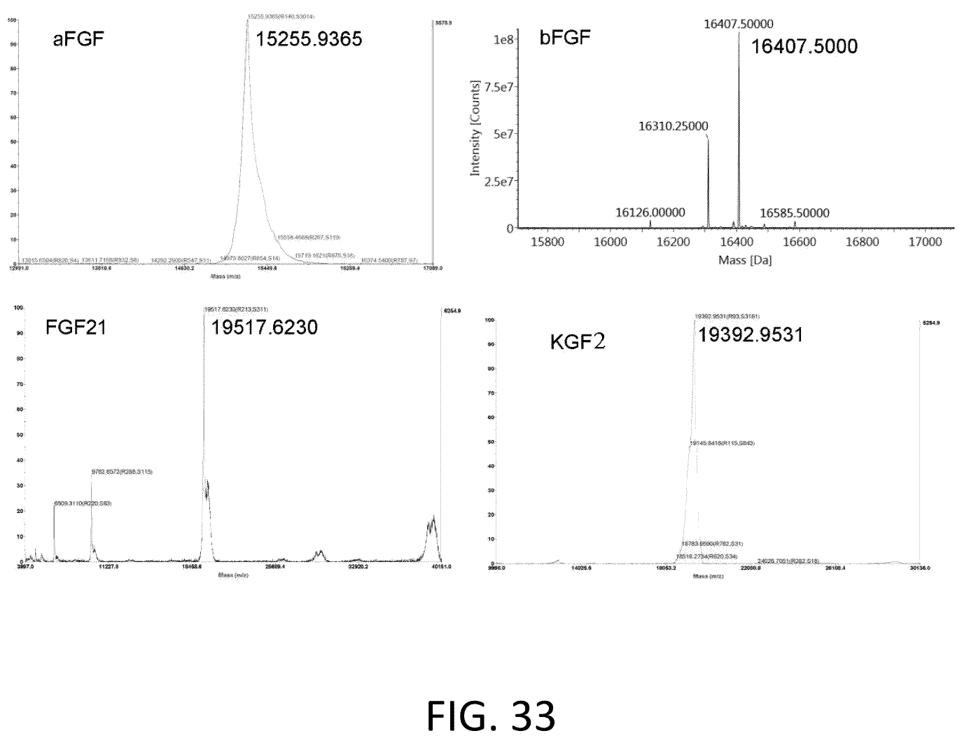

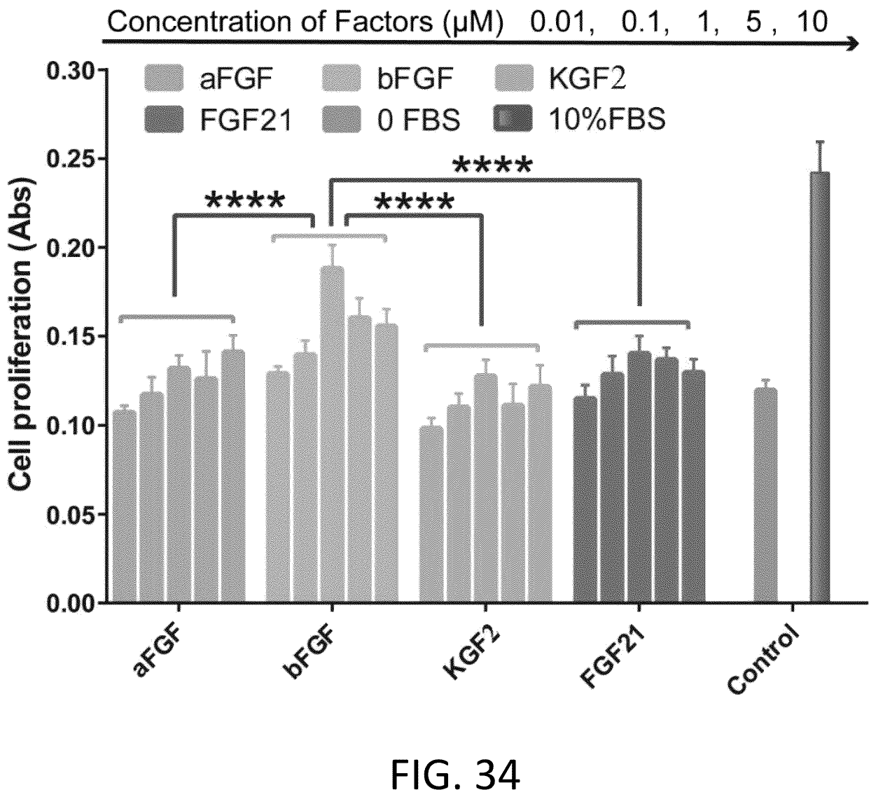

Screening of optimal FGF for heart repair. Four different types of fibroblast growth factors were produced and purified, including acid fibroblasts growth factor (aFGF, or FGF1), basic FGF (bFGF, or FGF2), FGF21 and keratinocyte growth factor 2 (KGF2, or FGF-10) (FIG. 31). High performance liquid chromatography (HPLC) confirmed the purity of the growth factors (FIG. 32). In addition, all factors were identified by mass spectrum for the expected molecular weight (FIG. 33). The effects of these growth factors on the proliferation of neonatal rat cardiomyocytes (NRCMs) were then evaluated. As shown in FIGS. 25B and 25C, bFGF showed the strongest effects on the proliferation of NRCMs, as indicated by an increase in the numbers of Ki67^{pos} cells. The concentration of bFGF was then optimized for further experiments (FIG. 34).

### Example 15

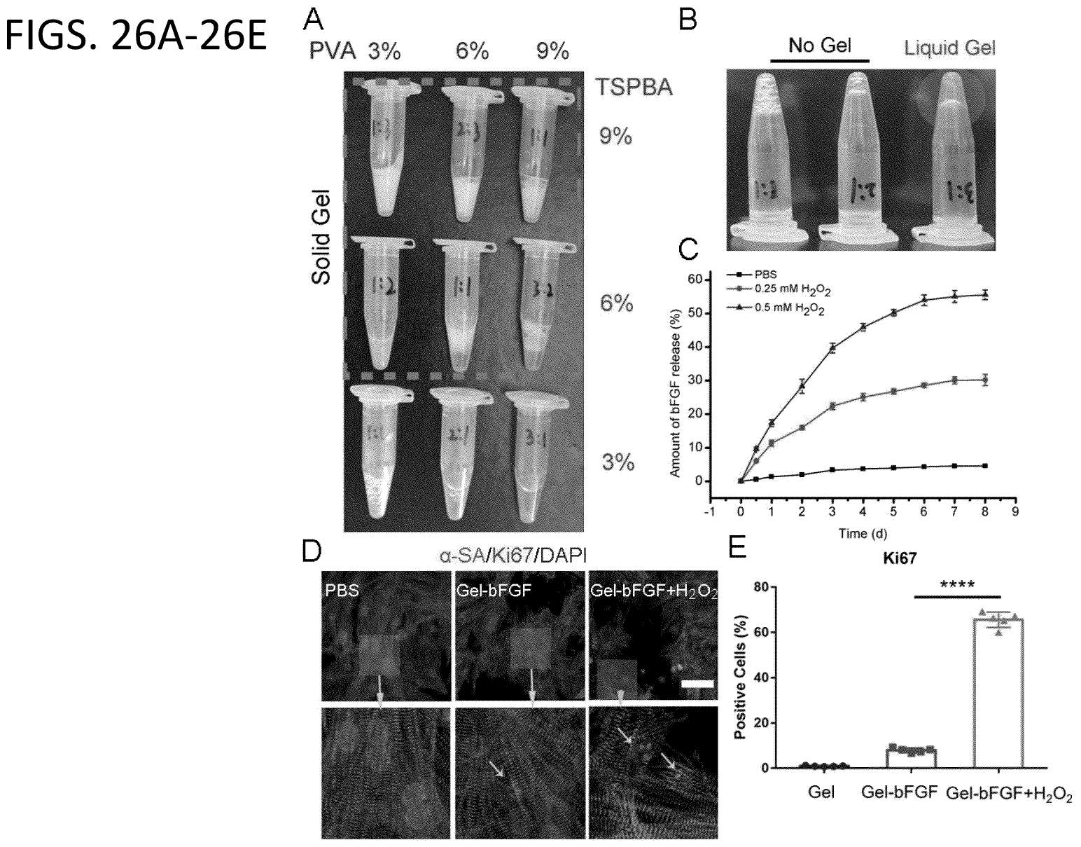

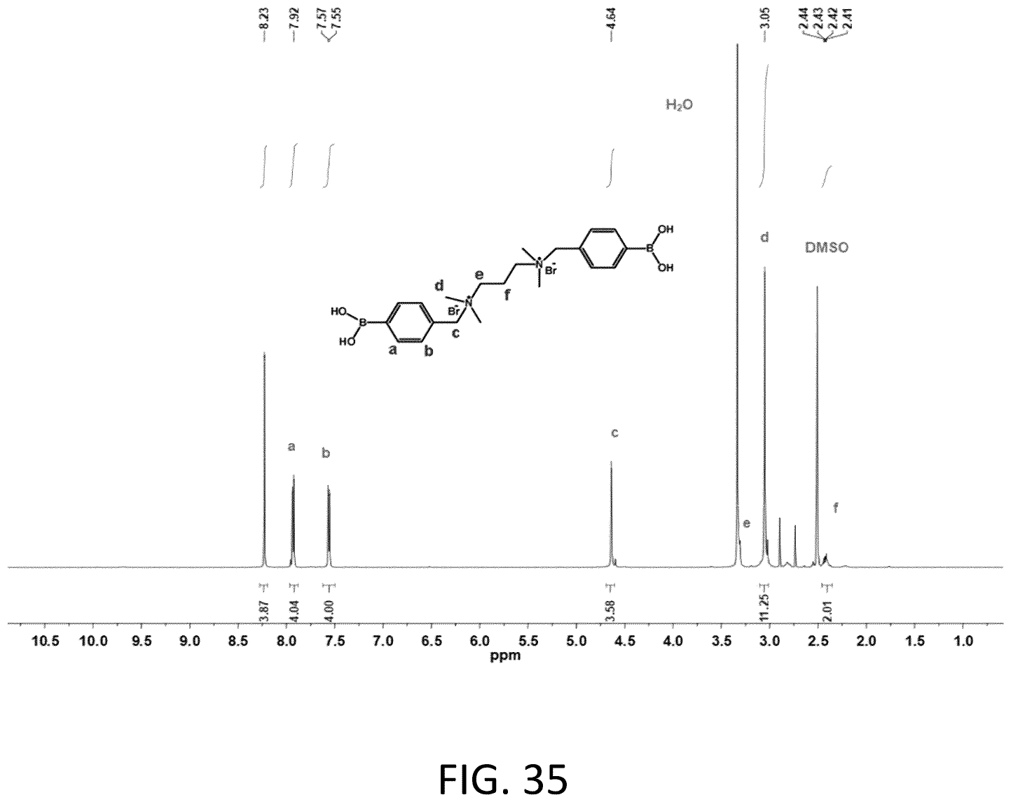

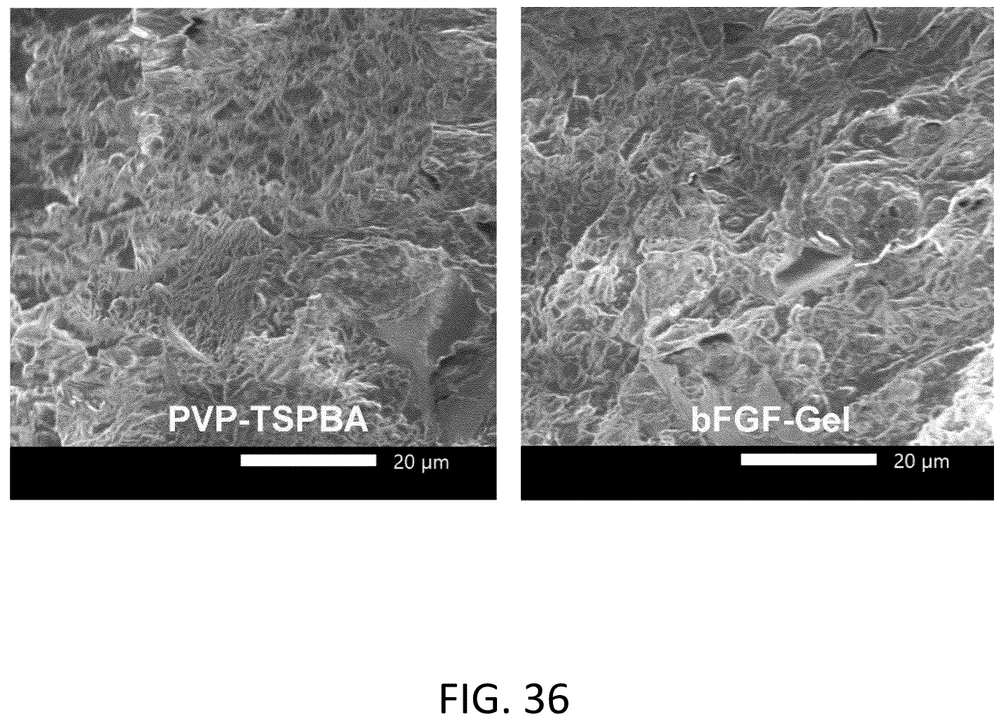

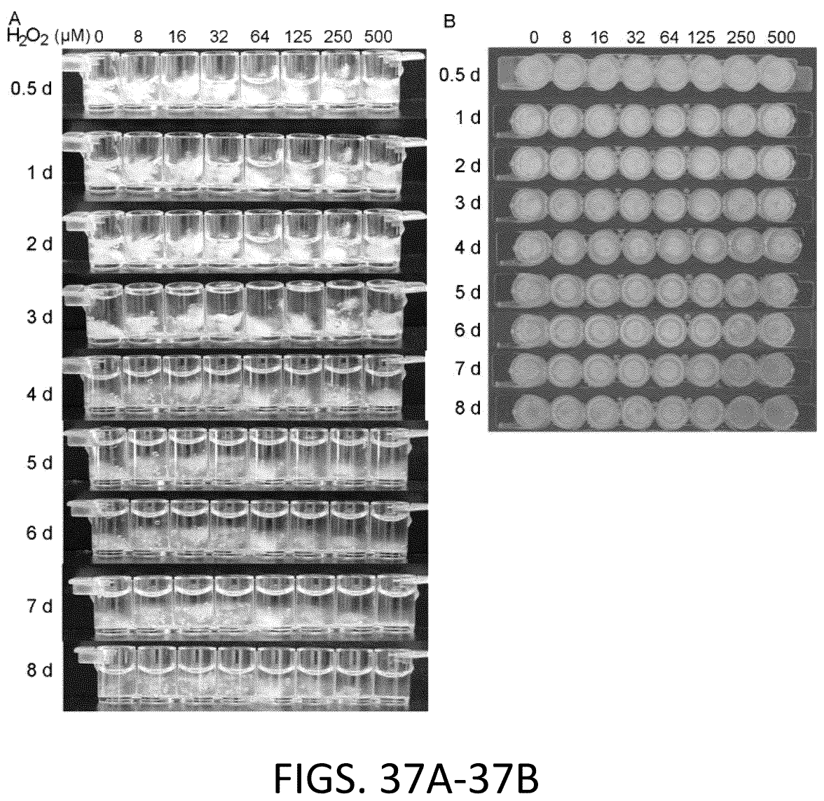

Fabrication of bFGF-loaded and ROS-responsive hydrogel. bFGF is unstable and will rapidly degrade right after delivery into the heart. To overcome this drawback, a ROS-responsive hydrogel was synthesized to deliver bFGF. PVA is one of the polyols that can react with benzoboric acid to form ROS-sensitive pinacol ester. PVA can further cross-link with N¹-(4-boronobenzyl)-N³-(4-boronophenyl)-N¹,N¹,N³,N³-tetramethylpropane-1,3- diaminium (TSPBA) to form a stable hydrogel. The ROS-responsive TSPBA linker was confirmed using ¹H-NMR (FIG. 35). TSPBA linker with quaternary ammonium groups were water-soluble, facilitating gel formation in an aqueous solution. As shown in FIG. 36, SEM images revealed the network structure of the hydrogel. To confirm ROS-triggered cleavage of PVA-TSPBA, the PVA-TSPBA gel was incubated with H₂O₂ at different concentrations. Concentration- and time-dependent PVA-TSPBA disassembly was evident (FIG. 37). After that, the effects of various concentrations of PVA and the TSPBA linker on the flexibility of gel were studied (FIG. 26A). When TSPBA was at a higher concentration (above 6%), a solid gel was formed regardless of PVA concentrations. In contrast, TSPBA at a concentration of 3% could cross-link with PVA (at 9%) to form a flexible gel. Continuous reduction in PVA concentrations caused no gel formation. To those ends, the optimal concentrations of PVA and TSPBA were selected as 9% and 3% (w/v), respectively (FIG. 26B).

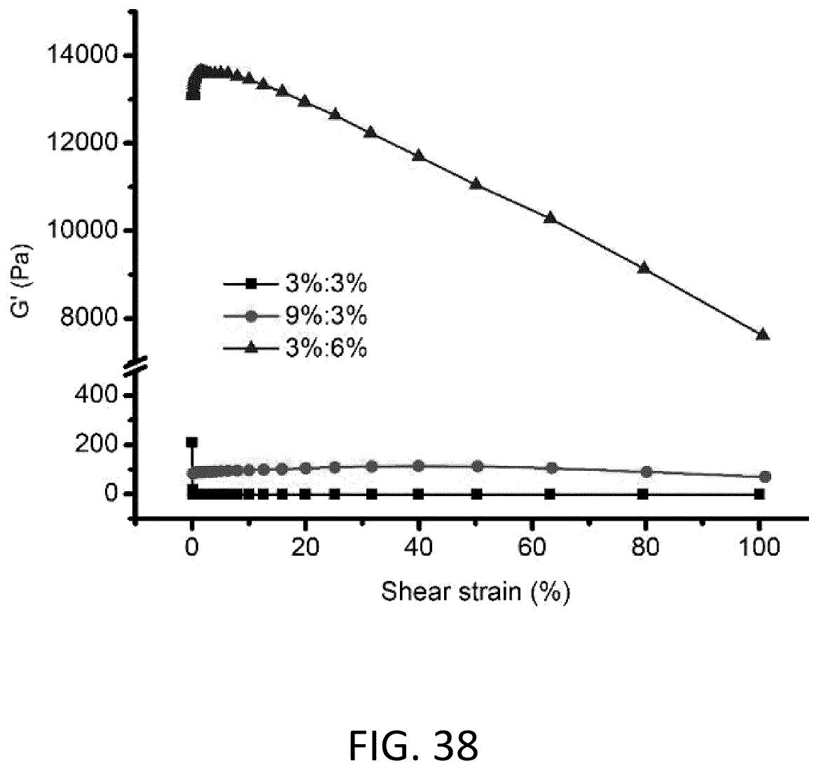

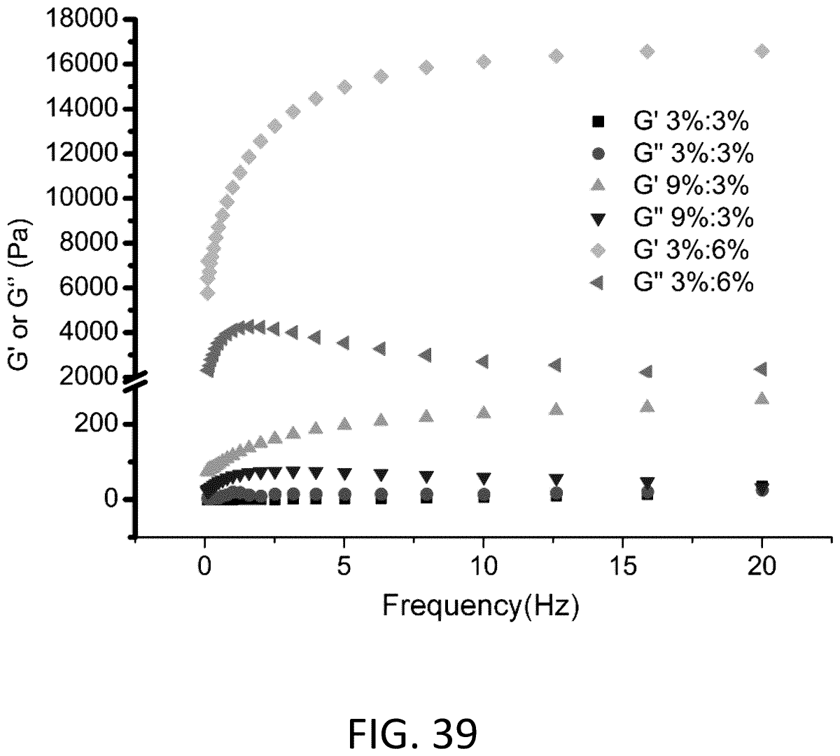

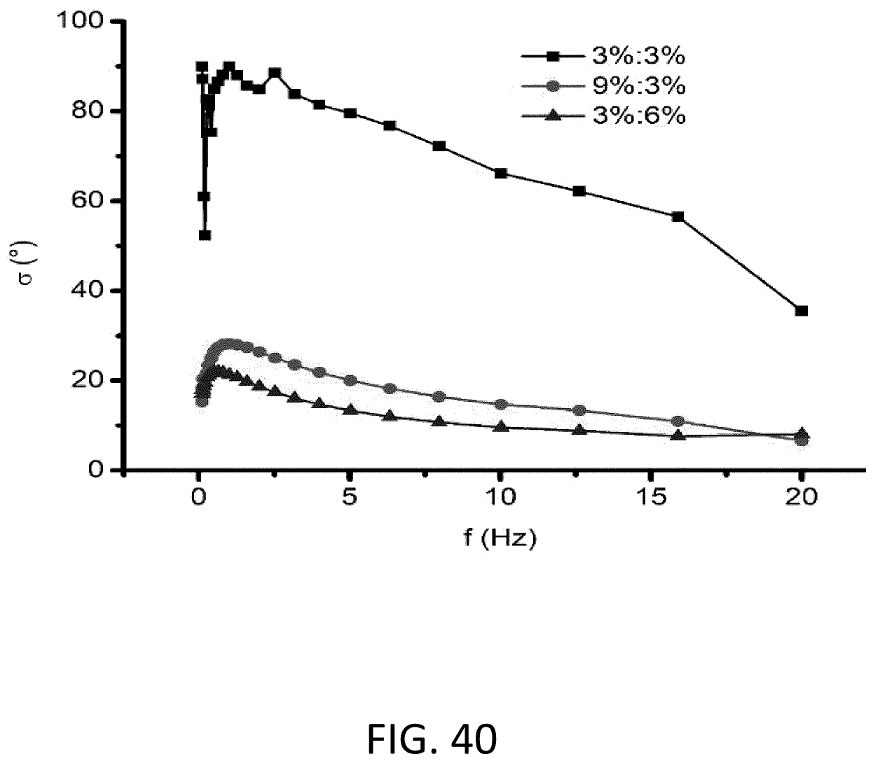

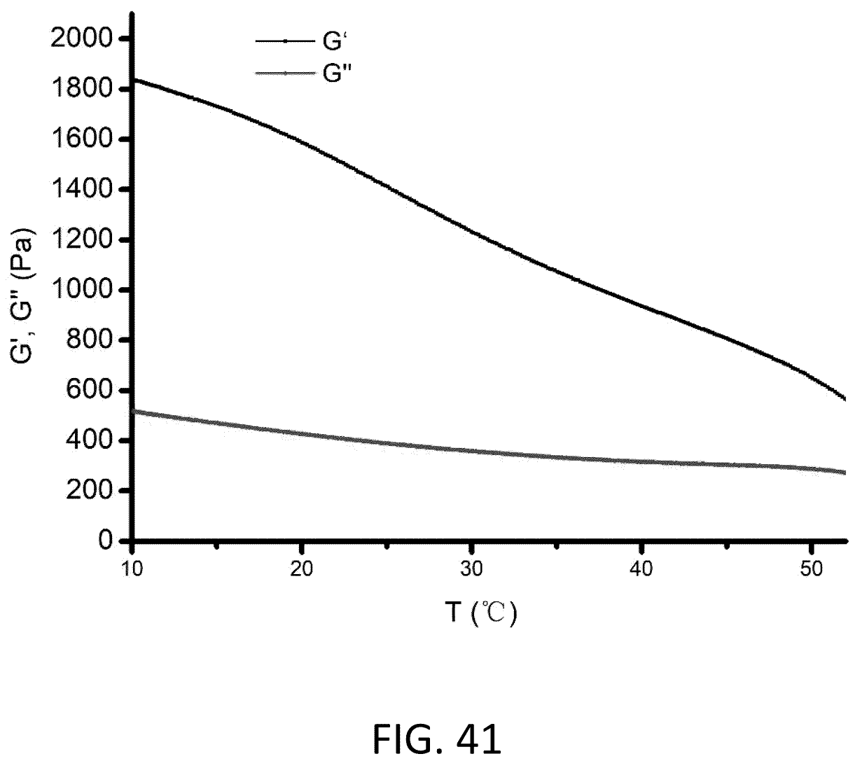

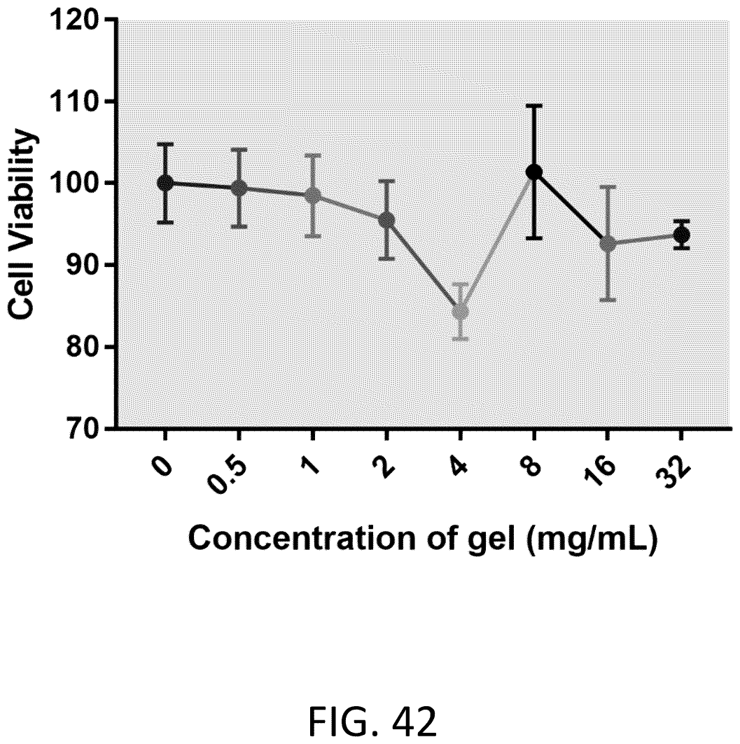

In addition, rheology studies were performed. Table 3 (below) and FIG. 38 summarize amplitude sweep results showing Gel 1 (PVP:TSPB=3%:3%) not having a measurable linear viscoelastic region (LVER) which was expected from its low viscosity and lack of rheological structure. Gel 2 (PVP:TSPBA=9%:3%) and 3 (PVP:TSPBA=3%:6%) did exhibit LVERs with elastic modulus (G′) decease with increasing % strain. Gel 2 has a lower G′ value (≈100 Pa; less solid-like properties) does have a very broad and large LVER (63.4% strain) suggesting appreciable elasticity (FIG. 39); whereas the much more stiff gel 3 (≈13,000 Pa; more solid-like properties) had a lower LVER (19.8% strain). Gel 1 showed G″>G′ up to 16 Hz to demonstrate its highly liquid nature. The G′G″-crossover at ≈17.5 Hz with frequency may be an artifact especially since both modulii values are very low. For Gel 2 and to a greater extent gel 3, clearly show G′>G″ across the frequency range (0.1-20 Hz) showing a dominant solid nature (viscoelastic solid). It may be helpful to note that the decreasing phase angle with frequency for both samples suggest the samples may become moderately more solid-like with higher impact or forceful (shorter timeframe) events (FIG. 40). Finally, the effect of temperature on the mechanical strength of the hydrogel was also studied. As shown in FIG. 41, it was found that the storage modulus of the sample decreases slowly as the temperature increases through the temperature sweep mode test. Then, bFGF releasing behavior of the hydrogel was evaluated. As shown in FIG. 26C, long-term release was observed at H₂O₂ concentrations of 0.25 mM and 0.5 mM. Increasing concentrations of H₂O₂ triggered more bFGF release from the gel. Additionally, the toxic effects of PVA-TSPBA on NRCMs were studied. Cell metabolic activity assay showed PVA-TSPBA did not affect the metabolic activity of NRCMs, indicating minimal cytotoxicity effects of PVA-TSPBA (FIG. 42). To mimic the oxidative stress after ischemia/reperfusion (I/R), 100 μM of H₂O₂ were added into the co-culture of NRCMs and Gel-bFGF. Compared to Gel-bFGF without H₂O₂, the introduction of H₂O₂ induced more NRCM proliferation owing to the release of bFGF from the gel (FIGS. 26D and E).

TABLE 3
Amplitude Sweep: Summary of elastic modulus change (G′)
at 25° C. to determine LVER (5% G′ loss*).

| | | | | | % strain to be |
|---|---|---|---|---|---|
| | | | | | used as input |
| | | G′ (calculated 5% | LVERupper | LVERupper | for frequency |
| Samples | G′ (plateau | decrease G′_{(plateau or max)}) | limit* | limit | sweep |
| PVP:TSPBA | or max) Pa | Pa | (actual (Pa)) | % strain | (Test 3) |
| 3%:3% | Not determined | Not determined | Not determined | Not determined | 0.1 |
| 9%:3% | 112.2 | 106.6 | 105.2 | 63.4 | 5 |
| 3%:6% | 13,640 | 12,958 | 12,940 | 19.8 | 5 |

*LVER is defined as % strain to give 5% loss G′.

### Example 16

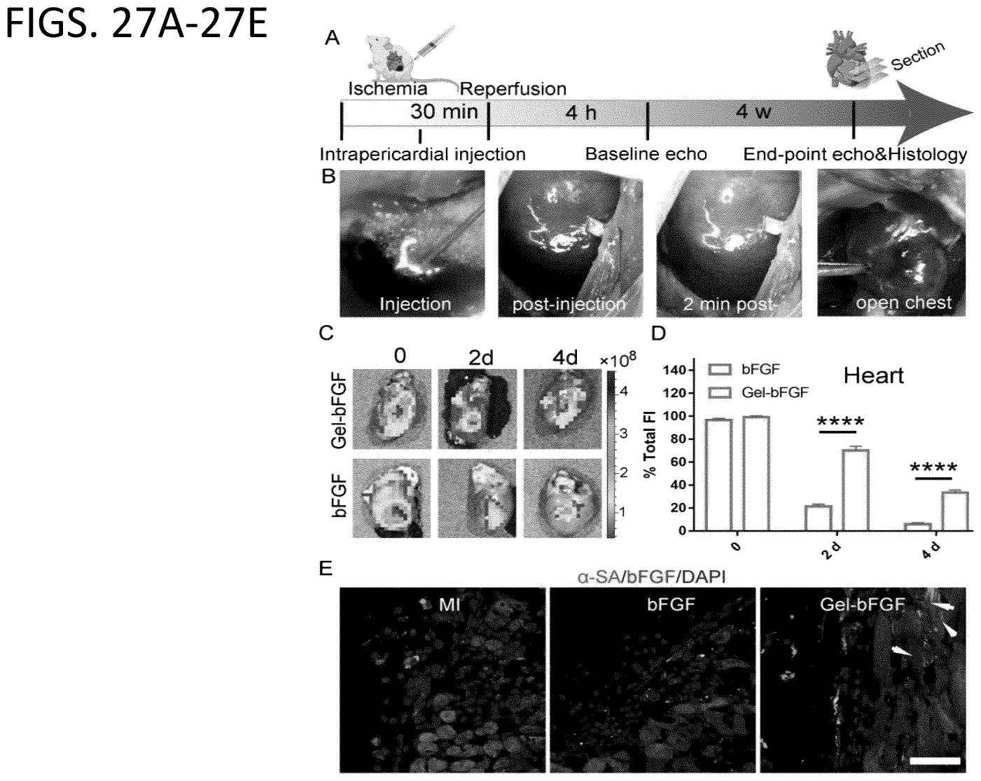

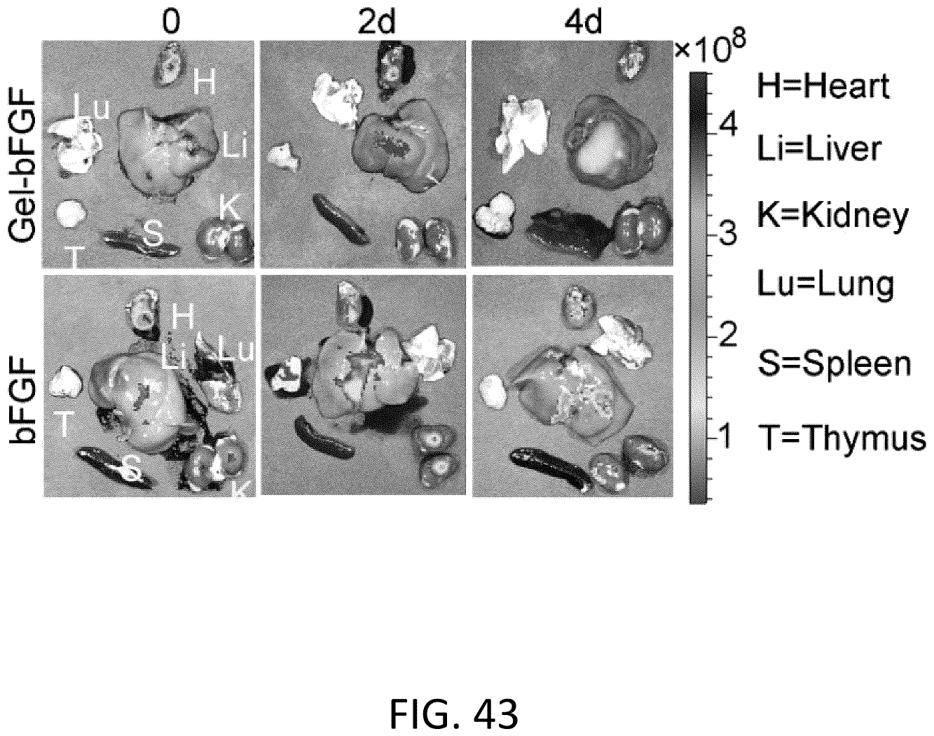

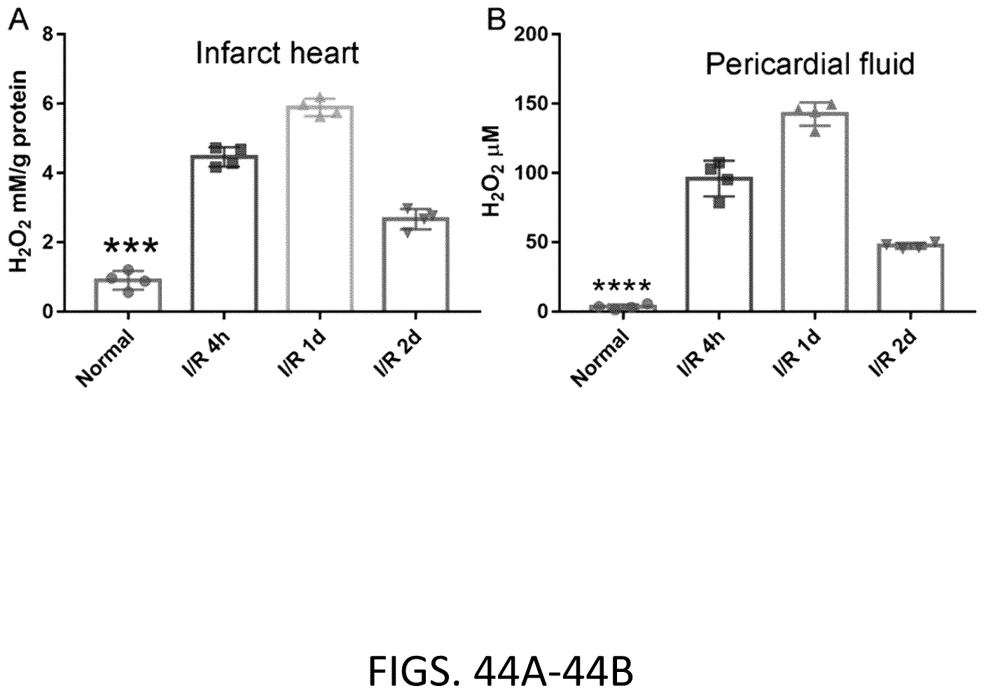

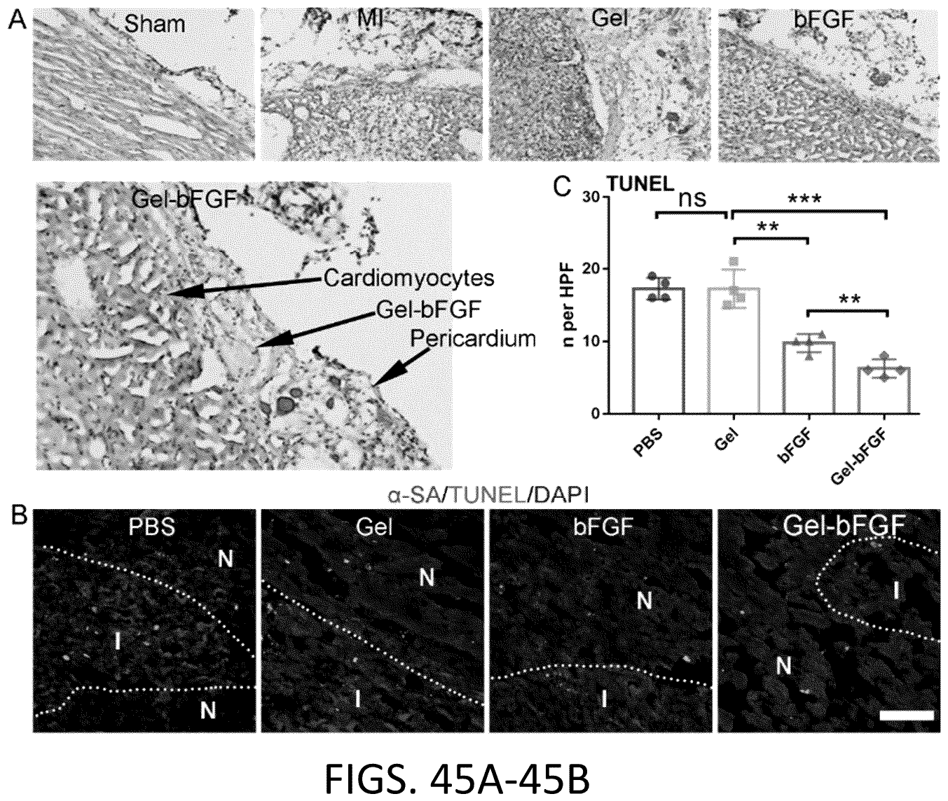

iPC injection of ROS-responsive hydrogel and bFGF biodistribution. After in vitro characterization, animal studies were conducted (FIG. 27A). All animal studies were approved by the Institutional Animal Usage and Care Committee of North Carolina State University. The feasibility of iPC injection of hydrogel was first confirmed. Alcian Blue were loaded into the PVA-TSPBA gel for visualization during injection (FIG. 27B; video can be made available upon request). iPC injection of hydrogel can be performed and the blue dye spread to the whole apex of heart within minutes. The biodistribution of Gel-bFGF after iPC administration was tested in I/R rats. Compared with bFGF delivered in saline, the ROS-responsive hydrogel enhanced the retention of bFGF in the heart (FIGS. 27C and D; FIG. 43). Furthermore, the fate of injected Gel-bFGF was studied by immunostaining of sectioned I/R hearts. Increased ROS levels after I/R were first verified. The concentrations of ROS were the highest 1 day after I/R injury (about 6 mM per g protein in the myocardium and 150 μM per mL in pericardial fluid) and decreased afterwards (FIG. 44). It was evident that released bFGF penetrated epicardium while a portion of bFGF remained in the pericardial cavity (FIG. 27E). In addition, H&E stain confirmed the presence of gel in the pericardial cavity (FIG. 45A). Such data demonstrated that iPC injection in ROS-responsive hydrogel was an efficient way to deliver bFGF to the injured heart and the biodistribution of FGF favored cardiac repair activities.

### Example 17

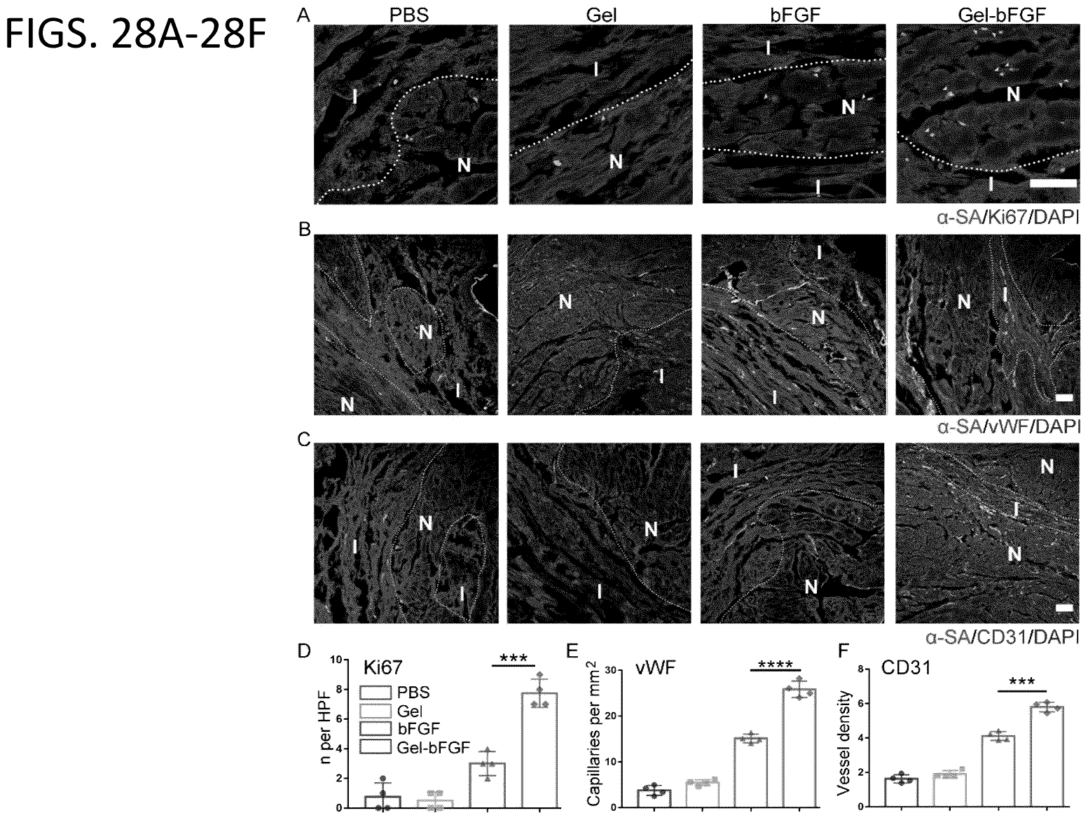

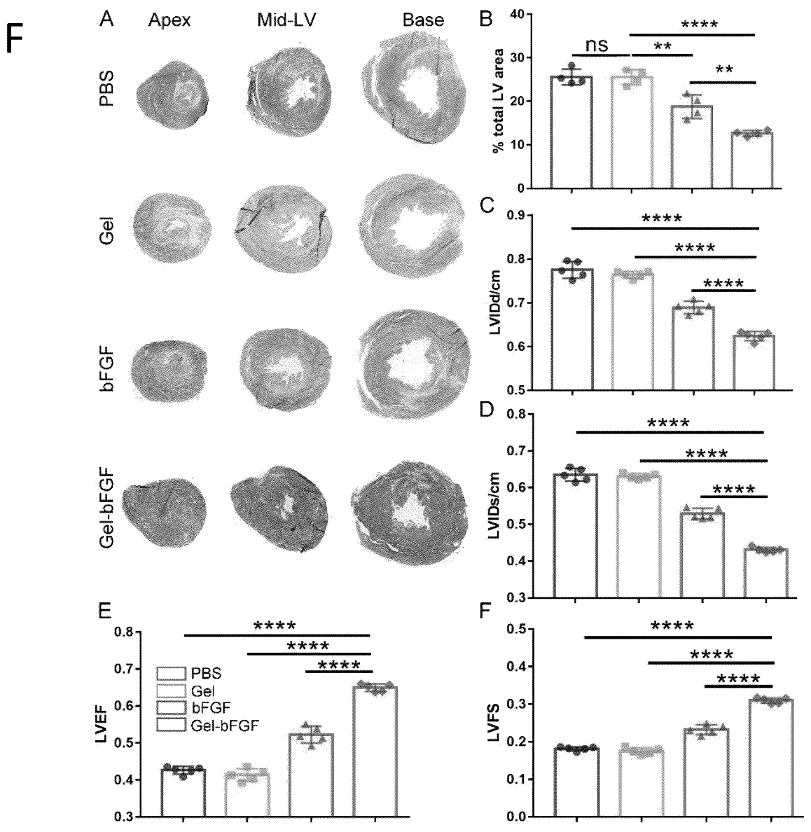

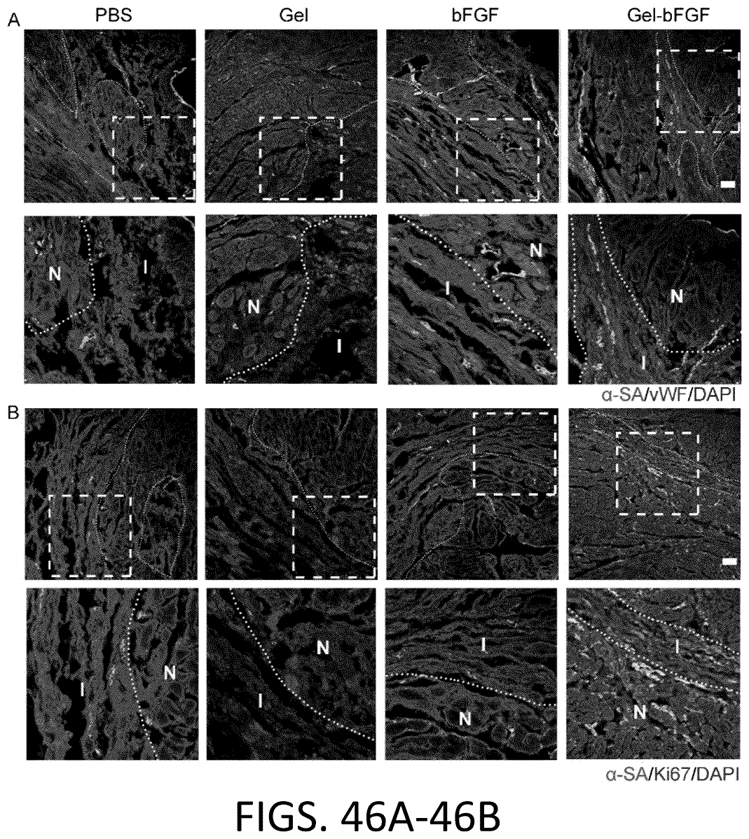

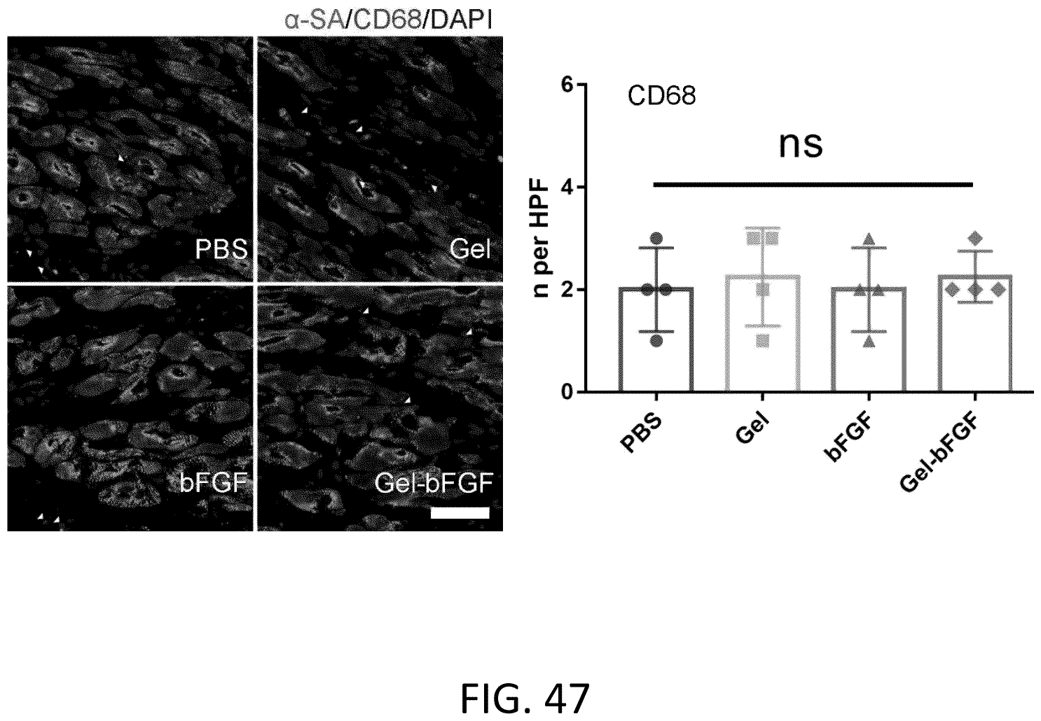

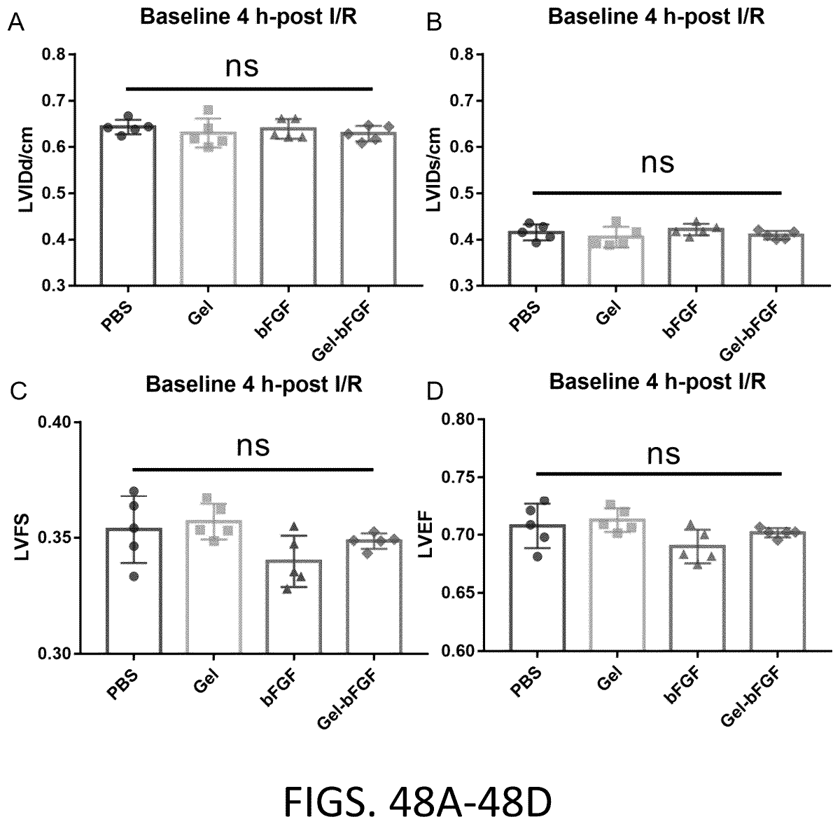

Therapeutic efficacy of iPC injection of Gel-bFGF in a rat model of I/R injury. iPC injection of Gel-bFGF reduced the apoptosis (indicated by TUNEL positivity) of cardiomyocytes (labeled by sarcomeric actin (α-SA)) (FIGS. 45B and C). Injection of Gel-bFGF promoted endogenous cell proliferation. The number of Ki67-positive cells was higher in the peri-infarct region with the treatment of Gel-bFGF (FIGS. 28A and D). Furthermore, Gel-bFGF increased the numbers of vWF-positive vasculatures (FIGS. 28B and E; FIG. 46A). The results of CD31 staining were consistent with vWF staining (FIGS. 28C and F; FIG. 46B). In addition, injection of Gel-bFGF did not exacerbate inflammation in the post-MI heart as the numbers of CD68-positive macrophages were indistinguishable among the groups (FIG. 47). Heart morphometry on Masson's trichrome-stained heart sections revealed the protective effects of Gel-bFGF treatment, which resulted in a small scar size but more viable myocardium (FIGS. 29A and B). Echocardiography was employed to evaluate cardiac functions after various treatments. The initial injury was the same at baseline for all groups (FIG. 48). An improvement in cardiac morphology was found with Gel-bFGF treatment, as indicated by a reduction in LV hypertrophy (FIGS. 29C and D). In line with the morphological benefits, iPC injection of Gel-bFGF augmented left ventricular ejection fraction (LVEF) and fractional shortening (FS) (FIGS. 29E and F).

### Example 18

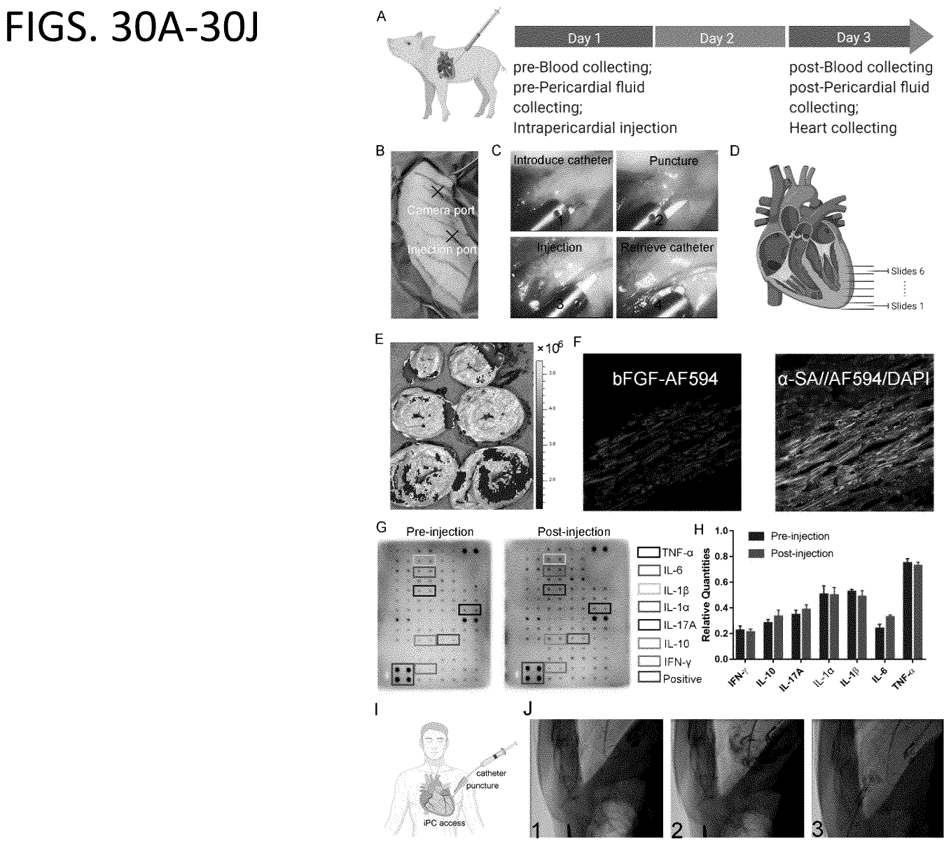

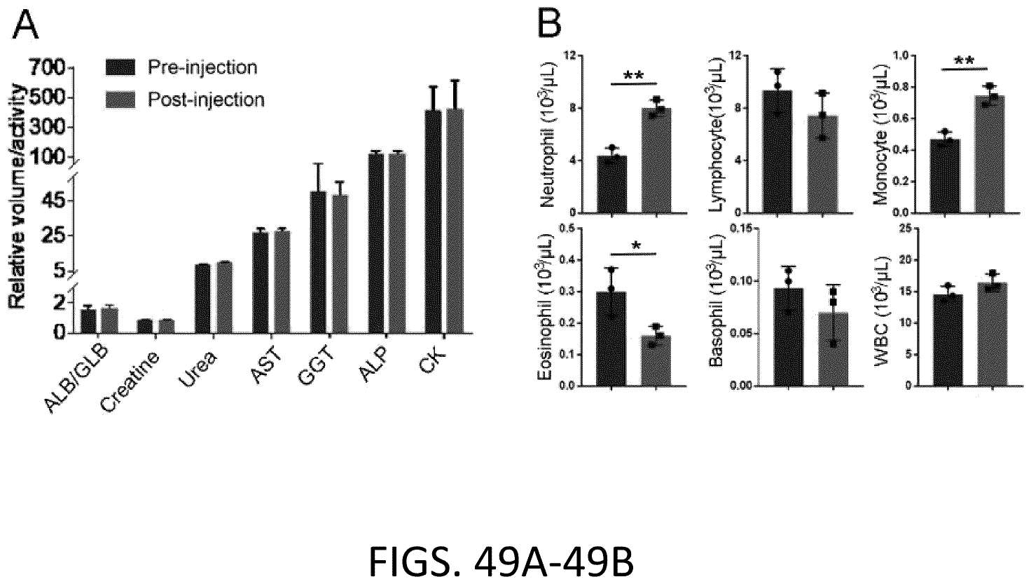

Feasibility, safety, and biodistribution of minimally invasive iPC Gel-bFGF injection in pigs. Next, minimally invasive injections of Gel-bFGF into the pericardial cavity of pigs were performed (FIG. 30A). Two small incisions were first made on the left chest as the ports of trocar, which were used for the introduction of thoracoscope and custom-made delivery tube, subsequently (FIGS. 30B and C; video can be made available upon request). Three days after treatment heart were collected and sliced (FIG. 30D). IVIS imaging revealed that a large portion of bFGF was still retained in the pericardial space while the remaining could be found in the myocardium (FIG. 30E). Histology confirmed the presence of bFGF in the myocardium (FIG. 30F). iPC injection of Gel-bFGF had minimal adverse effects on liver function (AST, Crnetine, ALB/GLB, and GGT), kidney (BUN) function, or heart (CK) function (FIG. 49A). Hemotology analysis suggested some inflammatory reaction (FIG. 49B) which could be due to the procedure itself. The change in inflammatory cytokines (including IFN-γ, IL-1α, IL-1β, IL-17A, IL-10, IL-6, and TNF-α) in the pericardial fluid (FIGS. 30G and H) were further studied. No changes were detected.

### Example 19

Minimally invasive iPC access in a human patient. Additionally, the feasibility of minimally invasive iPC access in a human patient was demonstrated (FIGS. 30I and J; videos can be made available upon request) who underwent the standard LARIAT procedure. The procedure can be performed with one small incision under fluoroscope, which is available in most of the hospitals.

As referenced above, videos capturing various aspects of the embodiments of the present disclosure can be made available upon request. These videos pertain to the following:

iPC injection in a mouse model of myocardial infarction (MI). After induction of MI model by LAD ligation, iPC injection was performed with a 0.3 mL syringe with a low angle puncture into the pericardium. The injection volume is 20 μL.

iPC injection in a rat model of myocardial infarction (MI). After induction of MI model by LAD ligation, iPC injection was performed with a 0.3 mL syringe with a low angle puncture into the pericardium. A blue dye was employed to confirm the contents were injected into the pericardial cavity but not into the myocardium. The injection volume was 50 μL.

Minimally invasive iPC injection in porcine with 2 incisions. To access to the pericardial cavity of pigs, two trocars were placed at the 3^{rd} and the 7^{th} intercostal for camera and injection catheter entry respectively. iPC injection was performed with a puncture into the pericardial cavity with a 16G catheter. The injection volume is 5 mL in pigs.

Minimally invasive iPC access in human patients with only one incision. First, a lateral view angiogram is obtained which reveals the location of the apex of the right ventricle (video can be made available upon request). Next, using a small bore (0.018″) access needle, iodinated contrast is used to mark the border of the pericardial space. After entering the space with a needle, a wire was advanced into the pericardial space (video can be made available upon request). Next, a serial dilations were performed prior to the introduction of the access sheath that can be used for intrapericardial injection.

Representative confocal Z-stack images showing in vivo differentiation of IPS-CPCs into cardiomyocytes 4 weeks after iPC injection. iPS-CPCs were tagged with GFP, and cardiomyocytes were marked with α-sarcomeric actinin (α-SA) in red.

Representative confocal Z-stack images confirming epicardial uptake of exosomes after iPC injection. Exosomes were pre-labeled with DiD (red), and epicardium was labeled with podoplanin (green).

1. A method for treating or preventing a cardiac injury in a subject, the method comprising:
delivering a hydrogel-based composition into a portion of a pericardial cavity of a subject, wherein the composition comprises at least one therapeutic agent; and
improving at least one aspect of myocardial cells or tissue in the subject.

2. The method of claim 1, wherein the method is preformed using an imaging device, and wherein the composition is biocompatible.

3. (canceled)

4. The method of claim 1, wherein the composition is delivered via intrapericardial (iPC) injection.

5. (canceled)

6. The method of claim 1, wherein the method is performed after the subject has suffered a myocardial infarction.

7-8. (canceled)

9. The method of claim 1, wherein delivery of the composition to the pericardial cavity of the subject causes the hydrogel-based composition to degrade and release the at least one therapeutic agent.

10. The method of claim 1, wherein the at least one therapeutic agent comprises a growth factor, a microRNA, a microRNA mimic, an exosome, a cell, and any combinations or derivatives thereof.

11. The method of claim 10, wherein:
(i) the growth factor is Fibroblast Growth Factor (FGF);
(ii) the microRNA mimic is miR-21, miR-125, miR-146, or any combination thereof;
(iii) the exosome is a mesenchymal stem cell (MSC)-derived exosome;
(iv) the cell is an induced pluripotent stem cell-derived cardiac progenitor cell (iPS-CPCs); or
(v) wherein the cell is a mesenchymal stem cell (MSC).

12. The method of claim 1, wherein the hydrogel-based composition is at least one of a hyaluronic acid (HA)-based hydrogel, a decellularized extracellular matrix (ECM) hydrogel, a polyvinyl alcohol (PVA)-based hydrogel, and any combinations or derivatives thereof.

13. The method of claim 1, wherein the at least one aspect of myocardial cells or tissue that is improved comprises increased myocardiocyte survival, decreased myocardiocyte apoptosis, increased myocardiocyte proliferation, increased myocardial differentiation, increased angiogenesis, reduced ischemia, improved myocardiocyte function, and any combinations thereof.

14. (canceled)

15. A hydrogel-based composition comprising for treating a cardiac injury, the composition comprising:
a hydrogel component; and
at least one therapeutic agent.

16. The composition of claim 15, wherein the hydrogel component comprises at least one of a hyaluronic acid (HA)-based hydrogel component, a decellularized extracellular matrix (ECM) hydrogel component, a polyvinyl alcohol (PVA)-based hydrogel component, and any combinations or derivatives thereof.

17. The composition of claim 15, wherein the at least one therapeutic agent comprises a growth factor, a microRNA, a microRNA mimic, an exosome, a stem cell, and any combinations or derivatives thereof.

18. The composition of claim 15, wherein the at least one therapeutic agent comprises Fibroblast Growth Factor (FGF), and wherein the hydrogel component comprises a polyvinyl alcohol (PVA)-based hydrogel component.

19. The composition of claim 18, wherein the hydrogel-based composition further comprises N1-(4-boronobenzyl)-N3-(4-boronophenyl)-N1,N1,N3,N3-tetramethylpropane-1,3-diaminium (TSPBA), and wherein exposure of the composition to reactive oxygen species (ROS) cleaves the TSPBA from the PVA-based hydrogel component and releases the at least one therapeutic agent.

20. (canceled)

21. The composition of claim 15, wherein the at least one therapeutic agent comprises miR-21, miR-125, miR-146, or any combination thereof, and wherein the hydrogel component comprises a decellularized extracellular matrix (ECM) hydrogel component.

22. (canceled)

23. The composition of claim 21, wherein the miR-21, miR-125, miR-146, or any combination thereof, is chemically modified with an HIV TAT peptide.

24. The composition of claim 21, wherein the ECM hydrogel component is present in the composition at a concentration ranging from about 5 mg/ml to about 25 mg/ml.

25. The composition of claim 15, wherein the at least one therapeutic agent comprises a mesenchymal stem cell (MSC)-derived exosome, and wherein the hydrogel component comprises a hyaluronic acid (HA)-based hydrogel component.

26. The composition of claim 25, wherein the HA-based hydrogel component comprises methacrylic anhydride (MA) cross-linked to HA.

27. The composition of claim 15, wherein the at least one therapeutic agent comprises a mesenchymal stem cell (MSC), and wherein the hydrogel component comprises a decellularized extracellular matrix (ECM) hydrogel component.

28-31. (canceled)
